SEQUENCE LISTING (24 sequences)

SEQ ID NO: 1 - DNA, 3489 bp, Infectious Bronchitis Virus
atgctggtgaccccactgctgctggtgacactgctgtgcgcactgtgctccgccgctctgtacgatagctccagctacgtgtactactaccagagcgcattccggccccctgatggatggcacctgcacggcggagcctacgctgtggtgaacatctccagcgagagcaacaatgctggctccagctccggatgcacagtggggatcattcacgggggcagagtggtgaatgcaagctccattgcaatgactgcccccagctccggaatggcatggagctccagccagttttgcaccgcctactgcaactttagcgataccacagtgttcgtgacacactgctacaagcacggagggtgcccaatcactggaatgctgcagcagcacagcattagggtgtccgcaatgaaaaacgggcagctgttctacaatctgacagtgagcgtggccaagtaccccacttttaaatccttccagtgcgtgaacaatctgaccagcgtgtacctgaacggcgatctggtgtacaccagcaatgctactaccgacgtgacatccgcaggagtgtactttaaggccggcggacctatcacatacaaagtgatgcgggaagtgagagcactggcctacttcgtgaatggcactgctcaggatgtgattctgtgcgatggctccccaaggggactgctggcatgccagtacaacaccggcaatttcagcgacggattttaccccttcacaaactccagcctggtgaagcagaaatttatcgtgtaccgcgagaacagcgtgaatacaactttcacactgcacaacttcacttttcacaatgaaaccggagccaaccccaatcctagcggggtgcagaacattcagacataccagacccagacagctcagagcgggtactacaacttcaatttttccttcctgtccagctttgtgtacaaggagagcaacttcatgtacggcagctaccacccatcctgcaattttcggctggaaacaatcaacaatgggctgtggttcaactccctgagcgtgtccattgcttacggccctctgcagggcggctgcaaacagagcgtgttttccggaagagccacctgctgctacgcttactcctacggagggccactgctgtgcaagggggtgtacagcggcgagctggatcacaatttcgaatgcggactgctggtgtacgtgaccaaaagcggcggcagcagaatccagactgccaccgagccacccgtgatcacacagcacaactacaacaatattacactgaacacttgcgtggactacaatatctacgggagaactggccagggattcattaccaacgtgacagatagcgctgtgtcctacaattacctggctgacgcaggcctggcaatcctggataccagcggcagcatcgacatttttgtggtgcagtccgagtacggcctgaactactacaaagtgaatccctgcgaagatgtgaaccagcagttcgttgtgagcgggggcaaactggtgggaatcctgacaagccggaatgagactgggtcccagctgctggaaaaccagttctacatcaagatcactaacggaaccagaaggttccgccggagcatcacagagtccgtggaaaactgcccttacgtgtcctacgggaagttttgcattaaaccagacggcagcatcgccactattgtgcccaagcagctggagcagtttgtggctcctctgctgaacgtgaccgaaaatgtgctgatcccaaacagcttcaatctgacagtgactgatgagtacattcagaccaggatggacaaagtgcagatcaactgcctgcagtacatttgcgggaacagcctggaatgccgcaatctgttccagcagtacggccctgtgtgcgataacatcctgagcgtggtgaacagcgtgggccagaaggaggacatggaactgctgaacttttactccagcactaaacccgccggcttcaacacccctgtgctgagcaatgtgtccaccggagagtttaatatctccctgttcctgaccacaccatccagccccagaaggcgcagctttattgaggatctgctgttcacaagcgtggaatccgtggggctgcccactgatgacgcttacaagaactgcaccgcaggccctctgggattcctgaaagacctggcctgcgctcgcgagtacaatggcctgctggtgctgcctccaatcattacagctgaaatgcagatcctgtacacttccagcctggtggctagcatggcatttggagggatcactgcagccggggcaattcccttcgccacccagctgcaggcaaggatcaaccacctgggcattacacagtccctgctgctgaagaaccaggagaaaatcgctgccagcttcaataaggctattgggcacatgcaggaaggcttccgcagcacttccctggcactgcagcagatccaggatgtggtgaacaagcagtccgccattctgaccgagacaatggctagcctgaacaaaaattttggcgccatctccagcgtgatccaggaaatttaccagcagctggatgctatccaggcaaacgcccaggtggacaggctgattacaggacgcctgtccagcctgagcgtgctggcttccgcaaagcaggcagagtacatccgggtgtcccagcagagagagctggccacacagaagatcaacgaatgcgtgaaaagccagtccattcggtacagcttctgcgggaatggcagacacgtgctgactatccctcagaacgccccaaatggcatcgtgtttattcacttcagctacacccccgactcctttgtgaacgtgacagctatcgtgggattctgcgtgaagccagccaatgcttcccagtacgctattgtgcctgcaaacggaagagggatctttattcaagtgaatggaagctactacatcactgcaagggatatgtacatgcctcgcgccatcaccgctggggacattgtgactctgaccagctgccaggccaactacgtgtccgtgaataaaaccgtgatcactaccttcgtggataacgatgactttgatttcaatgacgagctgagcaagtggtggaacgacacaaaacacgaactgcctgattttgacaagttcaattacactgtgccaatcctggatattgacagcgagatcgataggattcagggagtgatccaggggctgaacgatagcctgattgacctggaaaaactgtccatcctgaagacatacattaaatggccctggtacgtgtggctggcaatcgcctttgctaccatcattttcatcctgattctgggatgggtgttctttatgacagggtgctgcggctgctgctgcggatgctttgggattatgcccctgatgagcaagtgcgggaagaaatccagctactacacaactttcgataacgacgtggtgaccgagcagtaccgccctaagaaaagcgtgtga

SEQ ID NO: 2 - protein, 1162 aa, Infectious Bronchitis Virus
MLVTPLLLVTLLCALCSAALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNATTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTRRFRRSITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLACAREYNGLLVLPPIITAEMQILYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 3 - DNA, 3423 bp, Artificial Sequence
  chimeric sequence of modified infectious bronchitis virus and vesicular stomatitis virus
atgctggtgaccccactgctgctggtgacactgctgtgcgcactgtgctccgccgctctgtacgatagctccagctacgtgtactactaccagagcgcattccggccccctgatggatggcacctgcacggcggagcctacgctgtggtgaacatctccagcgagagcaacaatgctggctccagctccggatgcacagtggggatcattcacgggggcagagtggtgaatgcaagctccattgcaatgactgcccccagctccggaatggcatggagctccagccagttttgcaccgcctactgcaactttagcgataccacagtgttcgtgacacactgctacaagcacggagggtgcccaatcactggaatgctgcagcagcacagcattagggtgtccgcaatgaaaaacgggcagctgttctacaatctgacagtgagcgtggccaagtaccccacttttaaatccttccagtgcgtgaacaatctgaccagcgtgtacctgaacggcgatctggtgtacaccagcaatgctactaccgacgtgacatccgcaggagtgtactttaaggccggcggacctatcacatacaaagtgatgcgggaagtgagagcactggcctacttcgtgaatggcactgctcaggatgtgattctgtgcgatggctccccaaggggactgctggcatgccagtacaacaccggcaatttcagcgacggattttaccccttcacaaactccagcctggtgaagcagaaatttatcgtgtaccgcgagaacagcgtgaatacaactttcacactgcacaacttcacttttcacaatgaaaccggagccaaccccaatcctagcggggtgcagaacattcagacataccagacccagacagctcagagcgggtactacaacttcaatttttccttcctgtccagctttgtgtacaaggagagcaacttcatgtacggcagctaccacccatcctgcaattttcggctggaaacaatcaacaatgggctgtggttcaactccctgagcgtgtccattgcttacggccctctgcagggcggctgcaaacagagcgtgttttccggaagagccacctgctgctacgcttactcctacggagggccactgctgtgcaagggggtgtacagcggcgagctggatcacaatttcgaatgcggactgctggtgtacgtgaccaaaagcggcggcagcagaatccagactgccaccgagccacccgtgatcacacagcacaactacaacaatattacactgaacacttgcgtggactacaatatctacgggagaactggccagggattcattaccaacgtgacagatagcgctgtgtcctacaattacctggctgacgcaggcctggcaatcctggataccagcggcagcatcgacatttttgtggtgcagtccgagtacggcctgaactactacaaagtgaatccctgcgaagatgtgaaccagcagttcgttgtgagcgggggcaaactggtgggaatcctgacaagccggaatgagactgggtcccagctgctggaaaaccagttctacatcaagatcactaacggaaccgccgccttcgccgccagcatcacagagtccgtggaaaactgcccttacgtgtcctacgggaagttttgcattaaaccagacggcagcatcgccactattgtgcccaagcagctggagcagtttgtggctcctctgctgaacgtgaccgaaaatgtgctgatcccaaacagcttcaatctgacagtgactgatgagtacattcagaccaggatggacaaagtgcagatcaactgcctgcagtacatttgcgggaacagcctggaatgccgcaatctgttccagcagtacggccctgtgtgcgataacatcctgagcgtggtgaacagcgtgggccagaaggaggacatggaactgctgaacttttactccagcactaaacccgccggcttcaacacccctgtgctgagcaatgtgtccaccggagagtttaatatctccctgttcctgaccacaccatccagccccagaaggcgcagctttattgaggatctgctgttcacaagcgtggaatccgtggggctgcccactgatgacgcttacaagaactgcaccgcaggccctctgggattcctgaaagacctggcctgcgctcgcgagtacaatggcctgctggtgctgcctccaatcattacagctgaaatgcagatcctgtacacttccagcctggtggctagcatggcatttggagggatcactgcagccggggcaattcccttcgccacccagctgcaggcaaggatcaaccacctgggcattacacagtccctgctgctgaagaaccaggagaaaatcgctgccagcttcaataaggctattgggcacatgcaggaaggcttccgcagcacttccctggcactgcagcagatccaggatgtggtgaacaagcagtccgccattctgaccgagacaatggctagcctgaacaaaaattttggcgccatctccagcgtgatccaggaaatttaccagcagctggatgctatccaggcaaacgcccaggtggacaggctgattacaggacgcctgtccagcctgagcgtgctggcttccgcaaagcaggcagagtacatccgggtgtcccagcagagagagctggccacacagaagatcaacgaatgcgtgaaaagccagtccattcggtacagcttctgcgggaatggcagacacgtgctgactatccctcagaacgccccaaatggcatcgtgtttattcacttcagctacacccccgactcctttgtgaacgtgacagctatcgtgggattctgcgtgaagccagccaatgcttcccagtacgctattgtgcctgcaaacggaagagggatctttattcaagtgaatggaagctactacatcactgcaagggatatgtacatgcctcgcgccatcaccgctggggacattgtgactctgaccagctgccaggccaactacgtgtccgtgaataaaaccgtgatcactaccttcgtggataacgatgactttgatttcaatgacgagctgagcaagtggtggaacgacacaaaacacgaactgcctgattttgacaagttcaattacactgtgccaatcctggatattgacagcgagatcgataggattcagggagtgatccaggggctgaacgatagcctgattgacctggaaaaactgtccatcctgaagacatacattaaatcctccatcgcttccttcttcttcatcatcggcctgatcatcggactgtttctggtgctgagggtgggcatctacctgtgcatcaagctgaagcacactaagaagaggcagatctacaccgacatcgagatgaacaggctgggcaagtga

SEQ ID NO: 4 - protein, 1140 aa, Artificial Sequence
  chimeric sequence of modified infectious bronchitis virus and vesicular stomatitis virus
MLVTPLLLVTLLCALCSAALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNATTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTAAFAASITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLACAREYNGLLVLPPIITAEMQILYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKSSIASFFFIIGLIIGLFLVLRVGIYLCIKLKHTKKRQIYTDIEMNRLGK

SEQ ID NO: 5 - DNA, 3423 bp, Artificial Sequence
  chimeric sequence of modified infectious bronchitis virus and vesicular stomatitis virus
atgctggtgaccccactgctgctggtgacactgctgtgcgcactgtgctccgccgctctgtacgatagctccagctacgtgtactactaccagagcgcattccggccccctgatggatggcacctgcacggcggagcctacgctgtggtgaacatctccagcgagagcaacaatgctggctccagctccggatgcacagtggggatcattcacgggggcagagtggtgaatgcaagctccattgcaatgactgcccccagctccggaatggcatggagctccagccagttttgcaccgcctactgcaactttagcgataccacagtgttcgtgacacactgctacaagcacggagggtgcccaatcactggaatgctgcagcagcacagcattagggtgtccgcaatgaaaaacgggcagctgttctacaatctgacagtgagcgtggccaagtaccccacttttaaatccttccagtgcgtgaacaatctgaccagcgtgtacctgaacggcgatctggtgtacaccagcaatgctactaccgacgtgacatccgcaggagtgtactttaaggccggcggacctatcacatacaaagtgatgcgggaagtgagagcactggcctacttcgtgaatggcactgctcaggatgtgattctgtgcgatggctccccaaggggactgctggcatgccagtacaacaccggcaatttcagcgacggattttaccccttcacaaactccagcctggtgaagcagaaatttatcgtgtaccgcgagaacagcgtgaatacaactttcacactgcacaacttcacttttcacaatgaaaccggagccaaccccaatcctagcggggtgcagaacattcagacataccagacccagacagctcagagcgggtactacaacttcaatttttccttcctgtccagctttgtgtacaaggagagcaacttcatgtacggcagctaccacccatcctgcaattttcggctggaaacaatcaacaatgggctgtggttcaactccctgagcgtgtccattgcttacggccctctgcagggcggctgcaaacagagcgtgttttccggaagagccacctgctgctacgcttactcctacggagggccactgctgtgcaagggggtgtacagcggcgagctggatcacaatttcgaatgcggactgctggtgtacgtgaccaaaagcggcggcagcagaatccagactgccaccgagccacccgtgatcacacagcacaactacaacaatattacactgaacacttgcgtggactacaatatctacgggagaactggccagggattcattaccaacgtgacagatagcgctgtgtcctacaattacctggctgacgcaggcctggcaatcctggataccagcggcagcatcgacatttttgtggtgcagtccgagtacggcctgaactactacaaagtgaatccctgcgaagatgtgaaccagcagttcgttgtgagcgggggcaaactggtgggaatcctgacaagccggaatgagactgggtcccagctgctggaaaaccagttctacatcaagatcactaacggaaccgccgccttcgccgccagcatcacagagtccgtggaaaactgcccttacgtgtcctacgggaagttttgcattaaaccagacggcagcatcgccactattgtgcccaagcagctggagcagtttgtggctcctctgctgaacgtgaccgaaaatgtgctgatcccaaacagcttcaatctgacagtgactgatgagtacattcagaccaggatggacaaagtgcagatcaactgcctgcagtacatttgcgggaacagcctggaatgccgcaatctgttccagcagtacggccctgtgtgcgataacatcctgagcgtggtgaacagcgtgggccagaaggaggacatggaactgctgaacttttactccagcactaaacccgccggcttcaacacccctgtgctgagcaatgtgtccaccggagagtttaatatctccctgttcctgaccacaccatccagccccagaaggcgcagctttattgaggatctgctgttcacaagcgtggaatccgtggggctgcccactgatgacgcttacaagaactgcaccgcaggccctctgggattcctgaaagacctggcctgcgctcgcgagtacaatggcctgctggtgctgcctccaatcattacagctgaaatgcagatcctgtacacttccagcctggtggctagcatggcatttggagggatcactgcagccggggcaattcccttcgccacccagctgcaggcaaggatcaaccacctgggcattacacagtccctgctgctgaagaaccaggagaaaatcgctgccagcttcaataaggctattgggcacatgcaggaaggcttccgcagcacttccctggcactgcagcagatccaggatgtggtgaacaagcagtccgccattctgaccgagacaatggctagcctgaacaaaaattttggcgccatctccagcgtgatccaggaaatttaccagcagctggatcccccccaggcaaacgcccaggtggacaggctgattacaggacgcctgtccagcctgagcgtgctggcttccgcaaagcaggcagagtacatccgggtgtcccagcagagagagctggccacacagaagatcaacgaatgcgtgaaaagccagtccattcggtacagcttctgcgggaatggcagacacgtgctgactatccctcagaacgccccaaatggcatcgtgtttattcacttcagctacacccccgactcctttgtgaacgtgacagctatcgtgggattctgcgtgaagccagccaatgcttcccagtacgctattgtgcctgcaaacggaagagggatctttattcaagtgaatggaagctactacatcactgcaagggatatgtacatgcctcgcgccatcaccgctggggacattgtgactctgaccagctgccaggccaactacgtgtccgtgaataaaaccgtgatcactaccttcgtggataacgatgactttgatttcaatgacgagctgagcaagtggtggaacgacacaaaacacgaactgcctgattttgacaagttcaattacactgtgccaatcctggatattgacagcgagatcgataggattcagggagtgatccaggggctgaacgatagcctgattgacctggaaaaactgtccatcctgaagacatacattaaatcctccatcgcttccttcttcttcatcatcggcctgatcatcggactgtttctggtgctgagggtgggcatctacctgtgcatcaagctgaagcacactaagaagaggcagatctacaccgacatcgagatgaacaggctgggcaagtga

SEQ ID NO: 6 - protein, 1140 aa, Artificial Sequence
  chimeric sequence of modified infectious bronchitis virus and vesicular stomatitis virus
MLVTPLLLVTLLCALCSAALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNATTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTAAFAASITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLACAREYNGLLVLPPIITAEMQILYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDPPQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKSSIASFFFIIGLIIGLFLVLRVGIYLCIKLKHTKKRQIYTDIEMNRLGK

SEQ ID NO: 7 - DNA, 3822 bp, Severe Acute Respiratory Syndrome Coronavirus 2
atgttcgtgttcctggtgctgctgcccctggtgtccagccagtgcgtgaacctgaccaccaggacccagctgccaccagcctacaccaacagcttcaccaggggcgtgtactaccccgacaaagtgttcagatcttccgtgctgcacagcacccaggacctgttcctgcccttcttctctaacgtgacctggttccacgccatccacgtgtccggcaccaacggcaccaagaggttcgacaaccccgtgctgcccttcaacgacggcgtgtacttcgccagcaccgagaagtctaacatcatcagaggctggatcttcggcaccaccctggactccaaaacccagagcctgctgatcgtgaacaacgccaccaacgtggtcatcaaggtgtgcgagttccagttctgtaacgaccccttcctgggcgtgtactaccacaagaacaacaaatcttggatggagtccgagttcagggtgtacagctctgccaacaactgcaccttcgagtacgtgagccagcccttcctgatggacctggaaggcaagcagggcaacttcaaaaacctgcgggagttcgtgttcaagaacatcgacggctacttcaagatctacagcaaacacacccccatcaacctggtgcgcgacctgccacagggcttctctgccctggagccactggtggacctgccaatcggcatcaacatcaccaggttccagaccctgctggccctgcacagatcctacctgaccccaggcgactccagctctggatggaccgctggagctgccgcctactacgtgggctacctgcagccccggaccttcctgctgaaatacaacgagaacggaaccatcaccgacgctgtggactgcgctctggacccactgtctgaaaccaagtgtaccctgaaatccttcaccgtggagaagggcatctaccagacctccaacttccgggtgcagcccaccgaaagcatcgtgcgcttccccaacatcaccaacctgtgccccttcggcgaggtgttcaacgctaccaggttcgctagcgtgtacgcttggaaccggaagcgcatcagcaactgcgtggccgactactctgtgctgtacaactccgccagcttctctaccttcaagtgctacggcgtgtcccccaccaaactgaacgacctgtgcttcaccaacgtgtacgccgacagcttcgtgatcaggggcgacgaggtgcgccagatcgctccaggacagaccggcaagatcgctgactacaactacaaactgcccgacgacttcaccggctgcgtgatcgcctggaactctaacaacctggactccaaagtgggcggcaactacaactacctgtacaggctgttcagaaagtctaacctgaaacccttcgagcgggacatcagcaccgaaatctaccaggctggatctaccccatgcaacggagtggagggcttcaactgttacttccccctgcagtcctacggcttccagccaaccaacggagtgggataccagccatacagggtggtggtgctgtctttcgaactgctgcacgctccagctaccgtgtgcggacccaagaaatccaccaacctggtgaagaacaaatgcgtgaacttcaacttcaacggactgaccggaaccggcgtgctgaccgagagcaacaagaaattcctgcccttccagcagttcggccgggacatcgctgacaccaccgacgccgtgcgcgacccccagaccctggaaatcctggacatcaccccctgcagcttcggcggcgtgtctgtgatcaccccaggaaccaacacctccaaccaggtggccgtgctgtaccaggacgtgaactgtaccgaggtgccagtggctatccacgctgaccagctgaccccaacctggagggtgtactctaccggctccaacgtgttccagaccagagctggatgcctgatcggagctgagcacgtgaacaactcctacgaatgcgacatccccatcggcgccggcatctgtgccagctaccagacccagaccaacagcccaaggagagccaggtctgtggcttcccagagcatcatcgcctacaccatgtccctgggcgccgaaaacagcgtggcctacagcaacaactctatcgccatccccaccaacttcaccatcagcgtgaccaccgagatcctgcccgtgtccatgaccaagaccagcgtggactgcaccatgtacatctgtggcgacagcaccgaatgctctaacctgctgctgcagtacggctccttctgtacccagctgaacagagccctgaccggaatcgctgtggagcaggacaaaaacacccaggaagtgttcgcccaggtgaagcagatctacaaaaccccccccatcaaggacttcggcggcttcaacttctcccagatcctgcccgacccctccaagcccagcaaaaggtctttcatcgaggacctgctgttcaacaaggtgaccctggccgacgccggcttcatcaaacagtacggcgactgcctgggcgacatcgctgctagagacctgatctgtgcccagaagttcaacggactgaccgtgctgccaccactgctgaccgacgaaatgatcgctcagtacacctctgccctgctggctggaaccatcacctccggatggaccttcggcgctggagccgccctgcagatccccttcgccatgcagatggcctacagattcaacggcatcggcgtgacccagaacgtgctgtacgagaaccagaagctgatcgccaaccagttcaacagcgccatcggcaaaatccaggactctctgtccagcaccgcttccgccctgggcaaactgcaggacgtggtgaaccagaacgcccaggccctgaacaccctggtgaagcagctgtcttccaacttcggcgccatcagctctgtgctgaacgacatcctgtccaggctggacaaagtggaggccgaagtgcagatcgacaggctgatcaccggcagactgcagagcctgcagacctacgtgacccagcagctgatcagggctgctgaaatcagggcttctgccaacctggctgctaccaagatgtccgagtgcgtgctgggccagagcaagagagtggacttctgtggcaaaggctaccacctgatgtccttcccacagagcgccccacacggagtggtgttcctgcacgtgacctacgtgcccgcccaggagaagaacttcaccaccgctccagctatctgccacgacggcaaagctcacttcccaagggaaggcgtgttcgtgtccaacggcacccactggttcgtgacccagcgcaacttctacgagccccagatcatcaccaccgacaacaccttcgtgagcggcaactgtgacgtggtcatcggaatcgtgaacaacaccgtgtacgacccactgcagccagagctggactctttcaaggaggaactggacaagtacttcaaaaaccacacctccccagacgtggacctgggcgacatctctggcatcaacgcctccgtggtgaacatccagaaggagatcgacaggctgaacgaagtggccaaaaacctgaacgaaagcctgatcgacctgcaggagctgggcaagtacgaacagtacatcaaatggccctggtacatctggctgggcttcatcgccggcctgatcgccatcgtgatggtgaccatcatgctgtgctgtatgacctcctgctgtagctgcctgaagggctgctgttcttgtggctcctgctgtaaattcgacgaggacgactccgaacccgtgctgaagggcgtgaaactgcactacacctga

SEQ ID NO: 8 - protein, 1273 aa, Severe Acute Respiratory Syndrome Coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - DNA, 3783 bp, Artificial Sequence
  chimeric sequence of modified Severe Acute Respiratory Syndrome Coronavirus 2 and vesicular stomatitis virus
atgttcgtgttcctggtgctgctgcccctggtgtccagccagtgcgtgaacctgaccaccagaacccagctgccaccagcctacaccaacagcttcacccggggcgtgtactaccccgacaaagtgttccgctcttccgtgctgcactctacccaggacctgttcctgcccttcttctccaacgtgacctggttccacgccatccacgtgtccggcaccaacggcaccaagaggttcgacaaccccgtgctgcccttcaacgacggcgtgtacttcgcctctaccgagaagtccaacatcatcagaggctggatcttcggcaccaccctggacagcaaaacccagtctctgctgatcgtgaacaacgccaccaacgtggtcatcaaggtgtgcgagttccagttctgtaacgaccccttcctgggcgtgtactaccacaagaacaacaaatcctggatggagagcgagttcagggtgtacagctctgccaacaactgtaccttcgagtacgtgagccagcccttcctgatggacctggaaggcaagcagggcaacttcaaaaacctgcgggagttcgtgttcaagaacatcgacggctacttcaagatctactctaaacacacccccatcaacctggtgcgcgacctgccacagggcttctccgccctggagccactggtggacctgcccatcggcatcaacatcaccaggttccagaccctgctggccctgcaccgctcctacctgaccccaggcgactccagctctggatggaccgctggagctgccgcctactacgtgggctacctgcagcccaggaccttcctgctgaaatacaacgaaaacggaaccatcaccgacgctgtggactgcgctctggacccactgtccgaaaccaagtgtaccctgaaaagcttcaccgtggagaagggcatctaccagaccagcaacttcagggtgcagcccaccgaatctatcgtgagattccccaacatcaccaacctgtgccccttcggcgaggtgttcaacgccaccagattcgccagcgtgtacgcctggaacaggaagagaatctctaactgcgtggccgactactccgtgctgtacaactctgcctccttcagcaccttcaagtgctacggcgtgagccccaccaaactgaacgacctgtgcttcaccaacgtgtacgccgactctttcgtgatcaggggcgacgaggtgagacagatcgctccaggacagaccggcaagatcgctgactacaactacaaactgcccgacgacttcaccggctgcgtgatcgcctggaactccaacaacctggacagcaaagtgggcggcaactacaactacctgtaccggctgttccgcaagagcaacctgaaacccttcgagcgggacatctctaccgaaatctaccaggctggatccaccccatgcaacggagtggagggcttcaactgttacttccccctgcagtcctacggcttccagccaaccaacggagtgggataccagccatacagggtggtggtgctgtccttcgaactgctgcacgctccagctaccgtgtgcggacccaagaaaagcaccaacctggtgaagaacaaatgcgtgaacttcaacttcaacggactgaccggaaccggcgtgctgaccgagagcaacaagaaattcctgcccttccagcagttcggaagggacatcgctgacaccaccgacgccgtgagagacccacagaccctggaaatcctggacatcaccccctgctctttcggcggcgtgtccgtgatcaccccaggaaccaacacctccaaccaggtggccgtgctgtaccaggacgtgaactgtaccgaggtgccagtggctatccacgctgaccagctgaccccaacctggagggtgtacagcaccggctctaacgtgttccagaccagagctggatgcctgatcggagctgagcacgtgaacaacagctacgaatgcgacatccccatcggcgccggcatctgtgcctcttaccagacccagaccaactctccagctgccgcccggtccgtggcttctcagtccatcatcgcctacaccatgagcctgggcgccgaaaactctgtggcctactccaacaacagcatcgccatccccaccaacttcaccatcagcgtgaccaccgagatcctgcccgtgagcatgaccaagacctctgtggactgcaccatgtacatctgtggcgactctaccgaatgctccaacctgctgctgcagtacggctccttctgtacccagctgaaccgcgccctgaccggaatcgctgtggagcaggacaaaaacacccaggaagtgttcgcccaggtgaagcagatctacaaaaccccccccatcaaggacttcggcggcttcaacttctcccagatcctgcccgacccctctaagccctccaaaaggagcttcatcgaggacctgctgttcaacaaggtgaccctggccgacgccggcttcatcaaacagtacggcgactgcctgggcgacatcgctgctagagacctgatctgtgcccagaagttcaacggactgaccgtgctgccaccactgctgaccgacgaaatgatcgctcagtacacctccgccctgctggctggaaccatcaccagcggatggaccttcggcgctggagccgccctgcagatccccttcgccatgcagatggcctacaggttcaacggcatcggcgtgacccagaacgtgctgtacgagaaccagaagctgatcgccaaccagttcaacagcgccatcggcaaaatccaggactccctgtccagcaccgctagcgccctgggcaaactgcaggacgtggtgaaccagaacgcccaggccctgaacaccctggtgaagcagctgtcttccaacttcggcgccatcagctctgtgctgaacgacatcctgtcccggctggacaaagtggaggccgaagtgcagatcgacaggctgatcaccggccgcctgcagtctctgcagacctacgtgacccagcagctgatcagggccgccgaaatcagagcctccgccaacctggccgccaccaagatgagcgagtgcgtgctgggccagtctaagcgcgtggacttctgtggcaaaggctaccacctgatgagcttcccacagtctgccccacacggagtggtgttcctgcacgtgacctacgtgcccgcccaggagaagaacttcaccaccgctccagctatctgccacgacggcaaagctcacttcccaagggaaggcgtgttcgtgagcaacggcacccactggttcgtgacccagcgcaacttctacgagccccagatcatcaccaccgacaacaccttcgtgtccggcaactgtgacgtggtcatcggaatcgtgaacaacaccgtgtacgacccactgcagccagagctggactccttcaaggaggaactggacaagtacttcaaaaaccacaccagcccagacgtggacctgggcgacatctccggcatcaacgccagcgtggtgaacatccagaaggagatcgacaggctgaacgaagtggccaaaaacctgaacgaaagcctgatcgacctgcaggagctgggcaagtacgaacagtacatcaaatccagcatcgcctccttcttcttcatcatcggcctgatcatcggcctgttcctggtgctgagagtgggcatctacctgtgcatcaagctgaaacacaccaagaaacggcagatctacaccgacatcgagatgaaccgcctgggcaagtga

SEQ ID NO: 10 - protein, 1260 aa, Artificial Sequence
  chimeric sequence of modified Severe Acute Respiratory Syndrome Coronavirus 2 and vesicular stomatitis virus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKSSIASFFFIIGLIIGLFLVLRVGIYLCIKLKHTKKRQIYTDIEMNRLGK

SEQ ID NO: 11 - DNA, 3783 bp, Artificial Sequence
  chimeric sequence of modified Severe Acute Respiratory Syndrome Coronavirus 2 and vesicular stomatitis virus
atgttcgtgttcctggtgctgctgcccctggtgtccagccagtgcgtgaacctgaccaccagaacccagctgccaccagcctacaccaacagcttcacccggggcgtgtactaccccgacaaagtgttccgctcttccgtgctgcactctacccaggacctgttcctgcccttcttctccaacgtgacctggttccacgccatccacgtgtccggcaccaacggcaccaagaggttcgacaaccccgtgctgcccttcaacgacggcgtgtacttcgcctctaccgagaagtccaacatcatcagaggctggatcttcggcaccaccctggacagcaaaacccagtctctgctgatcgtgaacaacgccaccaacgtggtcatcaaggtgtgcgagttccagttctgtaacgaccccttcctgggcgtgtactaccacaagaacaacaaatcctggatggagagcgagttcagggtgtacagctctgccaacaactgtaccttcgagtacgtgagccagcccttcctgatggacctggaaggcaagcagggcaacttcaaaaacctgcgggagttcgtgttcaagaacatcgacggctacttcaagatctactctaaacacacccccatcaacctggtgcgcgacctgccacagggcttctccgccctggagccactggtggacctgcccatcggcatcaacatcaccaggttccagaccctgctggccctgcaccgctcctacctgaccccaggcgactccagctctggatggaccgctggagctgccgcctactacgtgggctacctgcagcccaggaccttcctgctgaaatacaacgaaaacggaaccatcaccgacgctgtggactgcgctctggacccactgtccgaaaccaagtgtaccctgaaaagcttcaccgtggagaagggcatctaccagaccagcaacttcagggtgcagcccaccgaatctatcgtgagattccccaacatcaccaacctgtgccccttcggcgaggtgttcaacgccaccagattcgccagcgtgtacgcctggaacaggaaaagaatctctaactgcgtggccgactactccgtgctgtacaactctgcctccttcagcaccttcaagtgctacggcgtgagccccaccaaactgaacgacctgtgcttcaccaacgtgtacgccgactctttcgtgatcaggggcgacgaggtgagacagatcgctccaggacagaccggcaagatcgctgactacaactacaaactgcccgacgacttcaccggctgcgtgatcgcctggaactccaacaacctggacagcaaagtgggcggcaactacaactacctgtaccggctgttccgcaagagcaacctgaaacccttcgagcgggacatctctaccgaaatctaccaggctggatccaccccatgcaacggagtggagggcttcaactgttacttccccctgcagtcctacggcttccagccaaccaacggagtgggataccagccatacagggtggtggtgctgtccttcgaactgctgcacgctccagctaccgtgtgcggacccaagaaaagcaccaacctggtgaagaacaaatgcgtgaacttcaacttcaacggactgaccggaaccggcgtgctgaccgagagcaacaagaaattcctgcccttccagcagttcggaagggacatcgctgacaccaccgacgccgtgagagacccacagaccctggaaatcctggacatcaccccctgctctttcggcggcgtgtccgtgatcaccccaggaaccaacacctccaaccaggtggccgtgctgtaccaggacgtgaactgtaccgaggtgccagtggctatccacgctgaccagctgaccccaacctggagggtgtacagcaccggctctaacgtgttccagaccagagctggatgcctgatcggagctgagcacgtgaacaacagctacgaatgcgacatccccatcggcgccggcatctgtgcctcttaccagacccagaccaactctccagctgccgcccggtccgtggcttctcagtccatcatcgcctacaccatgagcctgggcgccgaaaactctgtggcctactccaacaacagcatcgccatccccaccaacttcaccatcagcgtgaccaccgagatcctgcccgtgagcatgaccaagacctctgtggactgcaccatgtacatctgtggcgactctaccgaatgctccaacctgctgctgcagtacggctccttctgtacccagctgaaccgcgccctgaccggaatcgctgtggagcaggacaaaaacacccaggaagtgttcgcccaggtgaagcagatctacaaaaccccccccatcaaggacttcggcggcttcaacttctcccagatcctgcccgacccctctaagccctccaaaaggagcttcatcgaggacctgctgttcaacaaggtgaccctggccgacgccggcttcatcaaacagtacggcgactgcctgggcgacatcgctgctagagacctgatctgtgcccagaagttcaacggactgaccgtgctgccaccactgctgaccgacgaaatgatcgctcagtacacctccgccctgctggctggaaccatcaccagcggatggaccttcggcgctggagccgccctgcagatccccttcgccatgcagatggcctacaggttcaacggcatcggcgtgacccagaacgtgctgtacgagaaccagaagctgatcgccaaccagttcaacagcgccatcggcaaaatccaggactccctgtccagcaccgctagcgccctgggcaaactgcaggacgtggtgaaccagaacgcccaggccctgaacaccctggtgaagcagctgtcttccaacttcggcgccatcagctctgtgctgaacgacatcctgtccaggctggacccaccagaggctgaagtgcagatcgacaggctgatcaccggccgcctgcagtctctgcagacctacgtgacccagcagctgatcagggccgccgaaatcagagcctccgccaacctggccgccaccaagatgagcgagtgcgtgctgggccagtctaagcgcgtggacttctgtggcaaaggctaccacctgatgagcttcccacagtctgccccacacggagtggtgttcctgcacgtgacctacgtgcccgcccaggagaagaacttcaccaccgctccagctatctgccacgacggcaaagctcacttcccaagggaaggcgtgttcgtgagcaacggcacccactggttcgtgacccagcgcaacttctacgagccccagatcatcaccaccgacaacaccttcgtgtccggcaactgtgacgtggtcatcggaatcgtgaacaacaccgtgtacgacccactgcagccagagctggactccttcaaggaggaactggacaagtacttcaaaaaccacaccagcccagacgtggacctgggcgacatctccggcatcaacgccagcgtggtgaacatccagaaggagatcgacaggctgaacgaagtggccaaaaacctgaacgaaagcctgatcgacctgcaggagctgggcaagtacgaacagtacatcaaatccagcatcgcctccttcttcttcatcatcggcctgatcatcggcctgttcctggtgctgagagtgggcatctacctgtgcatcaagctgaaacacaccaagaaacggcagatctacaccgacatcgagatgaaccgcctgggcaagtga

SEQ ID NO: 12 - protein, 1260 aa, Artificial Sequence
  chimeric sequence of modified Severe Acute Respiratory Syndrome Coronavirus 2 and vesicular stomatitis virus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKSSIASFFFIIGLIIGLFLVLRVGIYLCIKLKHTKKRQIYTDIEMNRLGK

SEQ ID NO: 13 - protein, 4 aa, Severe Acute Respiratory Syndrome Coronavirus 2
RRAR

SEQ ID NO: 14 - protein, 5 aa, Artificial Sequence
  modified furin cleavage site IBV
AAFAA

SEQ ID NO: 15 - protein, 4 aa, Artificial Sequence
  modified furin cleavage site SARS-CoV2
AAAR

SEQ ID NO: 16 - DNA, 4092 bp, Artificial Sequence
  Consensus sequence of 130 BCoV spike protein genes from 2016 - 2021
  CDS (1)..(4092): Consensus BCoV Spike protein
atgtttctgattctgctgattagcctgccaaccgcttttgctgtcatcggggacctgaaatgcaccaccgtgagtattaacgacgtggacacaggcgtgccttccatcagcaccgacaccgtggacgtgaccaacggcctgggcacctactacgtgctggaccgcgtgtacctgaacaccaccctgctgctgaacggctactaccctacctccggcagcacctacagaaacatggccctgaagggcaccctgctgctgtccaccctgtggttcaagccccctttcctgagcgacttcaccaacggcatcttcgccaaggtgaagaacaccaaagtgatcaaggacggcgtgatgtactccgagttccccgccatcaccatcggcagcaccttcgtgaacacctcctacagcgtggtggtgcagcctcacaccaccatcctgggcaacaagctgcagggcttcctggagatctccgtgtgccagtacaccatgtgcgagtaccccaacaccatctgcaaccctaacctgggcaaccagagggtggagctgtggcactgggacacaggcgtggtgagctgcctgtacaagaggaacttcacctacgacgtgaacgccgactacctgtacttccacttctaccaggagggcggcaccttctacgcctacttcaccgacacaggcgtggtgaccaagttcctgttcaacgtgtacctgggcaccgtgctgtcccactactacgtgatgcccctgacctgcaacagcgccctgaccctggagtactgggtgacccctctgaccagcaagcagtacctgctggccttcaaccaggacggcgtgatcttcaacgccgtggactgcaagtccgacttcatgagcgagatcaagtgcaagaccctgtccatcgctcctagcaccggggtgtacgagctgaacggctacaccgtgcagcctatcgccgacgtgtaccggaggatccccaacctgcctgactgcaacatcgaggcctggctgaacgacaagtccgtgcccagccctctgaactgggagcggaagaccttcagcaactgcaacttcaacatgagctccctgatgtccttcatccaggccgacagcttcacctgcaacaacatcgacgccgccaagatctacggcatgtgcttcagctccatcaccatcgacaagttcgccatccccaacggccggaaggtggacctgcagctgggcaacctgggctacctgcagtccttcaactacaggatcgacaccaccgccaccagctgccagctgtactacaacctgcctgctgctaacgtgtccgtgagcaggttcaaccctagcacctggaaccgcagattcggcttcaccgagcagtccgtgttcaagccccagcctgccggcgtgttcaccgaccacgacgtggtgtacgcccagcactgcttcaaggcccccaccaacttctgcccttgcaagctggacggcagcctgtgcgtgggatccggaagcggcatcgacgccggctacaagaacaccggcatcggaacctgccctgctggaaccaactacctgacctgccacaacgctgctcagtgcgactgcctgtgcacccccgaccctatcacctccaaggccaccggcccctacaagtgccctcagaccaagtacctggtgggaatcggagagcactgctccggcctggccatcaagagcgaccactgcggaggaaacccttgcagctgccagcctcaggccttcctgggatggtccgtggacagctgcctgcagggcgaccgctgcaacatcttcgccaacttcatcctgcacgacgtgaactccggcaccacctgcagcaccgacctgcagaagagcaacaccgacatcatcctgggcgtgtgcgtgaactacgacctgtacggcatcaccggccagggcatcttcgtggaggtgaacgccacctactacaactcctggcagaacctgctgtacgacagcaacggcaacctgtacggcttccgcgactacctgaccaaccggaccttcatgatcaggtcctgctacagcggcagagtgagcgccgctttccacgccaacagctccgagcccgccctgctgttccggaacatcaagtgcaactacgtgttcaacaacaccctgtccaggcagctgcagcctatcaactacttcgacagctacctgggctgcgtggtgaacgccgacaactccaccagctccgtggtgcagacctgcgacctgaccgtgggatccggatactgcgtggactacagcaccaagcggaggtcccgcagaagcatcaccaccggctacagattcaccaacttcgagcccttcaccgtgaactccgtgaacgacagcctggagcccgtgggaggcctgtacgagatccagatccctagcgagttcaccatcggcaacatggaggagttcatccagaccagctcccctaaggtgaccatcgactgctccgccttcgtgtgcggcgactacgctgcttgcaagtcccagctggtggagtacggcagcttctgcgacaacatcaacgccatcctgaccgaggtgaacgagctgctggacaccacccagctgcaggtggccaactccctgatgaacggcgtgaccctgagcaccaagctgaaggacggcgtgaacttcaacgtggacgatatcaacttctcccccgtgctgggatgcctgggaagcgactgcaacaaggtgagctcccggtccgccatcgaggacctgctgttctccaaggtgaagctgagcgacgtgggcttcgtggaggcctacaacaactgcacaggcggagctgagatcagggacctgatctgcgtgcagagctacaacggcatcaaggtgctgccccctctgctgtccgagaaccagatcagcggctacaccctggctgccacctccgccagcctgttccctccttggagcgccgccgccggggtgcccttctacctgaacgtgcagtaccgcatcaacggcatcggcgtgaccatggacgtgctgagccagaaccagaagctgatcgccaacgccttcaacaacgctctgggagctatccaggagggattcgacgctaccaacagcgccctggtgaagatccaggctgtggtgaacgctaacgctgaggccctgaacaacctgctgcagcagctgagcaaccgcttcggcgccatcagctccagcctgcaggagatcctgtccagactggacgccctggaggctcaggctcagatcgaccgcctgatcaacggcagactgaccgccctgaacgcctacgtgagccagcagctgtccgacagcaccctggtgaagttctccgccgcccaggccatggagaaggtgaacgagtgcgtgaagagccagtccagccgcatcaacttctgcggcaacggcaaccacatcatctccctggtgcagaacgccccctacggcctgtacttcatccacttcagctacgtgcccaccaagtacgtgaccgccaaggtgtcccctggcctgtgcatcgccggggaccggggcatcgcccctaagagcggctacttcgtgaacgtgaacaacacctggatgttcaccggctccggctactactaccccgagcctatcaccggcaacaacgtggtggtcatgagcacctgcgccgtgaactacaccaaggcccccgacgtgatgctgaacatctccacccccaacctgcctgacttcaaggaggagctggaccagtggttcaagaaccagacctccgtggcccctgacctgagcctggactacatcaacgtgaccttcctggacctgcaggacgagatgaacaggctgcaggaggccatcaaggtgctgaaccagtcctacatcaacctgaaggacatcggcacctacgagtactacgtgaagtggccctggtacgtgtggctgctgatcggctttgccggcgtggccatgctggtgctgctgttcttcatctgctgctgcaccggctgcggcaccagctgcttcaagaagtgcggcggctgctgcgacgattacactggacatcaggaactggtcatcaaaacatcacacgaggactaa

SEQ ID NO: 17 - protein, 1363 aa, Artificial Sequence
  Synthetic Construct
MFLILLISLPTAFAVIGDLKCTTVSINDVDTGVPSISTDTVDVTNGLGTYYVLDRVYLNTTLLLNGYYPTSGSTYRNMALKGTLLLSTLWFKPPFLSDFTNGIFAKVKNTKVIKDGVMYSEFPAITIGSTFVNTSYSVVVQPHTTILGNKLQGFLEISVCQYTMCEYPNTICNPNLGNQRVELWHWDTGVVSCLYKRNFTYDVNADYLYFHFYQEGGTFYAYFTDTGVVTKFLFNVYLGTVLSHYYVMPLTCNSALTLEYWVTPLTSKQYLLAFNQDGVIFNAVDCKSDFMSEIKCKTLSIAPSTGVYELNGYTVQPIADVYRRIPNLPDCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNRRFGFTEQSVFKPQPAGVFTDHDVVYAQHCFKAPTNFCPCKLDGSLCVGSGSGIDAGYKNTGIGTCPAGTNYLTCHNAAQCDCLCTPDPITSKATGPYKCPQTKYLVGIGEHCSGLAIKSDHCGGNPCSCQPQAFLGWSVDSCLQGDRCNIFANFILHDVNSGTTCSTDLQKSNTDIILGVCVNYDLYGITGQGIFVEVNATYYNSWQNLLYDSNGNLYGFRDYLTNRTFMIRSCYSGRVSAAFHANSSEPALLFRNIKCNYVFNNTLSRQLQPINYFDSYLGCVVNADNSTSSVVQTCDLTVGSGYCVDYSTKRRSRRSITTGYRFTNFEPFTVNSVNDSLEPVGGLYEIQIPSEFTIGNMEEFIQTSSPKVTIDCSAFVCGDYAACKSQLVEYGSFCDNINAILTEVNELLDTTQLQVANSLMNGVTLSTKLKDGVNFNVDDINFSPVLGCLGSDCNKVSSRSAIEDLLFSKVKLSDVGFVEAYNNCTGGAEIRDLICVQSYNGIKVLPPLLSENQISGYTLAATSASLFPPWSAAAGVPFYLNVQYRINGIGVTMDVLSQNQKLIANAFNNALGAIQEGFDATNSALVKIQAVVNANAEALNNLLQQLSNRFGAISSSLQEILSRLDALEAQAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMFTGSGYYYPEPITGNNVVVMSTCAVNYTKAPDVMLNISTPNLPDFKEELDQWFKNQTSVAPDLSLDYINVTFLDLQDEMNRLQEAIKVLNQSYINLKDIGTYEYYVKWPWYVWLLIGFAGVAMLVLLFFICCCTGCGTSCFKKCGGCCDDYTGHQELVIKTSHED

SEQ ID NO: 18 - DNA, 3390 bp, porcine enteric alphacoronavirus
  CDS (1)..(3390): SADS-CoV Spike protein
atgaagctgtttaccgtgtttaccctgctcgcctcaatcagagtgctctatggctgtgaatccgtggactttaacctgtttaataccatcttctccacccacaggggcctgtccaacaccaccagcgtgatcaccggcgcctaccccagcaccaacaagtccgactggagctgcaacaccaggaccggacacctgtccggaagcggattcggaatcggcctgtacgtgcagacccccagagagcagtaccagtacgacggatccggagctggaggatacaccatcgccgtgagcccaatccacgtgaccaacctgacctgggagctgtggatccacagaaagtggggcgtgaactccgtggtgaccgtgaggctgtgcagatggtggcagttcatgagcttcaactccaccagccacgctgctgacgctggacccaccaacgccttcgagtgcctgatcaacggcagctacccaacccaccgcaacaccggctacatgttcggcgtgacctggtacaacgacctggtgcggatcgtgttccccccaaccgtgctggagatgcagctggacggcctgcagtgggagagggtgcagttcaactccccagtgaacgctggacacgctacccggttcaacgtggtgaaggacatctccaccgtgctggtggagacaaactccggcggcagcgtgttcaggtacagctactgcgccgacggcttcgtgaacggcctgcagtgcaagctgagactgttcgacatccccccaggcgtgtactccaacagcgaggtggagtacccaaccgccctgtacaccgtggtgcacaacatgtccgcctgccccgagaggccagacagctactgcggctccaacagctgccccttcaagagagccgtgttcagcaactgcatcgtgaactacaccacctgggtgaacccagaccagcgcgacttccagcacctgatcctgcccaacggcaagttcaacccattcaccgagtgcaacggcctgaacaggatcgtggacggatgcgtgccaggattcgtgctgagggtgggaaggggcaaggccgtgaaccggaccatcgtgaccccctacctgaagccatacgagtgcttcggctggtcctggaacgacaaccaggacagcatctacgactggtggatcgccgacttcgtgtccaccggagccttcgtgtgcgagagcaacccagaggctccaaagaccggcgtgtgcgtgacctacaccgtggagaaggtgaccttccagggcgtgctgtacgagtccaacttcaccttcgcccagtactacaacctgctgtacgtgggcagccagctgagatacgtgagaatcctgggcaaggtgtacgaggtgtccagctgtttcgaggccagctacgacgtgctgtaccgcaacaaccagtccttcggcctgctgtaccggagcttcgactgcaaccagctgcacatcaagtccgccagattcgtggacaggctgctgccaagccacaacggaaccgccaccgtgctgggctgcctgttcaacgcctcctacgcccccaacgacaccatggtgaactgcaccaacccactgggcgacggattctgcgctgacctgctgggaaacgtggccgtgcgcaggatgaccttcgagaagcacgacaccacctacgtggcccccgtgaccaacgagaggtacaccgagatgccactggaccaccagctgatcctgaccgagcagttcctgcagaccaccatgccaaagttctccgtgagctgcgagacatacatctgcgacgtgagcaaggcctgcaagaacctgctgttcaggtacggcggcttctgccagaaggtggaggccgacatcagaggagctggcatcctgctggacggcgacgtgtccagcctgtactccaccatcgctgctaagacctccagcgtggtgccaaccaccgacaggttcaacgtgagccagttcttcctgccaaagacccagtccagcgccaacaagtacgagtccagaagcgccatcgaggacctgctgttctccaagatcgagacaaccggccccggcttctacggcgactactacaactgcaagaagaacgccatccaggacctgacctgcgcccagtaccacaacggcatcctggtcatccccccaatcatggacgccgagacactgggaatgtacggaggaatcgctgccgcctccgtgaccctgggaatcttcggaggacaggctggaatggctacctggagcgtggccatggccggccggctgaacgctctgggagtggtgcagaacgctctggtggacgacgtgaacaagctggccaacggcttcaaccagctgaccgcctccgtgagcaagctggccctgaccacctccagcgccctgcaggctatccaggctgtggtgaaccagaacgctgctcaggtggagtccctggtgagcggcatcaccgagaacttcggcgccatctccaccaacttcaaagtgatcagccagcgcctggacaagctggaggctgacgtgcagatggaccgcctgatcaacggccggatgaacgtgctgcagctgttcgtgaccaactacaagctgaagatcgccgagctgaggaacacccacagatacgtgcagtccctgatcaacgagtgcgtgtacgctcagagcctgaggaacggattctgcggacagggcctgcacgtgctgtccctgatgcagaacgcccccagcggcatcatgttcttccactacagcctgatccccaacaacaccatcaccgtgaagaccaccccaggcctgtgcgagtccgacgagctgggaagcaagtgcatcgtggctaaggacggcgtgctggtgtccgccaacctgagctactggcagtggtccccccgcaacctgtacaagccagagaacctgaccttcgccaacgtgatcgccgtgagcaggggagctaactacaccaccctgaacaagaccttcgacatccccgagctgaactccaccttcccaatcgaggaggagttccgcgagtacttccagaacatgtccagcgagctgcaggtgctgaagaacctgaccgccgacatgtccaagctgaacatcagcgccgagatccagctgatcaacgagatcgcccacaacgtgtccaacatgagggtggaggtggagaagttccagagatacgtgaactacgtgaagtgggcctggtggcagtggctgctgatcttcatcgccctgaccctgctggctggcctgatgctgtggtgctgcctggccaccggctgctgcggcatgtgcggatgcctggctgctacctgcgcttcctgctgcgactgcaggggaaccaagctgcagagctacgagatcgagaaggtccatgtccag

SEQ ID NO: 19 - protein, 1130 aa, porcine enteric alphacoronavirus
MKLFTVFTLLASIRVLYGCESVDFNLFNTIFSTHRGLSNTTSVITGAYPSTNKSDWSCNTRTGHLSGSGFGIGLYVQTPREQYQYDGSGAGGYTIAVSPIHVTNLTWELWIHRKWGVNSVVTVRLCRWWQFMSFNSTSHAADAGPTNAFECLINGSYPTHRNTGYMFGVTWYNDLVRIVFPPTVLEMQLDGLQWERVQFNSPVNAGHATRFNVVKDISTVLVETNSGGSVFRYSYCADGFVNGLQCKLRLFDIPPGVYSNSEVEYPTALYTVVHNMSACPERPDSYCGSNSCPFKRAVFSNCIVNYTTWVNPDQRDFQHLILPNGKFNPFTECNGLNRIVDGCVPGFVLRVGRGKAVNRTIVTPYLKPYECFGWSWNDNQDSIYDWWIADFVSTGAFVCESNPEAPKTGVCVTYTVEKVTFQGVLYESNFTFAQYYNLLYVGSQLRYVRILGKVYEVSSCFEASYDVLYRNNQSFGLLYRSFDCNQLHIKSARFVDRLLPSHNGTATVLGCLFNASYAPNDTMVNCTNPLGDGFCADLLGNVAVRRMTFEKHDTTYVAPVTNERYTEMPLDHQLILTEQFLQTTMPKFSVSCETYICDVSKACKNLLFRYGGFCQKVEADIRGAGILLDGDVSSLYSTIAAKTSSVVPTTDRFNVSQFFLPKTQSSANKYESRSAIEDLLFSKIETTGPGFYGDYYNCKKNAIQDLTCAQYHNGILVIPPIMDAETLGMYGGIAAASVTLGIFGGQAGMATWSVAMAGRLNALGVVQNALVDDVNKLANGFNQLTASVSKLALTTSSALQAIQAVVNQNAAQVESLVSGITENFGAISTNFKVISQRLDKLEADVQMDRLINGRMNVLQLFVTNYKLKIAELRNTHRYVQSLINECVYAQSLRNGFCGQGLHVLSLMQNAPSGIMFFHYSLIPNNTITVKTTPGLCESDELGSKCIVAKDGVLVSANLSYWQWSPRNLYKPENLTFANVIAVSRGANYTTLNKTFDIPELNSTFPIEEEFREYFQNMSSELQVLKNLTADMSKLNISAEIQLINEIAHNVSNMRVEVEKFQRYVNYVKWAWWQWLLIFIALTLLAGLMLWCCLATGCCGMCGCLAATCASCCDCRGTKLQSYEIEKVHVQ

SEQ ID NO: 20 - DNA, 4092 bp, Artificial Sequence
  BCoV consensus Spike with furin site mutation
atgtttctgattctgctgattagcctgccaaccgcttttgctgtcatcggggacctgaaatgcaccaccgtgagtattaacgacgtggacacaggcgtgccttccatcagcaccgacaccgtggacgtgaccaacggcctgggcacctactacgtgctggaccgcgtgtacctgaacaccaccctgctgctgaacggctactaccctacctccggcagcacctacagaaacatggccctgaagggcaccctgctgctgtccaccctgtggttcaagccccctttcctgagcgacttcaccaacggcatcttcgccaaggtgaagaacaccaaagtgatcaaggacggcgtgatgtactccgagttccccgccatcaccatcggcagcaccttcgtgaacacctcctacagcgtggtggtgcagcctcacaccaccatcctgggcaacaagctgcagggcttcctggagatctccgtgtgccagtacaccatgtgcgagtaccccaacaccatctgcaaccctaacctgggcaaccagagggtggagctgtggcactgggacacaggcgtggtgagctgcctgtacaagaggaacttcacctacgacgtgaacgccgactacctgtacttccacttctaccaggagggcggcaccttctacgcctacttcaccgacacaggcgtggtgaccaagttcctgttcaacgtgtacctgggcaccgtgctgtcccactactacgtgatgcccctgacctgcaacagcgccctgaccctggagtactgggtgacccctctgaccagcaagcagtacctgctggccttcaaccaggacggcgtgatcttcaacgccgtggactgcaagtccgacttcatgagcgagatcaagtgcaagaccctgtccatcgctcctagcaccggggtgtacgagctgaacggctacaccgtgcagcctatcgccgacgtgtaccggaggatccccaacctgcctgactgcaacatcgaggcctggctgaacgacaagtccgtgcccagccctctgaactgggagcggaagaccttcagcaactgcaacttcaacatgagctccctgatgtccttcatccaggccgacagcttcacctgcaacaacatcgacgccgccaagatctacggcatgtgcttcagctccatcaccatcgacaagttcgccatccccaacggccggaaggtggacctgcagctgggcaacctgggctacctgcagtccttcaactacaggatcgacaccaccgccaccagctgccagctgtactacaacctgcctgctgctaacgtgtccgtgagcaggttcaaccctagcacctggaaccgcagattcggcttcaccgagcagtccgtgttcaagccccagcctgccggcgtgttcaccgaccacgacgtggtgtacgcccagcactgcttcaaggcccccaccaacttctgcccttgcaagctggacggcagcctgtgcgtgggatccggaagcggcatcgacgccggctacaagaacaccggcatcggaacctgccctgctggaaccaactacctgacctgccacaacgctgctcagtgcgactgcctgtgcacccccgaccctatcacctccaaggccaccggcccctacaagtgccctcagaccaagtacctggtgggaatcggagagcactgctccggcctggccatcaagagcgaccactgcggaggaaacccttgcagctgccagcctcaggccttcctgggatggtccgtggacagctgcctgcagggcgaccgctgcaacatcttcgccaacttcatcctgcacgacgtgaactccggcaccacctgcagcaccgacctgcagaagagcaacaccgacatcatcctgggcgtgtgcgtgaactacgacctgtacggcatcaccggccagggcatcttcgtggaggtgaacgccacctactacaactcctggcagaacctgctgtacgacagcaacggcaacctgtacggcttccgcgactacctgaccaaccggaccttcatgatcaggtcctgctacagcggcagagtgagcgccgctttccacgccaacagctccgagcccgccctgctgttccggaacatcaagtgcaactacgtgttcaacaacaccctgtccaggcagctgcagcctatcaactacttcgacagctacctgggctgcgtggtgaacgccgacaactccaccagctccgtggtgcagacctgcgacctgaccgtgggatccggatactgcgtggactacagcaccaaggccgcctccgccgccagcatcaccaccggctacagattcaccaacttcgagcccttcaccgtgaactccgtgaacgacagcctggagcccgtgggaggcctgtacgagatccagatccctagcgagttcaccatcggcaacatggaggagttcatccagaccagctcccctaaggtgaccatcgactgctccgccttcgtgtgcggcgactacgctgcttgcaagtcccagctggtggagtacggcagcttctgcgacaacatcaacgccatcctgaccgaggtgaacgagctgctggacaccacccagctgcaggtggccaactccctgatgaacggcgtgaccctgagcaccaagctgaaggacggcgtgaacttcaacgtggacgatatcaacttctcccccgtgctgggatgcctgggaagcgactgcaacaaggtgagctcccggtccgccatcgaggacctgctgttctccaaggtgaagctgagcgacgtgggcttcgtggaggcctacaacaactgcacaggcggagctgagatcagggacctgatctgcgtgcagagctacaacggcatcaaggtgctgccccctctgctgtccgagaaccagatcagcggctacaccctggctgccacctccgccagcctgttccctccttggagcgccgccgccggggtgcccttctacctgaacgtgcagtaccgcatcaacggcatcggcgtgaccatggacgtgctgagccagaaccagaagctgatcgccaacgccttcaacaacgctctgggagctatccaggagggattcgacgctaccaacagcgccctggtgaagatccaggctgtggtgaacgctaacgctgaggccctgaacaacctgctgcagcagctgagcaaccgcttcggcgccatcagctccagcctgcaggagatcctgtccagactggacgccctggaggctcaggctcagatcgaccgcctgatcaacggcagactgaccgccctgaacgcctacgtgagccagcagctgtccgacagcaccctggtgaagttctccgccgcccaggccatggagaaggtgaacgagtgcgtgaagagccagtccagccgcatcaacttctgcggcaacggcaaccacatcatctccctggtgcagaacgccccctacggcctgtacttcatccacttcagctacgtgcccaccaagtacgtgaccgccaaggtgtcccctggcctgtgcatcgccggggaccggggcatcgcccctaagagcggctacttcgtgaacgtgaacaacacctggatgttcaccggctccggctactactaccccgagcctatcaccggcaacaacgtggtggtcatgagcacctgcgccgtgaactacaccaaggcccccgacgtgatgctgaacatctccacccccaacctgcctgacttcaaggaggagctggaccagtggttcaagaaccagacctccgtggcccctgacctgagcctggactacatcaacgtgaccttcctggacctgcaggacgagatgaacaggctgcaggaggccatcaaggtgctgaaccagtcctacatcaacctgaaggacatcggcacctacgagtactacgtgaagtggccctggtacgtgtggctgctgatcggctttgccggcgtggccatgctggtgctgctgttcttcatctgctgctgcaccggctgcggcaccagctgcttcaagaagtgcggcggctgctgcgacgattacactggacatcaggaactggtcatcaaaacatcacacgaggactaa

SEQ ID NO: 21 - DNA, 4092 bp, Artificial Sequence
  BCoV consensus Spike with mutated furin site and 2P mutation
atgtttctgattctgctgattagcctgccaaccgcttttgctgtcatcggggacctgaaatgcaccaccgtgagtattaacgacgtggacacaggcgtgccttccatcagcaccgacaccgtggacgtgaccaacggcctgggcacctactacgtgctggaccgcgtgtacctgaacaccaccctgctgctgaacggctactaccctacctccggcagcacctacagaaacatggccctgaagggcaccctgctgctgtccaccctgtggttcaagccccctttcctgagcgacttcaccaacggcatcttcgccaaggtgaagaacaccaaagtgatcaaggacggcgtgatgtactccgagttccccgccatcaccatcggcagcaccttcgtgaacacctcctacagcgtggtggtgcagcctcacaccaccatcctgggcaacaagctgcagggcttcctggagatctccgtgtgccagtacaccatgtgcgagtaccccaacaccatctgcaaccctaacctgggcaaccagagggtggagctgtggcactgggacacaggcgtggtgagctgcctgtacaagaggaacttcacctacgacgtgaacgccgactacctgtacttccacttctaccaggagggcggcaccttctacgcctacttcaccgacacaggcgtggtgaccaagttcctgttcaacgtgtacctgggcaccgtgctgtcccactactacgtgatgcccctgacctgcaacagcgccctgaccctggagtactgggtgacccctctgaccagcaagcagtacctgctggccttcaaccaggacggcgtgatcttcaacgccgtggactgcaagtccgacttcatgagcgagatcaagtgcaagaccctgtccatcgctcctagcaccggggtgtacgagctgaacggctacaccgtgcagcctatcgccgacgtgtaccggaggatccccaacctgcctgactgcaacatcgaggcctggctgaacgacaagtccgtgcccagccctctgaactgggagcggaagaccttcagcaactgcaacttcaacatgagctccctgatgtccttcatccaggccgacagcttcacctgcaacaacatcgacgccgccaagatctacggcatgtgcttcagctccatcaccatcgacaagttcgccatccccaacggccggaaggtggacctgcagctgggcaacctgggctacctgcagtccttcaactacaggatcgacaccaccgccaccagctgccagctgtactacaacctgcctgctgctaacgtgtccgtgagcaggttcaaccctagcacctggaaccgcagattcggcttcaccgagcagtccgtgttcaagccccagcctgccggcgtgttcaccgaccacgacgtggtgtacgcccagcactgcttcaaggcccccaccaacttctgcccttgcaagctggacggcagcctgtgcgtgggatccggaagcggcatcgacgccggctacaagaacaccggcatcggaacctgccctgctggaaccaactacctgacctgccacaacgctgctcagtgcgactgcctgtgcacccccgaccctatcacctccaaggccaccggcccctacaagtgccctcagaccaagtacctggtgggaatcggagagcactgctccggcctggccatcaagagcgaccactgcggaggaaacccttgcagctgccagcctcaggccttcctgggatggtccgtggacagctgcctgcagggcgaccgctgcaacatcttcgccaacttcatcctgcacgacgtgaactccggcaccacctgcagcaccgacctgcagaagagcaacaccgacatcatcctgggcgtgtgcgtgaactacgacctgtacggcatcaccggccagggcatcttcgtggaggtgaacgccacctactacaactcctggcagaacctgctgtacgacagcaacggcaacctgtacggcttccgcgactacctgaccaaccggaccttcatgatcaggtcctgctacagcggcagagtgagcgccgctttccacgccaacagctccgagcccgccctgctgttccggaacatcaagtgcaactacgtgttcaacaacaccctgtccaggcagctgcagcctatcaactacttcgacagctacctgggctgcgtggtgaacgccgacaactccaccagctccgtggtgcagacctgcgacctgaccgtgggatccggatactgcgtggactacagcaccaaggccgcctccgccgccagcatcaccaccggctacagattcaccaacttcgagcccttcaccgtgaactccgtgaacgacagcctggagcccgtgggaggcctgtacgagatccagatccctagcgagttcaccatcggcaacatggaggagttcatccagaccagctcccctaaggtgaccatcgactgctccgccttcgtgtgcggcgactacgctgcttgcaagtcccagctggtggagtacggcagcttctgcgacaacatcaacgccatcctgaccgaggtgaacgagctgctggacaccacccagctgcaggtggccaactccctgatgaacggcgtgaccctgagcaccaagctgaaggacggcgtgaacttcaacgtggacgatatcaacttctcccccgtgctgggatgcctgggaagcgactgcaacaaggtgagctcccggtccgccatcgaggacctgctgttctccaaggtgaagctgagcgacgtgggcttcgtggaggcctacaacaactgcacaggcggagctgagatcagggacctgatctgcgtgcagagctacaacggcatcaaggtgctgccccctctgctgtccgagaaccagatcagcggctacaccctggctgccacctccgccagcctgttccctccttggagcgccgccgccggggtgcccttctacctgaacgtgcagtaccgcatcaacggcatcggcgtgaccatggacgtgctgagccagaaccagaagctgatcgccaacgccttcaacaacgctctgggagctatccaggagggattcgacgctaccaacagcgccctggtgaagatccaggctgtggtgaacgctaacgctgaggccctgaacaacctgctgcagcagctgagcaaccgcttcggcgccatcagctccagcctgcaggagatcctgtccagactggacccccccgaggctcaggctcagatcgaccgcctgatcaacggcagactgaccgccctgaacgcctacgtgagccagcagctgtccgacagcaccctggtgaagttctccgccgcccaggccatggagaaggtgaacgagtgcgtgaagagccagtccagccgcatcaacttctgcggcaacggcaaccacatcatctccctggtgcagaacgccccctacggcctgtacttcatccacttcagctacgtgcccaccaagtacgtgaccgccaaggtgtcccctggcctgtgcatcgccggggaccggggcatcgcccctaagagcggctacttcgtgaacgtgaacaacacctggatgttcaccggctccggctactactaccccgagcctatcaccggcaacaacgtggtggtcatgagcacctgcgccgtgaactacaccaaggcccccgacgtgatgctgaacatctccacccccaacctgcctgacttcaaggaggagctggaccagtggttcaagaaccagacctccgtggcccctgacctgagcctggactacatcaacgtgaccttcctggacctgcaggacgagatgaacaggctgcaggaggccatcaaggtgctgaaccagtcctacatcaacctgaaggacatcggcacctacgagtactacgtgaagtggccctggtacgtgtggctgctgatcggctttgccggcgtggccatgctggtgctgctgttcttcatctgctgctgcaccggctgcggcaccagctgcttcaagaagtgcggcggctgctgcgacgattacactggacatcaggaactggtcatcaaaacatcacacgaggactaa

SEQ ID NO: 22 - DNA, 4032 bp, Artificial Sequence
  BCoV consensus Spike protein with furin site mutation and Influenza virus HA gene TMD-CTD region
atgtttctgattctgctgattagcctgccaaccgcttttgctgtcatcggggacctgaaatgcaccaccgtgagtattaacgacgtggacacaggcgtgccttccatcagcaccgacaccgtggacgtgaccaacggcctgggcacctactacgtgctggaccgcgtgtacctgaacaccaccctgctgctgaacggctactaccctacctccggcagcacctacagaaacatggccctgaagggcaccctgctgctgtccaccctgtggttcaagccccctttcctgagcgacttcaccaacggcatcttcgccaaggtgaagaacaccaaagtgatcaaggacggcgtgatgtactccgagttccccgccatcaccatcggcagcaccttcgtgaacacctcctacagcgtggtggtgcagcctcacaccaccatcctgggcaacaagctgcagggcttcctggagatctccgtgtgccagtacaccatgtgcgagtaccccaacaccatctgcaaccctaacctgggcaaccagagggtggagctgtggcactgggacacaggcgtggtgagctgcctgtacaagaggaacttcacctacgacgtgaacgccgactacctgtacttccacttctaccaggagggcggcaccttctacgcctacttcaccgacacaggcgtggtgaccaagttcctgttcaacgtgtacctgggcaccgtgctgtcccactactacgtgatgcccctgacctgcaacagcgccctgaccctggagtactgggtgacccctctgaccagcaagcagtacctgctggccttcaaccaggacggcgtgatcttcaacgccgtggactgcaagtccgacttcatgagcgagatcaagtgcaagaccctgtccatcgctcctagcaccggggtgtacgagctgaacggctacaccgtgcagcctatcgccgacgtgtaccggaggatccccaacctgcctgactgcaacatcgaggcctggctgaacgacaagtccgtgcccagccctctgaactgggagcggaagaccttcagcaactgcaacttcaacatgagctccctgatgtccttcatccaggccgacagcttcacctgcaacaacatcgacgccgccaagatctacggcatgtgcttcagctccatcaccatcgacaagttcgccatccccaacggccggaaggtggacctgcagctgggcaacctgggctacctgcagtccttcaactacaggatcgacaccaccgccaccagctgccagctgtactacaacctgcctgctgctaacgtgtccgtgagcaggttcaaccctagcacctggaaccgcagattcggcttcaccgagcagtccgtgttcaagccccagcctgccggcgtgttcaccgaccacgacgtggtgtacgcccagcactgcttcaaggcccccaccaacttctgcccttgcaagctggacggcagcctgtgcgtgggatccggaagcggcatcgacgccggctacaagaacaccggcatcggaacctgccctgctggaaccaactacctgacctgccacaacgctgctcagtgcgactgcctgtgcacccccgaccctatcacctccaaggccaccggcccctacaagtgccctcagaccaagtacctggtgggaatcggagagcactgctccggcctggccatcaagagcgaccactgcggaggaaacccttgcagctgccagcctcaggccttcctgggatggtccgtggacagctgcctgcagggcgaccgctgcaacatcttcgccaacttcatcctgcacgacgtgaactccggcaccacctgcagcaccgacctgcagaagagcaacaccgacatcatcctgggcgtgtgcgtgaactacgacctgtacggcatcaccggccagggcatcttcgtggaggtgaacgccacctactacaactcctggcagaacctgctgtacgacagcaacggcaacctgtacggcttccgcgactacctgaccaaccggaccttcatgatcaggtcctgctacagcggcagagtgagcgccgctttccacgccaacagctccgagcccgccctgctgttccggaacatcaagtgcaactacgtgttcaacaacaccctgtccaggcagctgcagcctatcaactacttcgacagctacctgggctgcgtggtgaacgccgacaactccaccagctccgtggtgcagacctgcgacctgaccgtgggatccggatactgcgtggactacagcaccaaggccgcctccgccgccagcatcaccaccggctacagattcaccaacttcgagcccttcaccgtgaactccgtgaacgacagcctggagcccgtgggaggcctgtacgagatccagatccctagcgagttcaccatcggcaacatggaggagttcatccagaccagctcccctaaggtgaccatcgactgctccgccttcgtgtgcggcgactacgctgcttgcaagtcccagctggtggagtacggcagcttctgcgacaacatcaacgccatcctgaccgaggtgaacgagctgctggacaccacccagctgcaggtggccaactccctgatgaacggcgtgaccctgagcaccaagctgaaggacggcgtgaacttcaacgtggacgatatcaacttctcccccgtgctgggatgcctgggaagcgactgcaacaaggtgagctcccggtccgccatcgaggacctgctgttctccaaggtgaagctgagcgacgtgggcttcgtggaggcctacaacaactgcacaggcggagctgagatcagggacctgatctgcgtgcagagctacaacggcatcaaggtgctgccccctctgctgtccgagaaccagatcagcggctacaccctggctgccacctccgccagcctgttccctccttggagcgccgccgccggggtgcccttctacctgaacgtgcagtaccgcatcaacggcatcggcgtgaccatggacgtgctgagccagaaccagaagctgatcgccaacgccttcaacaacgctctgggagctatccaggagggattcgacgctaccaacagcgccctggtgaagatccaggctgtggtgaacgctaacgctgaggccctgaacaacctgctgcagcagctgagcaaccgcttcggcgccatcagctccagcctgcaggagatcctgtccagactggacgccctggaggctcaggctcagatcgaccgcctgatcaacggcagactgaccgccctgaacgcctacgtgagccagcagctgtccgacagcaccctggtgaagttctccgccgcccaggccatggagaaggtgaacgagtgcgtgaagagccagtccagccgcatcaacttctgcggcaacggcaaccacatcatctccctggtgcagaacgccccctacggcctgtacttcatccacttcagctacgtgcccaccaagtacgtgaccgccaaggtgtcccctggcctgtgcatcgccggggaccggggcatcgcccctaagagcggctacttcgtgaacgtgaacaacacctggatgttcaccggctccggctactactaccccgagcctatcaccggcaacaacgtggtggtcatgagcacctgcgccgtgaactacaccaaggcccccgacgtgatgctgaacatctccacccccaacctgcctgacttcaaggaggagctggaccagtggttcaagaaccagacctccgtggcccctgacctgagcctggactacatcaacgtgaccttcctggacctgcaggacgagatgaacaggctgcaggaggccatcaaggtgctgaaccagtcctacatcaacctgaaggacatcggcacctacgagtactacgtgaagtggcccctggccatctacagcaccgtggctagctccctggtgctgctggtgagcctgggagccatctccttctggatgtgcagcaacggctccctgcagtgccggatctgcatctaa

SEQ ID NO: 23 - DNA, 4071 bp, Artificial Sequence
  BCoV consensus Spike protein with furin site mutation and VSV G protein TMD-CTD region
atgtttctgattctgctgattagcctgccaaccgcttttgctgtcatcggggacctgaaatgcaccaccgtgagtattaacgacgtggacacaggcgtgccttccatcagcaccgacaccgtggacgtgaccaacggcctgggcacctactacgtgctggaccgcgtgtacctgaacaccaccctgctgctgaacggctactaccctacctccggcagcacctacagaaacatggccctgaagggcaccctgctgctgtccaccctgtggttcaagccccctttcctgagcgacttcaccaacggcatcttcgccaaggtgaagaacaccaaagtgatcaaggacggcgtgatgtactccgagttccccgccatcaccatcggcagcaccttcgtgaacacctcctacagcgtggtggtgcagcctcacaccaccatcctgggcaacaagctgcagggcttcctggagatctccgtgtgccagtacaccatgtgcgagtaccccaacaccatctgcaaccctaacctgggcaaccagagggtggagctgtggcactgggacacaggcgtggtgagctgcctgtacaagaggaacttcacctacgacgtgaacgccgactacctgtacttccacttctaccaggagggcggcaccttctacgcctacttcaccgacacaggcgtggtgaccaagttcctgttcaacgtgtacctgggcaccgtgctgtcccactactacgtgatgcccctgacctgcaacagcgccctgaccctggagtactgggtgacccctctgaccagcaagcagtacctgctggccttcaaccaggacggcgtgatcttcaacgccgtggactgcaagtccgacttcatgagcgagatcaagtgcaagaccctgtccatcgctcctagcaccggggtgtacgagctgaacggctacaccgtgcagcctatcgccgacgtgtaccggaggatccccaacctgcctgactgcaacatcgaggcctggctgaacgacaagtccgtgcccagccctctgaactgggagcggaagaccttcagcaactgcaacttcaacatgagctccctgatgtccttcatccaggccgacagcttcacctgcaacaacatcgacgccgccaagatctacggcatgtgcttcagctccatcaccatcgacaagttcgccatccccaacggccggaaggtggacctgcagctgggcaacctgggctacctgcagtccttcaactacaggatcgacaccaccgccaccagctgccagctgtactacaacctgcctgctgctaacgtgtccgtgagcaggttcaaccctagcacctggaaccgcagattcggcttcaccgagcagtccgtgttcaagccccagcctgccggcgtgttcaccgaccacgacgtggtgtacgcccagcactgcttcaaggcccccaccaacttctgcccttgcaagctggacggcagcctgtgcgtgggatccggaagcggcatcgacgccggctacaagaacaccggcatcggaacctgccctgctggaaccaactacctgacctgccacaacgctgctcagtgcgactgcctgtgcacccccgaccctatcacctccaaggccaccggcccctacaagtgccctcagaccaagtacctggtgggaatcggagagcactgctccggcctggccatcaagagcgaccactgcggaggaaacccttgcagctgccagcctcaggccttcctgggatggtccgtggacagctgcctgcagggcgaccgctgcaacatcttcgccaacttcatcctgcacgacgtgaactccggcaccacctgcagcaccgacctgcagaagagcaacaccgacatcatcctgggcgtgtgcgtgaactacgacctgtacggcatcaccggccagggcatcttcgtggaggtgaacgccacctactacaactcctggcagaacctgctgtacgacagcaacggcaacctgtacggcttccgcgactacctgaccaaccggaccttcatgatcaggtcctgctacagcggcagagtgagcgccgctttccacgccaacagctccgagcccgccctgctgttccggaacatcaagtgcaactacgtgttcaacaacaccctgtccaggcagctgcagcctatcaactacttcgacagctacctgggctgcgtggtgaacgccgacaactccaccagctccgtggtgcagacctgcgacctgaccgtgggatccggatactgcgtggactacagcaccaaggccgcctccgccgccagcatcaccaccggctacagattcaccaacttcgagcccttcaccgtgaactccgtgaacgacagcctggagcccgtgggaggcctgtacgagatccagatccctagcgagttcaccatcggcaacatggaggagttcatccagaccagctcccctaaggtgaccatcgactgctccgccttcgtgtgcggcgactacgctgcttgcaagtcccagctggtggagtacggcagcttctgcgacaacatcaacgccatcctgaccgaggtgaacgagctgctggacaccacccagctgcaggtggccaactccctgatgaacggcgtgaccctgagcaccaagctgaaggacggcgtgaacttcaacgtggacgatatcaacttctcccccgtgctgggatgcctgggaagcgactgcaacaaggtgagctcccggtccgccatcgaggacctgctgttctccaaggtgaagctgagcgacgtgggcttcgtggaggcctacaacaactgcacaggcggagctgagatcagggacctgatctgcgtgcagagctacaacggcatcaaggtgctgccccctctgctgtccgagaaccagatcagcggctacaccctggctgccacctccgccagcctgttccctccttggagcgccgccgccggggtgcccttctacctgaacgtgcagtaccgcatcaacggcatcggcgtgaccatggacgtgctgagccagaaccagaagctgatcgccaacgccttcaacaacgctctgggagctatccaggagggattcgacgctaccaacagcgccctggtgaagatccaggctgtggtgaacgctaacgctgaggccctgaacaacctgctgcagcagctgagcaaccgcttcggcgccatcagctccagcctgcaggagatcctgtccagactggacgccctggaggctcaggctcagatcgaccgcctgatcaacggcagactgaccgccctgaacgcctacgtgagccagcagctgtccgacagcaccctggtgaagttctccgccgcccaggccatggagaaggtgaacgagtgcgtgaagagccagtccagccgcatcaacttctgcggcaacggcaaccacatcatctccctggtgcagaacgccccctacggcctgtacttcatccacttcagctacgtgcccaccaagtacgtgaccgccaaggtgtcccctggcctgtgcatcgccggggaccggggcatcgcccctaagagcggctacttcgtgaacgtgaacaacacctggatgttcaccggctccggctactactaccccgagcctatcaccggcaacaacgtggtggtcatgagcacctgcgccgtgaactacaccaaggcccccgacgtgatgctgaacatctccacccccaacctgcctgacttcaaggaggagctggaccagtggttcaagaaccagacctccgtggcccctgacctgagcctggactacatcaacgtgaccttcctggacctgcaggacgagatgaacaggctgcaggaggccatcaaggtgctgaaccagtcctacatcaacctgaaggacatcggcacctacgagtactacgtgaagtggcccagctccatcgccagcttcttcttcatcatcggcctgatcatcggcctgttcctggtgctgcgggtgggcatctacctgtgcatcaagctgaagcacaccaagaagcggcagatctacaccgacatcgagatgaacaggctgggcaagtaa

SEQ ID NO: 24 - DNA, 3351 bp, Artificial Sequence
  SADS-CoV Spike protein with VSV G protein TMD-CTD region
atgaagctgtttaccgtgtttaccctgctcgcctcaatcagagtgctctatggctgtgaatccgtggactttaacctgtttaataccatcttctccacccacaggggcctgtccaacaccaccagcgtgatcaccggcgcctaccccagcaccaacaagtccgactggagctgcaacaccaggaccggacacctgtccggaagcggattcggaatcggcctgtacgtgcagacccccagagagcagtaccagtacgacggatccggagctggaggatacaccatcgccgtgagcccaatccacgtgaccaacctgacctgggagctgtggatccacagaaagtggggcgtgaactccgtggtgaccgtgaggctgtgcagatggtggcagttcatgagcttcaactccaccagccacgctgctgacgctggacccaccaacgccttcgagtgcctgatcaacggcagctacccaacccaccgcaacaccggctacatgttcggcgtgacctggtacaacgacctggtgcggatcgtgttccccccaaccgtgctggagatgcagctggacggcctgcagtgggagagggtgcagttcaactccccagtgaacgctggacacgctacccggttcaacgtggtgaaggacatctccaccgtgctggtggagacaaactccggcggcagcgtgttcaggtacagctactgcgccgacggcttcgtgaacggcctgcagtgcaagctgagactgttcgacatccccccaggcgtgtactccaacagcgaggtggagtacccaaccgccctgtacaccgtggtgcacaacatgtccgcctgccccgagaggccagacagctactgcggctccaacagctgccccttcaagagagccgtgttcagcaactgcatcgtgaactacaccacctgggtgaacccagaccagcgcgacttccagcacctgatcctgcccaacggcaagttcaacccattcaccgagtgcaacggcctgaacaggatcgtggacggatgcgtgccaggattcgtgctgagggtgggaaggggcaaggccgtgaaccggaccatcgtgaccccctacctgaagccatacgagtgcttcggctggtcctggaacgacaaccaggacagcatctacgactggtggatcgccgacttcgtgtccaccggagccttcgtgtgcgagagcaacccagaggctccaaagaccggcgtgtgcgtgacctacaccgtggagaaggtgaccttccagggcgtgctgtacgagtccaacttcaccttcgcccagtactacaacctgctgtacgtgggcagccagctgagatacgtgagaatcctgggcaaggtgtacgaggtgtccagctgtttcgaggccagctacgacgtgctgtaccgcaacaaccagtccttcggcctgctgtaccggagcttcgactgcaaccagctgcacatcaagtccgccagattcgtggacaggctgctgccaagccacaacggaaccgccaccgtgctgggctgcctgttcaacgcctcctacgcccccaacgacaccatggtgaactgcaccaacccactgggcgacggattctgcgctgacctgctgggaaacgtggccgtgcgcaggatgaccttcgagaagcacgacaccacctacgtggcccccgtgaccaacgagaggtacaccgagatgccactggaccaccagctgatcctgaccgagcagttcctgcagaccaccatgccaaagttctccgtgagctgcgagacatacatctgcgacgtgagcaaggcctgcaagaacctgctgttcaggtacggcggcttctgccagaaggtggaggccgacatcagaggagctggcatcctgctggacggcgacgtgtccagcctgtactccaccatcgctgctaagacctccagcgtggtgccaaccaccgacaggttcaacgtgagccagttcttcctgccaaagacccagtccagcgccaacaagtacgagtccagaagcgccatcgaggacctgctgttctccaagatcgagacaaccggccccggcttctacggcgactactacaactgcaagaagaacgccatccaggacctgacctgcgcccagtaccacaacggcatcctggtcatccccccaatcatggacgccgagacactgggaatgtacggaggaatcgctgccgcctccgtgaccctgggaatcttcggaggacaggctggaatggctacctggagcgtggccatggccggccggctgaacgctctgggagtggtgcagaacgctctggtggacgacgtgaacaagctggccaacggcttcaaccagctgaccgcctccgtgagcaagctggccctgaccacctccagcgccctgcaggctatccaggctgtggtgaaccagaacgctgctcaggtggagtccctggtgagcggcatcaccgagaacttcggcgccatctccaccaacttcaaagtgatcagccagcgcctggacaagctggaggctgacgtgcagatggaccgcctgatcaacggccggatgaacgtgctgcagctgttcgtgaccaactacaagctgaagatcgccgagctgaggaacacccacagatacgtgcagtccctgatcaacgagtgcgtgtacgctcagagcctgaggaacggattctgcggacagggcctgcacgtgctgtccctgatgcagaacgcccccagcggcatcatgttcttccactacagcctgatccccaacaacaccatcaccgtgaagaccaccccaggcctgtgcgagtccgacgagctgggaagcaagtgcatcgtggctaaggacggcgtgctggtgtccgccaacctgagctactggcagtggtccccccgcaacctgtacaagccagagaacctgaccttcgccaacgtgatcgccgtgagcaggggagctaactacaccaccctgaacaagaccttcgacatccccgagctgaactccaccttcccaatcgaggaggagttccgcgagtacttccagaacatgtccagcgagctgcaggtgctgaagaacctgaccgccgacatgtccaagctgaacatcagcgccgagatccagctgatcaacgagatcgcccacaacgtgtccaacatgagggtggaggtggagaagttccagagatacgtgaactacgtgaagtcctccatcgcctccttcttcttcatcatcggcctgatcatcggcctgttcctggtgctgcgcgtgggcatctacctgtgcatcaagctgaagcacaccaagaagcgccagatctacaccgacatcgagatgaaccgcctgggcaag

(19) United States
(12) Patent Application Publication
(10) Pub. No.: US 2024/0000920 A1
(43) Pub. Date: Jan. 4, 2024

(54) RECOMBINANT VECTORS ENCODING CHIMERIC CORONAVIRUS SPIKE PROTEINS AND USE THEREOF

(71) Applicant: Intervet Inc., Madison, NJ (US)

(72) Inventors: Martijn Alexander Langereis, Boxmeer (NL); Ad De Groof, Boxmeer (NL); Paul Vermeij, Boxmeer (NL); Berend Jan Bosch, Utrecht (NL)

(73) Assignee: Intervet Inc., Madison, NJ (US)

(21) Appl. No.: 18/252,624

(22) PCT Filed: Nov. 11, 2021
(86) PCT No.: PCT/EP2021/081311

§ 371 Date: May 11, 2023

(30) Foreign Application Priority Data
Nov. 12, 2020 (EP) 20207302.9
Apr. 1, 2021 (EP) 21166533.6

(51) Int. Cl. A61K 39/215; C12N 15/86; C07K 14/005; A61P 31/14

(52) U.S. Cl. CPC A61K 39/215; C12N 15/86; C07K 14/005; A61P 31/14; C12N 2770/20022; C12N 2770/20034; C12N 2770/20071; C12N 2770/36143; C07K 2319/50; C07K 2319/35; C07K 2319/03; A61K 2039/5256

(57) ABSTRACT

The present invention provides recombinant vectors encoding a chimeric coronavirus spike protein. The present invention further provides new immunogenic compositions and vaccines comprising these recombinant vectors. Methods of administering these immunogenic compositions and vaccines to animal subjects, including humans, felines, and avians, to protect them against coronaviruses also are included. Methods of making the immunogenic compositions and vaccines alone or in combinations with other protective agents are provided too.

### FIELD OF THE INVENTION

The present invention relates to recombinant vectors encoding a chimeric coronavirus spike protein. The present invention further relates to new immunogenic compositions and vaccines comprising these recombinant vectors. The present invention further relates to methods of administering these immunogenic compositions and vaccines to animal subjects, including humans, to protect them against coronaviruses. In addition, the present invention relates to methods of making the immunogenic compositions and vaccines alone or in combinations with other protective agents.

### BACKGROUND OF THE INVENTION

Coronaviruses are enveloped, single stranded, non-segmented, positive sense RNA viruses that encode sixteen non-structural proteins, several accessory proteins, and four major structural proteins: (i) the spike surface protein (spike protein or S protein), which is a large glycoprotein protruding from the surface of the virus; (ii) an integral membrane (or matrix) protein (M); (iii) a small membrane envelope protein (E); and (iv) a nucleocapsid protein (N). The spike protein of a coronavirus determines the tropism of the coronavirus by binding to a specific extracellular domain of a host target protein that spans the membrane of the host cells of the infected animal. The target protein is denoted as the receptor.

All coronavirus S glycoproteins consist of four domains; the signal sequence, that is cleaved off during synthesis, the ectodomain which is present on the outside of the virion particle, the transmembrane region responsible for anchoring the S protein into the lipid bi-layer of the virion particle, and the cytoplasmic tail that might interact with other coronavirus proteins, such as the membrane protein (E) and the integral membrane protein (M). The coronavirus spike protein is a type I glycoprotein observable by electron microscopy as coronavirus virion spikes. The S protein is assembled into virion membranes, possibly through non-covalent interactions with the M protein, but is not required for formation of coronavirus virus-like particles. Following incorporation into coronavirus particles, determined by the carboxy-terminal domain, the S glycoprotein is responsible for binding to the target cell receptor and fusion of the viral and cellular membranes, fulfilling a major role in the infection of susceptible cells.

Coronaviruses are a large family of viruses that include avian coronaviruses, bovine coronaviruses, canine coronaviruses, feline coronaviruses, porcine coronaviruses, bat coronaviruses, and human coronaviruses. Infectious Bronchitis virus (IBV), an avian coronavirus, causes infectious bronchitis, which is an acute, highly contagious respiratory disease of domestic fowl (chicken). Clinical signs of Infectious Bronchitis include sneezing/snicking, tracheal rales, nasal discharge, and wheezing, and are more obvious in chicks than in adult birds. The birds also may appear depressed and consume less food. Meat-type birds have reduced weight-gain, whereas egg-laying birds lay fewer eggs. The respiratory infection predisposes chickens to secondary bacterial infections, which can be fatal in chicks. The virus can also cause permanent damage to the oviduct, especially in chicks, leading to reduced egg production and quality, and kidney, sometimes leading to kidney disease, which can be fatal.

The etiological agent of the worldwide human pandemic of 2019-2020, universally referred to as Coronavirus Disease 2019 (COVID-19), is a respiratory (and possibly also enteric) coronavirus named Severe Acute Respiratory Syndrome Coronavirus 2 (SARS-CoV-2). Although coronavirus infections in humans had been reported in the past century, they are generally associated with common cold-like symptoms, whereas SARS-CoV-2 follows the 2003 SARS epidemic (SARS-CoV) and the 2012 Middle East Respiratory Syndrome coronavirus (MERS-CoV) as the third major Betacoronavirus outbreak of the present millennium.

The host receptor for both SARS-CoV and SARS-CoV-2 is the angiotensin-converting enzyme 2 (ACE2), a type I integral membrane protein that is a zinc metalloenzyme that functions as a monocarboxypeptidase and plays an important role in vascular health. The primary function of ACE2 is to counterbalance the effect of the angiotensin-converting enzyme (ACE). ACE cleaves the angiotensin I hormone into the vasoconstricting peptide angiotensin II, whereas ACE2 cleaves the C-terminal amino acid of angiotensin II, ultimately resulting in the formation of a counter-acting vasodilating peptide. The binding of the spike protein of SARS-CoV-2 to ACE2 results in endocytosis and translocation of the virus into endosomes located within cells.

SARS-CoV-2 is thought to have zoonotic origins, with SARS-CoV-2 evolving from a bat coronavirus (bat CoV), either directly or through an intermediary animal [Wu et al., Cell Host & Microbe 27:1-4 (2020)]. Indeed, both SARS-CoV and SARS-CoV-2 are believed to have evolved from different SARS-like bat CoVs, that made their way into humans, potentially involving intermediary hosts. It has been suggested that SARS-CoV made its way to humans from bats via captive Himalayan palm civets (Paguma larvata) [Wu et al., supra; Guan et al., Science 302: 276-278 (2003)]. Notably, Himalayan palm civets also have been shown to be extremely susceptible to SARS-CoV [Kan et al., J. of Virol. 79(18):11892-11900 (2005); Guan et al., supra]. Consistently, comparing the nucleotide sequences of their entire genomes indicates that SARS-CoV-2 is genetically more closely related to SARS-like bat CoVs than to SARS-CoV [Wu et al., supra]

Like with SARS-CoV, there have been a number of reports in the general media of lions and tigers in zoos, and domestic cats, testing positive for SARS-CoV-2. Some of these felines, including domestic cats, have demonstrated clinical signs of infection and significant post-mortem lung lesions. Recent reports also have shown that SARS-CoV-2 can infect ferrets, hamsters, and mink.

Although, to date, there has been no report of humans contracting COVID-19 from domestic cats, it remains a great fear that such a transmission could occur. One basis for this fear comes from studies that report that infected domestic cats may shed sufficient SARS-CoV-2 by aerosol to infect other cats that have been kept physically distant. Furthermore, based on their rate of seroconversion, studies suggest that cat to cat transmission of SARS-CoV-2 may occur in a natural setting. Moreover, recent studies have shown that infected minks have transmitted SARS-CoV-2 to humans [Oreshkova et al., Eurosurveillance 25(23) (2020):pii=2001005; doi: 10.2807/1560-7917.ES.2020.25.23.2001005].

Somewhat encouraging, in preliminary studies SARS-CoV-2 has shown relatively little genetic diversity, suggesting that the right feline vaccine against SARS-CoV-2 may be successful. Ideally, such a vaccine would prevent transmission of the virus to cats, prevent cats from becoming a reservoir for the virus, and/or reduce the shedding of SARS-CoV-2 by infected cats. Currently, there are over 200 potential SARS-CoV-2 vaccines being developed for humans, with researchers employing many different vaccine strategies. However, even with this unprecedented effort, there remains uncertainty whether any one of these vaccine strategies will lead to a vaccine that will make a significant step in countering the spread of SARS-CoV-2 or the effect of COVID-19.

Even before the recent excitement over SARS-CoV-2, modifications of the human coronavirus spike protein to increase its immunogenicity and/or availability to the host immune system already had become of some interest. Relying on prior findings with the fusion proteins from HIV-1 and respiratory syncytial virus (RSV), that showed that proline substitutions in the loop between the first heptad repeat (HR1) and the central helix restricted premature triggering of the fusion protein and resulting in greater than a 50-fold improvement in ectoderm yield, due to the introduction of two consecutive proline substitutions (referred to as 2P) at residues V1060 and L1061, it was demonstrated that homologous substitutions in the spike proteins from SARS-CoV and HCoV-HKU1 also increased the expression levels of the ectodomains and improved conformational homogeneity [Pallesen et al., Proceedings of the National Academy of Sciences of the United States of America 114: E7348-e7357, https://doi.org/10.1073/pnas.1707304114 (2017)]. Moreover, the introduction of two consecutive proline residues (2P) at the beginning of the central helix was suggested to be a general strategy for retaining Betacoronavirus S proteins in the prototypical prefusion conformation [Pallesen et al., supra]. More recently, Amanat et al., [doi: https://doi.org/10.1101/2020.09.16.300970, (2020)] have reported that the introduction of two prolines and removal of the polybasic cleavage site leads to optimal efficacy of a recombinant spike based SARS-CoV-2 vaccine in the mouse model. Other modifications of the human coronavirus spike proteins have also been discussed [see, Sternberg and Naujokat, Life Sciences, 257:118056 (2020); Li, Ann. Rev. Viol., 3(1): 237-261 (2016); and Wickramasinghe et al., Virus Research 194: 37-48 (2014)].

The causative agent of a fatal swine acute diarrhoea syndrome (SADS) in pigs is a novel coronavirus that is 98.48% identical in genome sequence to a bat coronavirus, HKU2. The HKU2-related coronavirus was detected in 2016 in bats in a cave in the vicinity of a pig farm. This new coronavirus virus, swine acute diarrhoea syndrome coronavirus (SADS-CoV), originated from the same genus of horseshoe bats (Rhinolophus) as SARS-CoV [Zhou et al., Nature, 556: 255-258 (2018); doi.org/10.1038/s41586-018-0010-9].

Vesicular stomatitis virus (VSV) is a non-segmented negative-strand RNA virus that is in the Rhabdoviridae family, which includes rabies virus. VSV buds preferentially from the basolateral surface of polarized epithelial cells. This budding preference correlates with the basolateral localization of its glycoprotein [see, e.g., Drokhlyansky et al., J. Virol., 89(22): 11718-11722 (2015)]. Such plasma membrane budding enables viruses to exit the host cell and is mostly used by enveloped viruses which must acquire a host-derived membrane enriched in viral proteins to form their external envelope. Nucleocapsids assembled or in the process of being built induce formation of a membrane curvature in the host cell membrane and wrap up in the forming bud, which is eventually pinched off by membrane scission to release the enveloped particle.

The use of alphavirus-derived RNA replicon particles (RPs) is one of the large number of vector strategies that have been employed in vaccines through the years to protect against specific animal pathogens [Vander Veen, et al. Anim Health Res Rev. 13(1):1-9. (2012) doi: 10.1017/S1466252312000011; Kamrud et al., J Gen Virol. 91 (Pt 7):1723-1727 (2010)]. Alphavirus-derived RPs have been developed for several different alphaviruses, including Venezuelan equine encephalitis virus (VEEV) [Pushko et al., Virology 239:389-401 (1997)], Sindbis (SIN) [Bredenbeek et al., Journal of Virology 67:6439-6446 (1993)], and Semliki Forest virus (SFV) [Liljestrom and Garoff, Biotechnology (NY) 9:1356-1361 (1991)]. Alphavirus RP vaccines deliver propagation-defective alphavirus RNA replicons into host cells and result in the expression of the desired immunogenic transgene(s) in vivo [Pushko et al., supra]. The construction of a hybrid VEEV/SIN replication particle encoding the SARS-CoV spike protein that expresses detectable spike protein, in vitro, has been reported [U.S. Pat. No. 9,730,997]. RPs also have an attractive safety and efficacy profile when compared to some traditional vaccine formulations [Vander Veen, et al. Anim Health Res Rev. 13(1):1-9 (2012)]. Furthermore, the VEEV RP platform has been used to encode pathogenic antigens from canines and felines [see e.g., WO2019/086645, WO2019/086646, and WO2019/115090] and is the basis for several USDA-licensed vaccines for swine and poultry.

The citation of any reference herein should not be construed as an admission that such reference is available as “prior art” to the instant application.

### SUMMARY OF THE INVENTION

Accordingly, the present invention provides recombinant vectors that encode modified coronavirus spike proteins. In particular embodiments, the modified coronavirus spike protein is a chimeric coronavirus spike protein. The vectors that encode the modified coronavirus spike proteins (e.g., a chimeric coronavirus spike protein) can be used in immunogenic compositions and/or in vaccines. In specific embodiments, the recombinant vector is a recombinant expression vector. In other embodiments, the recombinant vector is a synthetic messenger RNA (synthetic mRNA).

One aspect of the present invention provides a recombinant vector encoding a chimeric coronavirus spike protein that comprises a spike protein originating from a coronavirus, and a transmembrane domain (TMD) and a C-terminal domain (CTD) from a surface glycoprotein originating from a budding virus that buds from a host cell's plasma membrane (BVₚₘ), in place of a TMD and a CTD of the coronavirus spike protein. In particular embodiments of this type, the recombinant vector is a recombinant BVₚₘ, and the TMD and CTD of the surface glycoprotein originates from a virus species that is different from that of the recombinant BVₚₘ.

In certain embodiments of the recombinant vector, the surface glycoprotein that originates from a BVₚₘ is a glycoprotein (G protein) from a VSV. In other embodiments of the recombinant vector, the surface glycoprotein that originates from a BVₚₘ is a hemagglutinin of an influenza virus. In yet other embodiments of the recombinant vector, the surface glycoprotein that originates from a BVₚₘ is a neuraminidase of an influenza virus. In still other embodiments of the recombinant vector, the surface glycoprotein that originates from a BVₚₘ is a hemagglutinin-neuraminidase (HN) protein of a Newcastle Disease virus (NDV). In yet other embodiments of the recombinant vector, the surface glycoprotein that originates from a BVₚₘ is a fusion (F) protein of a NDV. In still other embodiments of the recombinant vector, the surface glycoprotein that originates from a BVₚₘ is a glycoprotein 120 (gp120) of a human immunodeficiency virus (HIV). In yet other embodiments of the recombinant vector, the surface glycoprotein that originates from a BVₚₘ is a glycoprotein (GP) of a Lassa virus. In still other embodiments of the recombinant vector, the surface glycoprotein that originates from a BVₚₘ is a GP of an Ebola virus. In yet other embodiments of the recombinant vector, the surface glycoprotein that originates from a BVₚₘ is a F protein of a Measles virus (MV).

In still other embodiments of the recombinant vector, the surface glycoprotein that originates from a BVₚₘ is a HN protein of a MV.

In a related aspect, the present invention provides recombinant vectors that encode a chimeric coronavirus spike protein in which the furin cleavage site of the chimeric coronavirus spike protein is inactivated. In other embodiments, the recombinant vectors that encode a chimeric coronavirus spike protein that is further stabilized in a prefusion state due to the replacement of two consecutive amino acid residues at the beginning of the central helix of the chimeric coronavirus spike protein by a pair of proline residues (2P).

In related embodiments, the recombinant vectors encode a chimeric coronavirus spike protein in which both the furin cleavage site of the chimeric coronavirus spike protein is inactivated and the chimeric coronavirus spike protein is further stabilized in a prefusion state due to the replacement of two consecutive amino acid residues at the beginning of the central helix of the coronavirus spike protein by a pair of proline residues (2P).

In particular embodiments, the recombinant vectors comprise a chimeric coronavirus spike protein, in which the coronavirus spike protein portion of the chimeric coronavirus spike protein originates from a mammalian coronavirus. In certain embodiments of the recombinant vectors, the coronavirus spike protein portion of the chimeric coronavirus spike protein originates from a bovine coronavirus. In still other embodiments of the recombinant vectors, the coronavirus spike protein portion of the chimeric coronavirus spike protein originates from a canine coronavirus. In yet other embodiments of the recombinant vectors, the coronavirus spike protein portion of the chimeric coronavirus spike protein originates from a feline coronavirus. In still other embodiments of the recombinant vectors, the coronavirus spike protein portion of the chimeric coronavirus spike protein originates from a porcine coronavirus. In particular embodiments of the recombinant vectors of this type, the porcine coronavirus is a SADS-CoV. In other particular embodiments of the recombinant vectors of this type, the porcine coronavirus is a porcine epidemic diarrhoea virus (PEDV). In yet other embodiments of the recombinant vectors, the coronavirus spike protein portion of the chimeric coronavirus spike protein originates from a bat coronavirus.

In more particular embodiments of the recombinant vectors, the coronavirus spike protein portion of the chimeric coronavirus spike protein originates from a human coronavirus. In specific embodiments of the recombinant vectors of this type, the coronavirus spike protein portion of the chimeric coronavirus spike protein originates from a SARS-CoV. In still other embodiments of the recombinant vectors of this type, the coronavirus spike protein portion of the chimeric coronavirus spike protein originates from MERS. In even more particular embodiments of the recombinant vectors of this type, the coronavirus spike protein portion of the chimeric coronavirus spike protein originates from SARS-CoV-2.

In specific embodiments of the recombinant vectors, the chimeric coronavirus spike protein comprises 80%, 85%, 90%, 95%, 97%, 99%, 99.5%, or greater identity with amino acid residues 14 to 1211 of the amino acid sequence of SEQ ID NO: 10, over the same range of amino acid residues, and the chimeric coronavirus spike protein comprises an inactivated furin cleavage site. In more specific embodiments of this type of the recombinant vectors, the chimeric coronavirus spike protein further comprises 80%, 85%, 90%, 95%, 97%, or greater identity with amino acid residues 1212 to 1260 of the amino acid sequence of SEQ ID NO: 10, over the same range of amino acid residues. In even more specific embodiments of the recombinant vectors, the chimeric coronavirus spike protein comprises the amino acid sequence of SEQ ID NO: 10.

In other specific embodiments of the recombinant vectors, the chimeric coronavirus spike protein comprises 80%, 85%, 90%, 95%, 97%, 99%, 99.5%, or greater identity with amino acid residues 14 to 1211 of the amino acid sequence of SEQ ID NO: 12, over the same range of amino acid residues, and the chimeric coronavirus spike protein comprises both an inactivated furin cleavage site, and the lysine (K) residue at position 986 and the valine (V) residue at position 987 of SEQ ID NO: 12 are replaced by a pair of proline residues (2P). In more specific embodiments of this type of the recombinant vectors, the chimeric coronavirus spike protein further comprises 80%, 85%, 90%, 95%, 97%, or greater identity with amino acid residues 1212 to 1260 of the amino acid sequence of SEQ ID NO: 12, over the same range of amino acid residues. In even more specific embodiments of the recombinant vectors, the chimeric coronavirus spike protein comprises the amino acid sequence of SEQ ID NO: 12.

In still other embodiments of the recombinant vectors, the coronavirus spike protein portion of the chimeric coronavirus spike protein originates from an avian coronavirus. In particular embodiments of the recombinant vectors of this type, the avian coronavirus is an IBV. In more specific embodiments of the recombinant vectors, the IBV is a Massachusetts serotype. In even more particular embodiments of this type of recombinant vectors, the coronavirus spike protein portion of the chimeric coronavirus spike protein originates from an IBV-Ma5. In other embodiments of the recombinant vectors, the coronavirus spike protein portion of the chimeric coronavirus spike protein originates from a serotype 4/91 IBV. In yet other related embodiments of the recombinant vectors, the coronavirus spike protein portion of the chimeric coronavirus spike protein originates from a serotype QX IBV.

In specific embodiments of these recombinant vectors, the chimeric coronavirus spike protein comprises 80%, 85%, 90%, 95%, 97%, 99%, 99.5%, or greater identity with amino acid residues 19 to 1091 of the amino acid sequence of SEQ ID NO: 4, over the same range of amino acid residues, and the chimeric coronavirus spike protein comprises an inactivated furin cleavage site. In more specific embodiments of this type of the recombinant vectors, the chimeric coronavirus spike protein further comprises 80%, 85%, 90%, 95%, 97%, or greater identity with amino acid residues 1092 to 1140 of the amino acid sequence of SEQ ID NO: 4, over the same range of amino acid residues. In even more specific embodiments of the recombinant vectors, the chimeric coronavirus spike protein comprises the amino acid sequence of SEQ ID NO: 4.

In other specific embodiments of the recombinant vectors, the chimeric coronavirus spike protein comprises 80%, 85%, 90%, 95%, 97%, 99%, 99.5%, or greater identity with amino acid residues 19 to 1091 of the amino acid sequence of SEQ ID NO: 6, over the same range of amino acid residues, and the chimeric coronavirus spike protein comprises both an inactivated furin cleavage site, and the alanine (A) residue at position 859 and the isoleucine (I) residue at position 860 of SEQ ID NO: 6 are replaced by a pair of proline residues (2P). In more specific embodiments of this type of the recombinant vectors, the chimeric coronavirus spike protein further comprises 80%, 85%, 90%, 95%, 97%, or greater identity with amino acid residues 1092 to 1140 of the amino acid sequence of SEQ ID NO: 6, over the same range of amino acid residues. In even more specific embodiments of the recombinant vectors, the chimeric coronavirus spike protein comprises the amino acid sequence of SEQ ID NO: 6.

In one aspect of the present invention, the recombinant vector of the present invention is a recombinant expression vector. In particular embodiments of this type, the recombinant expression vector is a recombinant viral vector. In other embodiments, the recombinant expression vector is a DNA expression plasmid.

In particular embodiments, the recombinant viral vector is a recombinant avian viral vector. In more particular embodiments this type, the recombinant viral vector is a recombinant herpesvirus of turkeys (HVT). In yet other embodiments, the recombinant viral vector is a recombinant attenuated Marek's disease virus 1 (MDV1). In still other embodiments, the recombinant viral vector is a recombinant attenuated Marek's disease virus 2 (MDV2). In yet other embodiments, the recombinant viral vector is a recombinant attenuated NDV.

In other embodiments, the recombinant viral vector is a recombinant attenuated MV. In still other embodiments, the recombinant viral vector is an alphavirus RNA replicon particle (RP). In specific embodiments of this type, the alphavirus RNA replicon particle is a VEEV RNA replicon particle.

In even more particular embodiments, the alphavirus RNA RPs comprises the capsid protein and glycoproteins of the avirulent TC-83 strain of VEEV.

In specific embodiments, the recombinant viral vector is a VEEV RNA replicon particle that encodes a chimeric coronavirus spike protein of the present invention. In more specific embodiments, the chimeric coronavirus spike protein is a SARS-CoV-2-VSV spike protein. In other specific embodiments, the recombinant viral vector is a recombinant HVT vector that encodes a chimeric coronavirus spike protein of the present invention. In more specific embodiments, the chimeric coronavirus spike protein is a chimeric IBV-VSV spike protein of the present invention.

In still other embodiments, the recombinant expression vector is a DNA expression plasmid. In particular embodiments of this type, the DNA expression plasmid encodes an RNA replicon. In even more particular embodiments, the RNA replicon is a VEEV RNA replicon.

In yet other embodiments the recombinant vector is a synthetic mRNA.

In particular embodiments, the recombinant vectors further encode one or more other antigens. In certain embodiments of this type, the recombinant vectors comprise a chimeric coronavirus spike protein and further encode a second coronavirus antigen. In specific embodiments of this type, the chimeric coronavirus spike protein is a chimeric SARS-CoV-2 spike protein and the second coronavirus antigen is a second SARS-CoV-2 protein antigen. In more particular embodiments, the second SARS-CoV-2 protein antigen is an integral membrane (or matrix) protein (M). In other embodiments the second SARS-CoV-2 protein antigen is a small membrane envelope protein (E). In still other embodiments, the second SARS-CoV-2 protein antigen is a nucleocapsid protein (N). In more particular embodiments, the second SARS-CoV-2 protein antigen is a second chimeric SARS-CoV-2 spike protein in which the spike protein portion of the two chimeric SARS-CoV-2 spike protein originate from different strains of SARS-CoV-2.

In other embodiments, the recombinant vectors encode a first chimeric SARS-CoV-2 spike protein, optionally together with the second chimeric SARS-CoV-2 spike protein and/or a second SARS-CoV-2 antigen, and an antigen from a non-SARS-CoV-2. In certain embodiments, the non-SARS-CoV-2 antigen is a feline calicivirus (FCV) capsid protein. In yet other embodiments the non-SARS-CoV-2 antigen is a rabies virus glycoprotein (G). Still other embodiments, the non-SARS-CoV-2 antigen is feline leukemia virus (FeLV) envelope protein. In yet other embodiments, the non-SARS-CoV-2 antigen is a human influenza virus protein. In particular embodiments of this type, the human influenza virus protein is a hemagglutinin. In another embodiment, the human influenza virus protein is a neuraminidase.

The present invention further provides immunogenic compositions comprising one or more of the recombinant vectors of the present invention. In particular embodiments, the immunogenic compositions comprise a pharmaceutically acceptable carrier. The recombinant vectors can be a recombinant expression vector, e.g., recombinant viral vectors and DNA expression plasmids; or a synthetic mRNA. The present invention further provides vaccines that comprise one or more of the immunogenic compositions and a pharmaceutically acceptable carrier.

Accordingly, an immunogenic composition and/or vaccine of the present invention can comprise one or more of any of the recombinant vectors of the present invention, including any recombinant viral vectors, any DNA expression plasmid of the present invention and/or any synthetic mRNA of the present invention. In certain embodiments the immunogenic composition and/or vaccine further comprises a pharmaceutically acceptable carrier.

In certain embodiments, vaccines to aid in the protection of a mammal from an infection by SARS-CoV-2 comprise a recombinant vector encoding a chimeric SARS-CoV-2 spike protein that comprises a spike protein originating from SARS-CoV-2, and a TMD and a CTD from a surface glycoprotein originating from a budding virus that buds from a host cell's plasma membrane (BVₚₘ), in place of a TMD and a CTD of the SARS-CoV-2 spike protein. In particular embodiments of this type, the recombinant vector is a recombinant BVₚₘ, and the TMD and CTD of the surface glycoprotein originates from a virus species that is different from that of the recombinant BVₚₘ. In more specific embodiments, the surface glycoprotein of the BVₚₘ is the G protein of a vesicular stomatitis virus. In certain embodiments of this type, the mammal is a human. In other embodiments, the vaccines are to aid in reducing shedding of SARS-CoV-2 in a feline or a ferret due to an infection of SARS-CoV-2 in the feline or the ferret.

Accordingly, in certain embodiments, a vaccine of the present invention induces sterile immunity in a vaccinated mammal. In still other embodiments, a vaccine of the present invention prevents the transmission of a coronavirus from a vaccinated mammal to a naïve mammal. In related embodiments, a vaccine of the present invention both induces sterile immunity in a vaccinated mammal and prevents the transmission of coronavirus from the vaccinated mammal to a naïve mammal. In particular embodiments of this type, the vaccinated mammal is a feline. In more particular embodiments of this type, the vaccinated mammal is a cat (e.g., domestic cat). In yet other embodiments, the naïve mammal is a feline. In more particular embodiments of this type, the feline is a cat (e.g., domestic cat). In related embodiments, both the vaccinated mammal and the naïve mammal are cats (e.g., domestic cats). In certain embodiments, such mammalian (e.g., feline) vaccines comprise an adjuvant. In other such embodiments, the mammalian (e.g., feline) vaccine is a non-adjuvanted vaccine.

In alternative embodiments, a vaccine is to aid in the protection of infectious bronchitis in an avian due to an infection of IBV in the avian, comprising a recombinant vector encoding a chimeric IBV spike protein that comprises a spike protein originating from an IBV, and a TMD and a CTD from a surface glycoprotein originating from a budding virus that buds from a host cell's plasma membrane (BVₚₘ), in place of a TMD and a CTD of the coronavirus spike protein. In particular embodiments of this type, the recombinant vector is a recombinant BVₚₘ, and the TMD and CTD of the surface glycoprotein originates from a virus species that is different from that of the recombinant BVₚₘ. In more specific embodiments, the surface glycoprotein of the BVₚₘ is the G protein of a vesicular stomatitis virus. In related embodiments, the vaccines are to aid in the protection of a chicken from infectious bronchitis due to an infection of IBV in the chicken.

The present invention further provides methods of immunizing a mammal against a coronavirus, e.g., SARS-CoV-2, comprising administering to the mammal an immunologically effective amount of a vaccine of the present invention. In certain embodiments, the method of administering is performed by intramuscular administration (IM). In other embodiments, the method of administering is performed by subcutaneous administration (SC). In still other embodiments, the method of administering is performed by intradermal administration (ID). In yet other embodiments, the method of administering is performed by oral administration. In still other embodiments, the method of administering is performed by intranasal administration. The vaccines of the present invention can be administered either as a one dose administration (e.g., as a single-dose vaccine) or with one or more subsequent booster administrations.

In particular embodiments, the mammal is a feline. In more particular embodiments, the feline is a domestic cat. In still other embodiments, the feline is a lion. In yet other embodiments, the feline is a tiger. In related embodiments, the mammal is a ferret. In alternative embodiments, the mammal is a human.

The present invention further provides methods of inducing sterile immunity against a coronavirus in a mammal comprising administering an effective amount of one of the vaccines of the present invention to the mammal, thus providing a mammalian vaccine. In still other embodiments, the present invention provides methods of preventing the transmission of coronavirus from a vaccinated mammal to a naïve mammal comprising administering an effective amount of one of the mammalian vaccines of the present invention to the mammal. In related embodiments, the present invention provides methods of both inducing sterile immunity against a coronavirus in a mammal and preventing the transmission of coronavirus from a vaccinated mammal to a naïve mammal comprising administering an effective amount of one of the mammalian vaccines of the present invention to the mammal. In particular embodiments of this type, the vaccinated mammal is a feline. In more particular embodiments of this type, the mammal vaccinated is a cat (e.g., domestic cat). In yet other embodiments, the naïve mammal is a feline. In more particular embodiments of this type, the feline is a cat (e.g., domestic cat). In related embodiments, both the mammal vaccinated and the naïve mammal are cats (e.g., domestic cats). In certain embodiments, such mammalian (e.g., feline) vaccines comprise an adjuvant. In other such embodiments, the mammalian (e.g., feline) vaccine is a non-adjuvanted vaccine.

The present invention further provides methods of immunizing an avian against IBV comprising administering to the avian an immunologically effective amount of a vaccine of the present invention. Accordingly, the vaccines of the present invention can be administered to the avian by parenteral administration. In particular embodiments, the vaccine is administered to the avian by intramuscular administration (IM). In still other embodiments, the vaccine is administered to the avian by subcutaneous administration (SC). In yet other embodiments, the vaccine is administered to the avian by intradermal administration (ID). In still other embodiments, the vaccine is administered to the avian by oral administration. In yet other embodiments, the vaccine is administered to the avian by intranasal administration. In still other embodiments, the vaccine is administered to the avian by in ovo administration. In still other embodiments, the vaccine is administered to the avian by scarification. In more specific embodiments, the avian is a chicken. The vaccines of the present invention can be administered either as a one dose administration (e.g., as a single-dose vaccine) or with one or more subsequent booster administrations.

Immunogenic compositions and/or vaccines (including multivalent vaccines) comprising a recombinant vector of the present invention, e.g., an alphavirus RNA replicon particle encoding a chimeric SARS-CoV-2 spike protein or a recombinant HVT encoding a chimeric IBV spike protein, can be administered in the presence, or alternatively, in the absence of an adjuvant.

In particular embodiments, the adjuvant is an oil adjuvant comprising more than one oil, e.g., a mineral oil and one or more non-mineral oils. In certain embodiments of this type the oil adjuvant comprises a liquid paraffin oil as the mineral oil, and one or more non-mineral oils selected from squalane, squalene, vitamin E, vitamin E-acetate, oleate, and ethyl-oleate. In more particular embodiments, the oil adjuvant comprises a liquid paraffin oil and vitamin E-acetate. In alternative embodiments, the vaccines do not comprise an adjuvant and are non-adjuvanted vaccines.

In yet another aspect, the present invention provides chimeric coronavirus spike proteins that comprise a spike protein originating from a SARS-CoV-2, and a TMD and a CTD of a surface glycoprotein originating from a vesicular stomatitis virus, in place of a TMD and a CTD of the SARS-CoV-2 spike protein. In specific embodiments, the chimeric coronavirus spike proteins comprises 80%, 85%, 90%, 95%, 97%, 99%, 99.5%, or greater identity with amino acid residues 14 to 1211 of the amino acid sequence of SEQ ID NO: 10, over the same range of amino acid residues, and the chimeric coronavirus spike protein comprises an inactivated furin cleavage site. In more specific embodiments of this type, the chimeric coronavirus spike protein further comprises 80%, 85%, 90%, 95%, 97%, or greater identity with amino acid residues 1212 to 1260 of the amino acid sequence of SEQ ID NO: 10, over the same range of amino acid residues. In even more specific embodiments, the chimeric coronavirus spike protein comprises the amino acid sequence of SEQ ID NO: 10.

In specific embodiments, the chimeric coronavirus spike protein comprises 80%, 85%, 90%, 95%, 97%, 99%, 99.5%, or greater identity with amino acid residues 14 to 1211 of the amino acid sequence of SEQ ID NO: 12, over the same range of amino acid residues, and the chimeric coronavirus spike protein comprises both an inactivated furin cleavage site, and the lysine (K) residue at position 986 and the valine (V) residue at position 987 of SEQ ID NO: 12 are replaced by a pair of proline residues (2P). In certain embodiments of this type, the chimeric coronavirus spike protein further comprises 80%, 85%, 90%, 95%, 97%, or greater identity with amino acid residues 1212 to 1260 of the amino acid sequence of SEQ ID NO: 12, over the same range of amino acid residues. In even more specific embodiments, the chimeric coronavirus spike protein comprises the amino acid sequence of SEQ ID NO: 12.

The present invention further provides nucleic acids that encode one or more of the chimeric coronavirus spike proteins that comprise a spike protein originating from a SARS-CoV-2, and a TMD and a CTD of a surface glycoprotein originating from a vesicular stomatitis virus, in place of a TMD and a CTD of the SARS-CoV-2 spike protein.

In still another aspect, the present invention provides chimeric coronavirus spike proteins that comprise a spike protein originating from an IBV, and a TMD and a CTD of a surface glycoprotein originating from a vesicular stomatitis virus, in place of a TMD and a CTD of the IBV spike protein. In specific embodiments, the chimeric coronavirus spike protein comprises 80%, 85%, 90%, 95%, 97%, 99%, 99.5%, or greater identity with amino acid residues 19 to 1091 of the amino acid sequence of SEQ ID NO: 4, over the same range of amino acid residues, and the chimeric coronavirus spike protein comprises an inactivated furin cleavage site. In more specific embodiments, the chimeric coronavirus spike protein further comprises 80%, 85%, 90%, 95%, 97%, or greater identity with amino acid residues 1092 to 1140 of the amino acid sequence of SEQ ID NO: 4, over the same range of amino acid residues. In even more specific embodiments, the chimeric coronavirus spike protein comprises the amino acid sequence of SEQ ID NO: 4.

In specific embodiments, the chimeric coronavirus spike protein comprises 80%, 85%, 90%, 95%, 97%, 99%, 99.5%, or greater identity with amino acid residues 19 to 1091 of the amino acid sequence of SEQ ID NO: 6, over the same range of amino acid residues, and the chimeric coronavirus spike protein comprises both an inactivated furin cleavage site, and the alanine (A) residue at position 859 and the isoleucine (I) residue at position 860 of SEQ ID NO: 6 are replaced by a pair of proline residues (2P). In certain embodiments of this type, the chimeric coronavirus spike protein further comprises 80%, 85%, 90%, 95%, 97%, or greater identity with amino acid residues 1092 to 1140 of the amino acid sequence of SEQ ID NO: 6, over the same range of amino acid residues. In even more specific embodiments, the chimeric coronavirus spike protein comprises the amino acid sequence of SEQ ID NO: 6.

The present invention further provides nucleic acids that encode one or more of the chimeric coronavirus spike proteins that comprise a spike protein originating from an IBV, and a TMD and a CTD of a surface glycoprotein originating from a vesicular stomatitis virus, in place of a TMD and a CTD of the IBV spike protein.

These and other aspects of the present invention will be better appreciated by reference to the following Brief Description of the Drawings and the Detailed Description.

### BRIEF DESCRIPTION OF THE DRAWINGS

FIG. 1 shows the results from a commercial ID Screen® Infectious Bronchitis Indirect (IDVet) test.

FIG. 2 shows the results from ciliostasis assays with recombinant viral constructs that encode modified IBV spike proteins.

FIG. 3 shows the results of the SARS-CoV-2 RBD Surrogate Pseudo-VN test.

FIG. 4 shows the results of the SARS-CoV-2 RBD Surrogate Pseudo-VN test after a boost vaccination 3 weeks after the initial vaccination.

FIGS. 5A-5F show the immunogenicity study of vaccine candidates in a guinea pig model.

FIG. 5A provides the overview of animal handlings: V=vaccination and B=blood sampling.

FIG. 5B shows surrogate SARS-CoV-2 virus neutralization (VN) tests performed using 10-fold diluted serum samples from day 21 (D21).

FIG. 5C shows surrogate SARS-CoV-2 VN tests performed using 1,000-fold diluted serum samples from day 35, 49 and 63/64 post prime vaccination (d.p.v.). The black line with circles shows the antibody levels induced by the Spike-wt antigen and the gray line with squares shows the antibody levels induced by the Spike-FCS-2P-VSV antigen.

FIG. 5D shows the indirect ELISA results using the SARS-CoV-2 Spike RBD (left) or ectodomain (right) as the antigen. Shown are EC50 values of sera (expressed as fold dilution) from cats exposed to the Spike-wt antigen (black line with circles) or the Spike-FCS-2P-VSV antigen (gray line with squares).

FIG. 5E provides the results of the lymphocyte stimulation test (LST) from blood collected on day 70/71. Purified SARS-CoV-2 S1 antigen was used to stimulate isolated lymphocytes and proliferation was measured 96 hours after stimulation.

FIG. 5F provides the surrogate VN test performed using 2-fold diluted swab samples taken at day 70/71.

FIGS. 6A-6E depicts a vaccination-challenge experiment in cats.

FIG. 6A provides an overview of animal handlings: V=vaccination, B=blood sampling, O=oropharyngeal swabs, N=nasal wash, (all)=all animals, (ch)=only challenged animals, (sen)=only sentinel animals.

FIG. 6B shows the serum neutralizing antibody titers determined using a SARS-CoV-2 VN test 21- and 45-days post vaccination (d.p.v.). The black line with open squares shows the antibody levels in the control-vaccinated animals, the black line with black triangles shows the antibody levels in non-vaccinated sentinel animal, and the gray line with closed squares show antibody titers induced by the Spike-FCS-2P-VSV antigen.

FIG. 6C shows serum neutralizing antibody titers determined using a SARS-CoV-2 VN test at day of challenge, 45-days post vaccination (open squares) and 12 (challenged) or 14 (sentinel) days post challenge (closed squares).

FIG. 6D shows SARS-CoV-2 virus titers in pfu/ml in oropharyngeal swabs 1 till 8 days post challenge (d.p.c.). The black line with open squares shows viral titers in challenged control-vaccinated animals, the black line with triangles shows viral titers in non-vaccinated sentinel animals co-housed with control-vaccinated animals, the gray line with closed squares shows viral titers in Spike-FCS-2P-VSV antigen vaccinated animals, and the black line with downwards pointing triangles shows viral titers in non-vaccinated sentinel animals co-housed with Spike-FCS-2P-VSV antigen vaccinated animals.

FIG. 6E shows SARS-CoV-2 virus titers in plaque forming units (pfu)/ml in nasal wash after challenge. The lines and symbols of FIG. 6E are the same as in FIG. 6D.

FIG. 7 provides a schematic representation of the wildtype SARS-CoV-2 Spike antigen (Spike-wt) and the stabilized SARS-CoV-2 Spike antigen (Spike-FCS-2P-VSV). The different Spike protein domains are indicated by different grey shadings. Also, the furin cleavage site mutation (AFCS, R682A/R683A), 2P substitutions (K986P/V987P) and TM-CTD replacements are depicted.

FIGS. 8 and 9 describe the effects on total- and on surface expression levels of chimeric spike proteins from BCoV respectively from SADS-CoV, as tested by FACS on Vero host cells. Details are given in Example 11.

### DETAILED DESCRIPTION OF THE INVENTION

The present invention provides immunogenic compositions and vaccines that aid in the prevention of, or even in some cases prevent disease, in mammals such as humans, felines, and ferrets, in avians such as chickens, porcine, bovine, and canines, caused by coronaviruses. Moreover, as shown in the Example 10 below, the present invention further provides immunogenic compositions and vaccines that induce sterile immunity in a mammal.

In one aspect of the invention, the coronavirus is SARS-CoV-2, and the disease is in humans, cats, and/or ferrets. These vaccines may not only be beneficial to the vaccinated humans, cats, and/or ferrets, but particularly in the case of the cats and ferrets, also may prevent them from becoming a reservoir for the virus, where further unknown and potentially deleterious mutations could arise.

Moreover, such vaccines could lead to the reduction or even elimination of the viral shed of SARS-CoV-2 in the cats and/or ferrets. Such viral shed could result in the transmission of SARS-CoV-2 to other animals, including humans. Accordingly, the present invention further provides immunogenic compositions and vaccines that prevent transmission of coronavirus from infected animals to naïve animals.

In another aspect of the invention, the coronavirus is IBV, and the disease is in poultry such as chickens. In another aspect of the invention, the coronavirus is IBV, and the disease is in swine. In a particular embodiment of this type, the coronavirus is SADS-CoV. In a particular embodiment of this type, the coronavirus is PEDV and the disease is in swine.

Accordingly, the present invention provides immunogenic compositions and/or vaccines (including multivalent vaccines) that comprise a recombinant vector that encodes a chimeric coronavirus spike protein. In certain embodiments the chimeric coronavirus spike protein comprises: a receptor binding domain (RBD) of a coronavirus spike protein, a furin cleavage site of the coronavirus spike protein, and a central helix of the coronavirus spike protein, but a TMD and a CTD of a surface glycoprotein of a budding virus that buds from a host cell's plasma membrane (BVₚₘ), e.g. in which the TMD and the CTD of the coronavirus spike protein is replaced by the TMD and CTD of the surface glycoprotein of the BVₚₘ. In particular embodiments of this type, the recombinant vector is a recombinant BVₚₘ, and the TMD and CTD of the surface glycoprotein originates from a virus species that is different from that of the recombinant BVₚₘ.

In order to more fully appreciate the invention, the following definitions are provided. The use of singular terms for convenience in description is in no way intended to be so limiting. Thus, for example, reference to a composition comprising “a polypeptide” includes reference to one or more of such polypeptides. In addition, reference to an “alphavirus RNA replicon particle” includes reference to a plurality of such alphavirus RNA replicon particles, unless otherwise indicated.

As used herein the term “approximately” is used interchangeably with the term “about” and signifies that a value is within fifty percent of the indicated value i.e., a composition containing “approximately” 1×10⁸ alphavirus RNA replicon particles per milliliter contains from 5×10⁷ to 1.5×10⁸ alphavirus RNA replicon particles per milliliter.

As used herein, a “recombinant vector” is a vector that is capable of introducing a heterologous gene into an isolated host cell or a host cell of a host organism, to produce the protein encoded by that heterologous gene. The host cell can be in a target animal. Examples of recombinant vectors include recombinant expression vectors and synthetic messenger RNAs.

As used herein, a “recombinant expression vector” is a recombinant vector that contains the appropriate signals to allow the expression of the encoded protein, e.g., a chimeric coronavirus spike protein, under suitable conditions in the host cell or host organism. Examples of recombinant expression vectors include DNA expression plasmids and recombinant viruses, including recombinant mammalian and avian viruses, RNA replicons, and RNA replicon particles.

A DNA expression plasmid is one type of recombinant expression vector that can be used to introduce a heterologous gene into a host cell or host organism to produce the protein encoded by that heterologous gene. The DNA expression plasmid can then be inserted into a eukaryotic host cell or eukaryotic host organism by some method of transfection, e.g., using a biochemical substance as carrier, by mechanical means, or by electroporation. Typically, the expression of the heterologous protein will be transient, as the DNA expression plasmid lacks signals for stable integration into the genome of a host cell. Consequently, a DNA expression plasmid will not transform or immortalize the host cell or the host organism. Examples of DNA expression plasmid are: pcDNA™, pCR3.1™ pCMV™, pFRT™, pVAX1™, pCI™, Nanoplasmid™, pFRT™ and pCAGGS [Niwa et al., Gene, 108:193-199 (1991)].

As used herein, the term “RNA replicon”, is used interchangeably with the term “replicon RNA” or “Replicon RNA” and refers to a modified RNA viral genome that lacks one or more elements (e.g., coding sequences for structural proteins) that if they were present, would enable the successful propagation of the parental virus in cell cultures or animal hosts. In suitable cellular contexts, the RNA replicon will amplify itself and may produce one or more sub-genomic RNA species. In contrast to an RNA replicon particle, an RNA replicon is not packaged with viral structural proteins and consequently, is less efficient at entering host cells.

As used herein, the term “RNA replicon particle”, abbreviated “RP” is an RNA replicon packaged in structural proteins e.g., the capsid and glycoproteins, which are derived from a virus. As used herein, the term “alphavirus RNA replicon particle” is an alphavirus-derived RNA replicon packaged in structural proteins, e.g., the capsid and glycoproteins, which also are derived from an alphavirus, e.g., as described by Pushko et al., [supra]. An RNA replicon particle cannot propagate in cell cultures or animal hosts (without a helper plasmid or analogous component), because the RNA replicon does not encode the alphavirus structural components (e.g., capsid and glycoproteins).

As used herein, the term “synthetic messenger RNA” or “synthetic mRNA” refers to a recombinant single-stranded molecule of mRNA that is constructed to comprise a nucleotide sequence of mRNA that encodes a chosen protein, flanked by 5′- and 3′-untranslated regions (UTRs) that stabilize mRNA and increase protein translation, thereby resembling a mature mRNA molecule as it occurs naturally in the cytoplasm of eukaryotic cells. These regulatory sequences can be derived from viral or eukaryotic genes. For the present invention, the synthetic mRNA comprises a nucleotide sequence that encodes a chimeric coronavirus spike protein. The synthetic messenger RNA is read by a ribosome in the process of synthesizing the chimeric coronavirus spike protein of the present invention. Typically, the 5′-UTR of the synthetic mRNA comprises a “5′ cap “structure, such as an 5′ RNA m⁷G cap, which is a modified guanine nucleotide that in nature is added to the 5′ end of a eukaryotic messenger RNA shortly after the start of transcription. The 5′ cap may consist of a terminal 7-methylguanosine residue that is linked through a 5′-5′-triphosphate bond to the first transcribed nucleotide. Its presence is critical for recognition by the ribosome and protection from RNases. A synthetic mRNA also typically has a 3′ poly-A tail, which is a covalent linkage of a polyadenylyl moiety to a messenger RNA molecule at the 3′ end. A synthetic mRNA can be delivered to a eukaryotic host organism or host cell by way of transfection and/or by using an appropriate carrier, e.g., a polymer or a cationic lipid. In contrast to the cap and the poly(A) tail, which are normally essential for mRNA stability and initiation of translation, the presence of other nuclear export signals found in naturally occurring mRNA is not required for synthetic mRNA vectors, as they are designed to be exclusively present in the cytoplasm. Details regarding the various structures of synthetic messenger RNA molecules to be used in the present invention and their synthesis are well known in the art [e.g., see review Pardi et al., Nat Rev Drug Discov 17:261-279, doi:10.1038/nrd.2017.243 (2018)]. Similarly, the delivery of synthetic mRNA directly into the cytoplasm necessitates its synthesis in a spliced form, allowing the possibility of the redundant splicing signals found in natural mRNA to be omitted in synthetic mRNA. [See, Tolmachov and Tolmachova, Gene Technology, 4(1):100017 (2015), U.S. Pat. No. 9,428,535, and Sclake et al. RNA Biol. 9(11): 1319-1330 (2012) doi: 10.4161/rna.22269.]

The synthetic mRNA as defined above can be in the form of a naked mRNA molecule or in a form wherein the mRNA molecule is associated with- or complexed to one or more carrier molecules that facilitate the cellular uptake of the synthetic mRNA molecule. A great variety of in vivo transfection reagents have been developed for this purpose (e.g., see review Pardi et al., supra).

As used herein “Y¹¹⁴⁴A” denotes a modification to the amino acid sequence of an IBV coronavirus spike protein that comprises the amino acid sequence of SEQ ID NO: 2. Accordingly, the tyrosine residue (Y) at position 1144 of SEQ ID NO: 2, which is in the CTD, is replaced with an alanine residue (A). This amino acid substitution functionally removes the ER-retention signal in the CTD of the IBV coronavirus spike protein. In the unmodified IBV coronavirus spike protein, the ER-retention signal serves to retain the spike protein in the ER or other intracellular compartments.

As used herein, a budding virus that buds from a host cell's plasma membrane is denoted as a “BVₚₘ” and is a virus that preferentially buds from the plasma membrane, but which may also less preferentially bud from intracellular compartments like endoplasmic reticulum (ER), endoplasmic reticulum-golgi intermediate compartment and the trans-Golgi network. Accordingly, a BVₚₘ is a virus that naturally exits the host cell by budding from the host cell's plasma membrane. Such budding from the host cell's plasma membrane enables a BVₚₘ to exit the host cell and is mostly used by enveloped viruses which must acquire a host-derived membrane enriched in viral proteins to form their external envelope. A BVₚₘ of the present invention is preferably an animal virus, e.g., an avian or mammalian virus. Examples of a BVₚₘ are VSV, influenza virus, NDV, HIV, Lassa virus, Ebola virus, and MV. Notably, coronavirus is not a BVₚₘ. Coronavirus spike proteins contain an ER-retention signal in the CTD, which retains the spike protein in the ER or other intracellular compartments [see, Welsch et al., Febs Letters 581:2089-2097 (2007), and Winter et al., J. Virol. 82(6):2765-27771 (2008)].

Detailed structural information on BVₚₘ surface glycoproteins, including their TMDs and CTDs, can be found in the various public nucleic acid- and protein sequence databases, such as the NCBI genome database, UniProt and EMBL/GenBank.

The terms “originate from”, “originates from” and “originating from” are used interchangeably with respect to a given protein or portion of that protein and the pathogen or strain of that pathogen that naturally encodes it, and as used herein signify that the unmodified and/or truncated amino acid sequence of that given protein or portion of that protein that is encoded by that pathogen or strain of that pathogen. The coding sequence, within a nucleic acid construct of the present invention for a protein or portion of that protein originating from a pathogen may have been genetically manipulated so as to result in a modification and/or truncation of the amino acid sequence of the expressed protein relative to the corresponding sequence of that protein in the pathogen or strain of pathogen (including naturally attenuated strains) it originates from.

A “surface glycoprotein” of a virus is a glycoprotein found on the surface of the viral envelope that serves to identify and bind to receptor sites on the host's membrane. The viral envelope then fuses with the host's membrane, allowing the capsid and viral genome to enter and infect the host. Examples of surface glycoproteins include the spike protein of coronaviruses and the surface glycoprotein of vesicular stomatitis virus.

VSV is a non-segmented negative-strand RNA virus that is in the Rhabdoviridae family, which includes rabies virus. VSV buds preferentially from the basolateral surface of polarized epithelial cells. This budding preference correlates with the basolateral localization of its glycoprotein [see, e.g., Drokhlyansky et al., J. Virol., 89(22):11718-11722 (2015)]. Such plasma membrane budding enables viruses to exit the host cell and is mostly used by enveloped viruses which must acquire a host-derived membrane enriched in viral proteins to form their external envelope.

IBV is a coronavirus, i.e., a member of the genus Gammacoronavirus, family Coronaviridae, of the order Nidovirales. The IBV S glycoprotein, i.e., spike protein, comprises about 1162 amino acid residues, and is cleaved into two subunits, S1 (about 535 amino acid residues and about a MW of 90-kDa) and S2 (about 627 amino acid residues and about a MW of 84-kDa). The C-terminal S2 subunit associates non-covalently with the N-terminal S1 subunit and contains the transmembrane and C-terminal cytoplasmic tail domains. The S1 subunit contains the receptor-binding activity of the spike protein. Furthermore, the IBV spike protein is involved in the induction of a protective immune response when inoculated into chickens [for a review see, Cavanagh, Vet. Res. 38:281-297 (2007); see also, EP0423869 A1; WO2004/078203 A2; and WO2012/110745 A2].

SARS-CoV-2 is a member of the genus Betacoronavirus, of the Coronaviridae family, of the order Nidovirales. The spike protein of a coronavirus is a large glycoprotein protruding from the surface of the virus that determines the tropism of the virus by binding to a specific extracellular domain of a host receptor. Human angiotensin-converting enzyme 2 (ACE2) serves as the host receptor for both the SARS-CoV-2 and the SARS-CoV spike proteins. The most variable part of the coronavirus genome is the RBD of coronavirus spike proteins. Notably however, five of the six critical amino acid residues of the RBD differ between the SARS-CoV-2 spike protein and the SARS-CoV spike protein. The SARS-CoV-2 spike protein further differs from a SARS-CoV spike protein by the SARS-CoV-2 spike protein comprising a polybasic cleavage site (RRAR, SEQ ID NO: 13) at the junction of the spike protein's two subunits, S1 and S2, whereas the SARS-CoV spike protein does not [see, Andersen et al., Nature Medicine 26:450-455 (2020)]. This polybasic cleavage site allows effective cleavage by proteases, which plays a role in the infectivity of SARS-CoV-2. Although the polybasic cleavage site is not unique to the SARS-CoV-2 spike protein, as the spike proteins of some of other human Betacoronaviruses comprise such structures, like SARS-CoV, the spike protein of the most closely related bat coronaviruses also have not been found to comprise this polybasic cleavage site. Detailed structural information on spike proteins of animal and human coronaviruses, including their TMDs and CTDs, can be found in the various public nucleic acid- and protein sequence data bases, such as the NCBI genome database, UniProt and EMBL/GenBank.

As used herein, a “transmembrane domain” or “TMD” is a hydrophobic region of a protein that either is or is to be inserted into the cell membrane. The parts of either side of the transmembrane domain of the protein are on opposite sides of the membrane. [See, e.g., The Senses: A Comprehensive Reference, Masland et al., editors; 2^{nd} editions (2008)]. The transmembrane domain of a coronavirus spike protein resides near the carboxy terminal part, right next to the cytoplasmic tail at the carboxy terminal of the protein. Detailed structural information on TMDs of spike proteins of animal and human coronaviruses can be found in the various public nucleic acid- and protein sequence data bases, such as the NCBI genome database, UniProt and EMBL/GenBank.

As used herein the term “C-terminal domain” or “CTD” is used interchangeably with the term “cytoplasmic tail” or “CT”, and is the portion of a surface glycoprotein of an enveloped virus, e.g., a spike protein of coronaviruses, that projects into the cytoplasm. The CTD of a type I membrane glycoprotein is at the carboxy terminus of the surface glycoprotein. Detailed structural information on CTD of spike proteins of animal and human coronaviruses can be found in the various public nucleic acid- and protein sequence data bases, such as the NCBI genome database, UniProt and EMBL/GenBank.

As used herein the abbreviation “2P” denotes a pair of consecutive proline residues that are substituted for two consecutive amino acid residues at the beginning of the central helix of a surface glycoprotein of an enveloped virus, e.g., a spike protein of coronaviruses, to further stabilize the surface glycoprotein in the prototypical prefusion conformation. [See, Pallesen et al., supra]

A “chimeric protein” is a protein that is made up of parts of two or more proteins [see e.g., McQueen et al., Proc.Natl.Acad.Sci., 83:9318-9322 (1986)].

As used herein, a “chimeric coronavirus spike protein” is a protein that is made up of a portion of a coronavirus spike protein (CSP) and a portion of a surface glycoprotein of a BVₚₘ, e.g., a recombinant protein that comprises the two subunits of the coronavirus spike protein: S1, which includes the receptor binding domain of a coronavirus spike protein, and S2, together with the TMID and the CTD from a surface glycoprotein of a BVₚₘ in place of the TMD and the CTD of the coronavirus spike protein. In particular embodiments, the BVₚₘ is a vesicular stomatitis virus.

As used herein, the term “over the same range of amino acid residues” with respect to performing a percent identity determination in which a specific range of amino acid residues, has been provided for a defined amino acid sequence, e.g., amino acid residues 14-1211 of SEQ ID NO: 12, indicates that the determination of that percent identity is made over that specific amino acid range.

As used herein, a “furin cleavage site” of a coronavirus Spike protein is a polybasic furin cleavage site that allows effective cleavage by proteases, e.g., the host cell's furin, which plays a role in the infectivity of many coronavirus Spike proteins including the Spike proteins of IBV and SARS-CoV-2 [see, Andersen et al., Nature Medicine 26: 450-455 (2020)]. Notably, the spike proteins of some of the other human Betacoronaviruses do not comprise such structures, e.g., SARS-CoV, and the spike proteins of the most closely related bat coronaviruses also have not been found to comprise this polybasic cleavage site.

As used herein, an “inactivated furin cleavage site” or “AFCS” of a coronavirus Spike protein is a furin cleavage site of the coronavirus Spike protein that has been genetically modified, so as not be susceptible to cleavage by the host cell furin protease. In the examples below, the furin cleavage site has been inactivated for the spike protein of IBV, i.e., amino acid residues RRFRR at position 533 to 537 of SEQ ID NOs: 4 and 6, were mutated to AAFAA (SEQ ID NO: 14), and the furin cleavage site has been inactivated for the spike protein of SARS-CoV-2, amino acid residues RRAR at position 682 to 685 of SEQ ID NOs: 8 and 10, were mutated to AAAR (SEQ ID NO: 15).

The term “non-SARS-CoV-2”, is used to modify terms such as pathogen, and/or antigen or immunogenic fragment thereof to signify that the respective pathogen, and/or antigen is neither a SARS-CoV-2 nor a SARS-CoV-2 protein antigen or immunogenic fragment thereof and that a non-SARS-CoV-2 antigen does not originate from a SARS-CoV-2.

The term “non-IBV”, is used to modify terms such as pathogen, and/or antigen or immunogenic fragment thereof to signify that the respective pathogen, and/or antigen is neither an IBV nor an IBV protein antigen or immunogenic fragment thereof and that a non-IBV antigen does not originate from an IBV.

As used herein, the terms “modified live” and “attenuated” are used interchangeably with respect to a given live virus and/or a live micro-organism.

As used herein, the terms “protecting”, and/or “providing protection to”, and/or “eliciting protective immunity to”, and/or “aids in the prevention of a disease”, and/or “aids in the protection”, and/or “reduces viral load”, and/or “reduces viremia” do not require complete protection from any indication of infection. For example, “aids in the protection” can mean that the protection is sufficient such that, after challenge, symptoms of the underlying infection are at least reduced, and/or aid in the reduction of viral shedding, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, a “vaccine” is a composition that is suitable for application to an animal, e.g., a chicken, or a feline, (with the term animal including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a disease arising from an infection with a wild-type virus and/or wild-type micro-organism, i.e., strong enough for aiding in the prevention of the disease, and/or preventing, ameliorating or curing the disease.

As used herein, “sterile immunity” is the type of immunity that prevents detectable replication of a particular disease-causing pathogen, such as SARS-CoV-2 (or particular strains thereof) and therefore prevents the establishment of a productive infection in an animal by that particular disease-causing pathogen.

As used herein a vaccine that “induces sterile immunity” in an animal against a particular disease-causing pathogen, such as SARS-CoV-2 (or particular strains thereof) through vaccination means that as a result of the vaccination, the vaccinated animal attains sterile immunity to that particular disease-causing pathogen. Inducing sterile immunity may require more than a single vaccine administration.

As used herein a vaccine that “prevents the transmission of coronavirus” means that the immune response in the vaccinated animal against a particular disease-causing pathogen, such as SARS-CoV-2 (or particular strains thereof) reduces the amount of replication of that particular disease-causing pathogen in the vaccinated animal to the extent that any shed of the particular disease-causing pathogen is insufficient for causing disease in other animals.

As used herein the term “mammal” is a vertebrate animal in which the young are nourished with milk from special mammary glands of the mother. Examples of mammals include humans, canines, felines, ovines, ferrets, and porcines.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, such as any member of the subfamilies Felinae, e.g., cats, lions, tigers, pumas, jaguars, leopards, snow leopards, panthers, North American mountain lions, cheetahs, lynx, bobcats, caracals or any cross breeds thereof. Cats also include domestic cats (Felis catus) including pure-bred and/or mongrel companion cats, show cats, laboratory cats, cloned cats, and wild or feral cats.

As used herein, a “ferret” is a mammal that is one of the mammals that belong to the mustelid family.

Typically, a vaccine of the present invention is administered in an amount effective, i.e., “effective amount”, that aids in the protection of the vaccinated animal from a coronavirus; e.g., aid in the protection of a human or feline from SARS-CoV-2, aids in the prevention of viral shedding in a feline or ferret, or aid in the protection of an avian from IBV.

As used herein, a multivalent vaccine is a vaccine that comprises two or more different antigens. In a particular embodiment of this type, the multivalent vaccine stimulates the immune system of the recipient against two or more different pathogens.

The terms “adjuvant” and “immune stimulant” are used interchangeably herein, and are defined as one or more substances that cause stimulation of the immune system. In this context, an adjuvant is used to enhance an immune response to one or more vaccine antigens/isolates. Accordingly, “adjuvants” are agents that nonspecifically increase an immune response to a particular antigen, thus reducing the quantity of antigen necessary in any given vaccine, and/or the frequency of injection necessary in order to generate an adequate immune response to the antigen of interest. In this context, an adjuvant is used to enhance an immune response to one or more vaccine antigens/isolates.

As used herein, a “nonadjuvanted vaccine” is a vaccine or a multivalent vaccine that does not contain an adjuvant.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient animal, e.g., a feline.

The term “carrier” refers to a diluent, adjuvant, excipient, or vehicle with which the recombinant vectors, e.g., alphavirus RNA replicon particles, are administered. Pharmaceutically acceptable carriers can be sterile liquids, such as water and/or oils, including those of petroleum-, animal-, vegetable- or synthetic origin, such as peanut oil, soybean oil, mineral oil, sesame oil and the like. Water or aqueous solution saline solutions and aqueous sugar, e.g., dextrose and/or glycerol solutions can be employed as carriers, particularly for injectable solutions. In the case of nonadjuvanted vaccines, the carrier cannot be an adjuvant.

“Parenteral administration” includes subcutaneous injections, submucosal injections, intravenous injections, intramuscular injections, intradermal injections, oral, intranasal, and infusion.

As used herein the term “immunogenic fragment” in regard to a particular protein (e.g., a protein antigen) is a fragment of that protein that is immunogenic, i.e., capable of specifically interacting with an antigen recognition molecule of the immune system, such as an immunoglobulin (antibody) or T cell antigen receptor. Preferably, an immunogenic fragment of the present invention is immunodominant for antibody and/or T cell receptor recognition. In particular embodiments, an immunogenic fragment with respect to a given protein antigen, is a fragment of that protein that retains at least 25% of the antigenicity of the full-length protein SARS-CoV-2 spike protein or the IBV spike protein. In preferred embodiments an immunogenic fragment retains at least 50% of the antigenicity of the full-length protein SARS-CoV-2 spike protein or the IBV spike protein. In more preferred embodiments, an immunogenic fragment retains at least 75% of the antigenicity of the full-length protein SARS-CoV-2 spike protein or the IBV spike protein. Immunogenic fragments can be 100 amino acid residues or more that comprise at least one conserved region of the full-length chimeric spike protein or at the other extreme, be large fragments that are missing as little as a single amino acid from the full-length protein. In particular embodiments, the immunogenic fragment comprises 125 to 1000 amino acid residues of the full-length protein chimeric spike protein. In other embodiments, the immunogenic fragment comprises 250 to 750 amino acid residues of the full-length chimeric spike protein.

As used herein one amino acid sequence is 100% “identical” or has 100% “identity” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, or in the case of a chimeric protein: the portion of the polypeptide being compared.

Accordingly, the percent identity of a chimeric coronavirus spike protein of the present invention is individually performed for each of the different proteins in the chimeric spike protein. For example, in the case of a chimeric coronavirus spike protein of the present invention that is made up of: (i) all of an IBV spike protein except the TMD and the CTD of the IBV spike protein, and (ii) only the TMD and CTD of the surface protein of a vesicular stomatitis virus, the amino acid sequence comparison for the IBV spike protein is over the amino acid sequence of the chimeric coronavirus spike protein originating from the IBV spike protein (generally without the signal sequence) and the amino acid sequence comparison for the surface protein of a vesicular stomatitis virus is over the amino acid sequence of the chimeric coronavirus spike protein originating from the surface protein of a vesicular stomatitis virus protein. Again, the determination of the percent identity of the portion of the coronavirus spike protein is performed over a contiguous block of amino acid residues comprised by the corresponding portion of the protein.

In a particular embodiment, selected deletions or insertions that could otherwise alter the correspondence between the two amino acid sequences are taken into account. Importantly, a chimeric coronavirus spike protein comprising a defined percent (%) or greater identity with a defined amino acid sequence of a chimeric coronavirus spike protein of the present invention, must retain the specified functional properties of that defined amino acid sequence of the chimeric coronavirus spike protein. Accordingly, a chimeric coronavirus spike protein comprising a percent or greater identity with the defined amino acid sequence of a chimeric coronavirus spike protein of the present invention, in which the furin cleavage site of the chimeric coronavirus spike protein is inactivated, must retain the property of having an inactivated cleavage site despite the remaining variability of the overall amino acid sequence. Similarly, a chimeric coronavirus spike protein that is further stabilized in a prefusion state due to the replacement of two consecutive amino acid residues at the beginning of the central helix of the coronavirus spike protein by a pair of proline residues (2P), must retain this pair of proline residues despite the remaining variability of the overall amino acid sequence.

As used herein, nucleotide and amino acid sequence percent identity can be determined using C, MacVector™ (MacVector, Inc. Cary, NC 27519), Vector NTI™ (Informax, Inc. MD), Oxford Molecular Group PLC (1996), and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) Pileup program using the default parameters.

For the purposes of this invention, an “inactivated” virus or microorganism is a virus or micro-organism which is capable of eliciting an immune response in an animal, but is not capable of infecting the animal. For example, an inactivated SARS-CoV-2 may be inactivated by an agent selected from the group consisting of binary ethyleneimine, formalin, beta-propiolactone, thimerosal, or heat.

### Recombinant Vectors

A “vector” is well-known in the field of the invention as a molecular structure that carries the genetic information (a nucleotide sequence), for encoding a polypeptide, with appropriate signals to allow its expression under suitable conditions, such as in a host cell. For the invention ‘expression’ regards to the well-known principle of the expression of protein from genetic information by way of transcription and/or translation. Many types and variants of a recombinant vector are known and can be used in the present invention, ranging from nucleic acid molecules like DNA or RNA, to more complex structures such as virus-like particles and replicon particles, up to replicating recombinant micro-organisms such as a recombinant viral vector. Depending on the type of vector employed more or less expression signals need to be provided, either in cis (i.e., provided within the recombinant vector itself) or in trans (i.e., provided from a separate source).

A “recombinant vector” for the invention, is a vector of which the genetic constitution does not fully match with that of its native counterpart. Such a vector thus has a molecular make-up that was changed, typically by manipulation in vitro of its genetic information by way of molecular cloning, and recombinant protein expression techniques. The changes made can serve to provide for, to improve or to adapt the expression, manipulation, purification, stability and/or the immunological behavior of the vector and/or of the protein it expresses. These, and other techniques are explained in great detail in standard text-books, [Sambrook and Russell, “Molecular cloning: a laboratory manual: Cold Spring Harbour Laboratory Press (2001); ISBN: 0879695773); Ausubel et al., in: Current Protocols in Molecular Biology, (J. Wiley and Sons Inc, NY, (2003), ISBN: 047150338X); Dieffenbach and Dveksler: “PCR primers: a laboratory manual” (CSHL Press, ISBN 0879696540); and Bartlett and Stirling, “PCR Protocols”, (Humana press, ISBN: 0896036421)].

One type of recombinant vector is a recombinant expression vector, which includes recombinant viral vectors such as recombinant HVT vectors, which are predominantly used in chicken vaccines [see e.g., U.S. Pat. No. 5,853,733] and RNA Replicon Particles, which have a broader range of animal subjects [see e.g., Pushko et al., supra].

Recombinant Herpesvirus of Turkey Vectors

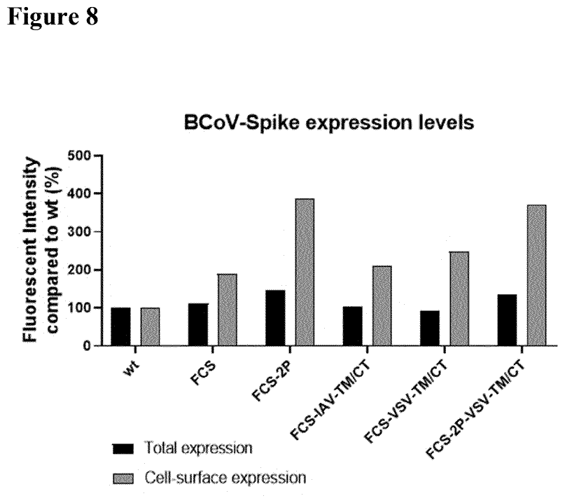

The ability to generate herpesviruses by co-transfection of cloned overlapping subgenomic fragments was first demonstrated for pseudorabies virus [van Zijl et al., J. Virology 62:2191-2195 (1988)]. This procedure subsequently was employed to construct recombinant HVT vectors [see, U.S. Pat. No. 5,853,733, hereby incorporated by reference with respect to the methodology disclosed regarding the construction of recombinant HVT vectors] and can be used to construct the recombinant HVT vectors encoding the chimeric coronavirus spike proteins of the present invention. In this method, the entire HVT genome is cloned into bacterial vectors as several large overlapping subgenomic fragments constructed utilizing standard recombinant DNA techniques [Maniatis et al., (1982) Molecular Cloning, Cold Spring Harbor Laboratory Press, Cold Spring Harbor, New York (1982); and Sambrook et al., Molecular Cloning, Cold Spring Harbor Laboratory Press, Cold Spring Harbor, New York (1989)]. An HVT strain FC126 cosmid library was derived from sheared viral DNA cloned into the cosmid vector, pWE15 (Stratagene, now Agilent Technologies of Santa Clara, CA). In addition, several large genomic DNA fragments were isolated by restriction digestion with the enzyme, BamHI, and cloned into either pWE15 or the plasmid vector pSP64 (Promega, Madison WI). As described in U.S. Pat. No. 5,853,733, co-transfection of these fragments into chicken embryo fibroblast (CEF) cells results in the regeneration of the HVT genome mediated by homologous recombination across the overlapping regions of the fragments. If an insertion is engineered directly into one or more of the subgenomic fragments prior to the co-transfection, this procedure results in a high frequency of viruses containing the insertion. For example, five overlapping subgenomic clones were required to generate FC126 HVT and served as the basis for creating a series of HVT/NDV/ILTV recombinant viruses [see, U.S. Pat. No. 8,932,064 B2]. The cosmid regeneration recombinant HVT constructs can be performed essentially as described in U.S. Pat. No. 5,853,733 [see e.g. FIG. 8 of U.S. Pat. No. 5,853,733]. Alternatively, desired recombinant avian herpesvirus viruses also can be constructed using the CRISPR/Cas9 system [see, Tang et al., Vaccine, 36(5):716-722 (2018)].

Recombinant RNA Viruses, RNA Replicons, and RNA Replicon Particles

RNA viruses can be used as vector-vehicles for introducing nucleotides encoding a vaccine antigen, e.g., a nucleotide sequence encoding a chimeric coronavirus spike protein of the present invention, that has been genetically engineered into their genomes. However, their use to date has been limited primarily to incorporating viral antigens into the RNA virus and then introducing the virus into a recipient host. The result is the induction of protective antibodies against the incorporated viral antigens. Alphavirus RNA replicon particles have been used to encode pathogenic antigens. Such alphavirus replicon platforms have been developed from several different alphaviruses, including VEEV [Pushko et al., supra], Sindbis (SIN) [Bredenbeek et al., Journal of Virology 67:6439-6446 (1993) the contents of which are hereby incorporated herein in their entireties], and Semliki Forest virus (SFV) [Liljestrom and Garoff, Biotechnology (NY) 9:1356-1361 (1991), the contents of which are hereby incorporated herein in their entireties]. Moreover, alphavirus RNA replicon particles are the basis for several USDA-licensed vaccines for swine and poultry. These include: Porcine Epidemic Diarrhea Vaccine, RNA Particle (Product Code 19U5.P1), Swine Influenza Vaccine, RNA (Product Code 19A5.D0), Avian Influenza Vaccine, RNA (Product Code 1905.D0), and Prescription Product, RNA Particle (Product Code 9PP0.00).

The alphavirus RNA replicon particles of the present invention may be lyophilized and rehydrated with a sterile water diluent. On the other hand, when the alphavirus RNA replicon particles are stored separately, but intended to be mixed with other vaccine components prior to administration, the alphavirus RNA replicon particles can be stored in the stabilizing solution of those components, e.g., a high sucrose solution.

Accordingly, in one aspect of the present invention, the vaccines comprise alphavirus RNA RPs that comprise the capsid protein and glycoproteins of VEEV. In even more specific embodiments, the vaccines comprise alphavirus RNA RPs that comprise the capsid protein and glycoproteins of the avirulent TC-83 strain of VEEV and encode a chimeric coronavirus spike protein. Immunogenic compositions and/or vaccines (including multivalent vaccines) comprising the alphavirus RNA replicon particles encoding the chimeric coronavirus spike protein can be administered in the presence or alternatively in the absence of an adjuvant. In certain embodiments, the immunogenic compositions and/or vaccines are for humans. In other embodiments, the immunogenic compositions and/or vaccines are for felines. In yet other embodiments, the immunogenic compositions and/or vaccines are for ferrets. In still other embodiments the immunogenic compositions and/or vaccines are for chickens. Methods of making and using the vaccines and/or immunogenic compositions alone or in combinations with other protective agents are also provided.

Promoters

Aside from using the native promoter of the given recombinant vector, e.g., a recombinant viral vector, to drive the expression of a heterologous gene encoding a protein antigen in a recombinant viral vector of the present invention, many alternative promoters also can be used in a recombinant viral vector e.g., the pseudorabies virus (PRV) gpX promoter [see, WO 87/04463], the Rous sarcoma virus LTR promoter, the SV40 early gene promoter, the human cytomegalovirus immediate early 1 (hCMV IE1) gene promoter [U.S. Pat. Nos. 5,830,745; 5,980,906], and the chicken beta-actin gene promoter [EP 1 298 139 B1]. The inclusion of a polyadenylation regulatory element downstream from a nucleotide coding region is oftentimes required to terminate the transcription of the coding nucleotide sequence. Accordingly, many genes comprise a polyadenylation regulatory element at the downstream end of their coding sequence. Many such regulatory elements have been identified and can be used in a recombinant expression vector of the present invention.

Synthetic Messenger RNA

Production of synthetic mRNA encoding a chimeric coronavirus spike protein of the present invention can begin by plasmid DNA linearization using a restriction enzyme prior to in vitro run-off transcription using, for example, the MegaScript® T7 RNA polymerase and cap analog. (This process is analogous to that used for the RNA transcription found in RNA replicon production). The synthetic mRNA molecule should be packaged so as to be protected from RNAses and for efficient delivery in eukaryotic cells. For the delivery, different technologies can be used such as cationic polymers, dendrimers, or lipid nanoparticles (LNPs). [See e.g., Pardi et al., supra] A synthetic mRNA for use as a recombinant vector can be delivered to its target animal or to a host cell in a number of ways including by mechanical or chemical means, by transfection, or encapsulated with an appropriate (nanoparticulate) carrier, such as a protein, polysaccharide, cationic lipid, or a polymer. To stabilize the synthetic mRNA, certain chemical modifications may be applied e.g., to the nucleotides, to their backbone, or by incorporation of the nucleotide-analogues. [See e.g., U.S. Pat. No. 9,447,164.]

### Vaccines and Multivalent Vaccines

The present invention further provides vaccines that comprise a recombinant vector of the present invention and a pharmaceutically acceptable carrier. In one aspect of the invention, the vaccine aids in the protection of a human, a feline, or a ferret from an infection by SARS-CoV-2. In particular embodiments of this type, the vaccine aids in reducing shedding of SARS-CoV-2 in a feline. The present invention further provides vaccines that aid in reducing shedding of SARS-CoV-2 in a ferret. In other embodiments, the feline vaccines aid in reducing the severity of one or more clinical signs in the infected feline. In still other embodiments, the ferret vaccines aid in reducing the severity of one or more clinical signs in the infected ferrets. In yet other embodiments the vaccine aids in the protection of a chicken.

The present invention also provides multivalent vaccines and immunogenic compositions. Any antigen or combination of such antigens useful in a mammalian or alternatively, in an avian immunogenic composition or vaccine, can be added to any respective mammalian vaccine or immunogenic composition, or avian vaccine or immunogenic composition respectively, of the present invention. Such multivalent vaccines and/or immunogenic compositions are included in the present invention. In particular embodiments, a multivalent vaccine comprising an alphavirus RNA RP that encodes a chimeric SARS-CoV-2 spike protein and one or more other SARS-CoV-2 protein antigens, and/or one or more non-SARS-CoV-2 protein antigens, and/or further comprises one or more additional alphavirus RNA replicon particles that encode, e.g., one or more other SARS-CoV-2 protein antigens, and/or one or more non-SARS-CoV-2 protein antigens. In similar embodiments, a multivalent vaccine comprising an alphavirus RNA RP that encodes one or more chimeric coronavirus spike proteins, e.g., a chimeric IBV spike protein, further comprises one or more additional alphavirus RNA replicon particles that encode, e.g., one or more other one or more non-IBV protein antigens.

Accordingly, the avian vaccines of the present invention comprising a recombinant vector that encodes a chimeric IBV spike protein of the present invention can further comprise at least one non-IBV antigen for eliciting protective immunity to a non-IBV pathogen. In certain embodiments of this type, the vaccines further comprise a recombinant vector comprising a nucleotide sequence encoding at least one antigen or immunogenic fragment thereof that originates from the non-IBV pathogen. In particular embodiments of this type, the recombinant vector is an HVT. In alternative embodiments, the recombinant vector is a VEEV RNA replicon particle.

Accordingly, in certain embodiments, the recombinant vectors are recombinant viral vectors that further encode one or more other antigens. In particular embodiments of this type, the recombinant viral vectors further encode a second IBV protein antigen. In more particular embodiments, the second IBV protein antigen is a second chimeric IBV spike protein that comprises an IBV spike protein that originates from a different strain of IBV than the first chimeric IBV spike protein originates from. In other embodiments, the recombinant vectors can encode a first chimeric IBV spike protein, optionally together with the second chimeric IBV spike protein, and one or more antigens from a non-IBV. In certain embodiments, the non-IBV antigen is a NDV antigen. In certain embodiments of this type the NDV antigen is a F protein. In yet other embodiments the non-IBV antigen is an Infectious Bursal Disease Virus (IBDV) antigen. In certain embodiments of this type, the IBDV antigen is a viral protein 2 (VP2). In still other embodiments, the non-IBV antigen is an Infectious Laryngotracheitis Virus (ILTV) protein. In certain embodiments of this type, the ILTV protein is a glycoprotein B (gB). In other such embodiments, the ILTV protein is a glycoprotein D (gD). In still other embodiments, the ILTV protein is a glycoprotein I (gI). In yet other embodiments, the recombinant viral vector encodes any combination of two or more of the ILTV gD, gI, and gB. In other embodiments the non-IBV antigen is an Avian Influenza Virus (AIV) protein. In certain embodiments of this type, the AIV protein is an AIV hemagglutinin (HA). In other embodiments, the AIV protein is an AIV neuraminidase (NA). In yet other embodiments, the recombinant viral vector encodes both an AIV HA and an AIV NA.

For example, a recombinant HVT could be constructed to encode and express a chimeric IBV spike protein either alone or in a multivalent HVT vector that includes e.g., one or more avian influenza antigens. Multivalent HVT vectors are well known in the field [see, e.g., U.S. Pat. No. 8,932,064 B2]. In yet other embodiments, the recombinant viral vector can be a recombinant attenuated MDV1. In still other embodiments, the recombinant viral vector can be a recombinant attenuated MDV2. In yet other embodiments, the recombinant viral vector can be a recombinant attenuated NDV.

Similarly, a recombinant vector that encodes a chimeric IBV spike protein in an avian vaccine can be added together with one or more live, attenuated virus isolates, e.g., a live attenuated NDV, and/or a live attenuated IBDV, and/or a live attenuated ILTV, and/or a live attenuated Marek's Disease Virus (MDV), including HVT, a naturally attenuated virus, and/or a live attenuated avian influenza virus (AIV).

In alternative vaccine embodiments, the non-IBV antigen is an inactivated non-IBV pathogen. In particular vaccine embodiments, the non-IBV pathogen can be an inactivated NDV. In other vaccine embodiments, the non-IBV pathogen is an inactivated IBDV. In yet other vaccine embodiments, the non-IBV pathogen is an inactivated ILTV. In still other vaccine embodiments, the non-IBV pathogen is an inactivated MDV1. In yet other vaccine embodiments, the non-IBV pathogen is an HVT. In still other vaccine embodiments, the non-IBV pathogen is an inactivated avian influenza virus. In certain vaccine embodiments, the vaccines comprise non-IBV antigens from multiple non-IBV pathogens.

Multivalent mammalian vaccines and/or immunogenic compositions which comprise a recombinant vector encoding both a chimeric SARS-CoV-2 spike protein and a non-SARS-CoV-2 pathogen antigen are included in the present invention. In particular vaccine embodiments, the non-SARS-CoV-2 pathogen is a feline calicivirus (FCV). In other vaccine embodiments, the non-SARS-CoV-2 pathogen is a feline leukemia virus (FeLV). In yet other vaccine embodiments, the non-SARS-CoV-2 pathogen is a feline panleukopenia virus (FPLV). In still other vaccine embodiments, the non-SARS-CoV-2 pathogen is a feline rhinotracheitis virus (FVR). In yet other vaccine embodiments, the non-SARS-CoV-2 pathogen is a feline immunodeficiency (FIV). In particular vaccine embodiments, the non-SARS-CoV-2 pathogen is a Chlamydophila felis. In still other vaccine embodiments, the non-SARS-CoV-2 pathogen is a canine influenza virus (CIV). In yet other vaccine embodiments, the non-SARS-CoV-2 pathogen is a canine parvovirus (CPV). In still other vaccine embodiments, the non-SARS-CoV-2 pathogen is a canine distemper virus (CDV). In yet other vaccine embodiments, the non-SARS-CoV-2 pathogen is a rabies virus. In certain vaccine embodiments, the vaccines comprise antigens from non-SARS-CoV-2 antigens from multiple non-SARS-CoV-2 pathogens.

Moreover, an alphavirus RNA RP that encodes one or more chimeric coronavirus spike proteins, e.g., a chimeric SARS-CoV-2 spike protein, in a human, feline, or ferret vaccine and/or corresponding immunogenic composition can be added together with one or more other inactivated virus isolates, e.g., such as an inactivated FCV strain, and/or an inactivated feline herpesvirus and/or an inactivated feline parvovirus and/or an inactivated feline leukemia virus, and/or an inactivated feline infectious peritonitis virus and/or an inactivated feline immunodeficiency virus, and/or an inactivated rabies virus, and/or an inactivated feline influenza virus, and/or an inactivated canine influenza virus. In addition, bacterins (or subfractions of the bacterins, e.g., the pilus subfraction) of Chlamydophila felis, and/or Bordetella bronchiseptica and/or Bartonella spp. (e.g., B. henselae) can also be included in such multivalent vaccines.

Moreover, an alphavirus RNA RP that encodes a chimeric coronavirus spike protein in a human, feline, or ferret immunogenic composition and/or vaccine can be added together with one or more live, attenuated virus isolates, e.g., a live attenuated FCV virus and/or a live, attenuated feline leukemia virus, and/or a live, attenuated feline infectious peritonitis virus and/or a live, attenuated feline immunodeficiency virus, and/or a live, attenuated rabies virus, and/or a live, attenuated feline influenza virus and/or a live, attenuated canine influenza virus. In addition, a live, attenuated Chlamydophila felis, and/or a live, attenuated Bordetella bronchiseptica and/or a live, attenuated Bartonella spp. (e.g., B. henselae) can also be included in such multivalent vaccines.

Accordingly, the present invention provides vaccines comprising one or more VEEV RNA replicon particles, which encode a second SARS-CoV-2 protein antigen. In particular embodiments, a first VEEV RNA replicon particle encodes a first chimeric SARS-CoV-2 spike protein and a second VEEV RNA replicon particle encodes a second chimeric SARS-CoV-2 spike protein that originates from a different strain of SARS-CoV-2 than the first SARS-CoV-2 spike protein originates from.

In particular vaccines, the recombinant viral vector is an alphavirus RNA replicon particle. In more particular embodiments of this type, the alphavirus RNA replicon particle is a VEEV RNA replicon particle. In even more particular embodiments, the vaccines comprise alphavirus RNA RPs that comprise the capsid protein and glycoproteins of the avirulent TC-83 strain of VEEV and encode a chimeric coronavirus spike protein.

Adjuvants:

In one aspect of the present invention, the vaccines are non-adjuvanted, i.e., do not comprise an adjuvant. On the other hand, in certain embodiments the vaccines do contain an adjuvant. Examples of adjuvants that may be used in the vaccines of the present invention include CARBOPOL®[e.g., polymers of acrylic acid cross-linked with polyalkenyl ethers or divinyl glycol, Alhydrogel+QuilA, aluminium hydroxide, Alhydrogel, Emulsigen+EMA31+Neocryl XK62, Carbomer, Carbomer 974P, Adjuphos, Alhydrogel+QS21 (saponin) Carbigen. In particular embodiments, the adjuvant is an oil adjuvant comprising more than one oil, e.g., a mineral oil and one or more non-mineral oils. In certain embodiments of this type the oil adjuvant comprises a liquid paraffin oil as the mineral oil, and one or more non-mineral oils selected from squalane, squalene, vitamin E, vitamin E-acetate, oleate, and ethyl-oleate. In more particular embodiments, the oil adjuvant comprises a liquid paraffin oil and vitamin E-acetate. In still more particular embodiments, the oil adjuvant is XSOLVE™

Administration:

A vaccine of the present invention can be readily administered by any standard route including by parenteral administration, and more particularly intravenous, intramuscular, subcutaneous, oral, intranasal, intradermal, and/or intraperitoneal vaccination. The artisan will appreciate that the vaccine composition is preferably formulated appropriately for each type of recipient animal and route of administration. Thus, the present invention also provides methods of immunizing a mammal against a coronavirus and/or other animal pathogens. One such method comprises injecting a mammal with an immunologically effective amount of a human, feline, or ferret vaccine of the present invention, so that the human, feline, or ferret produces appropriate antibodies to the SARS-CoV-2 spike protein. Another such method comprises injecting a chicken with an immunologically effective amount of an avian vaccine of the present invention, so that the chicken produces appropriate antibodies to an IBV spike protein. In this method, the “chicken” may be a chicken of any age. In an embodiment the chicken is an embryo, when applying a so-called in ovo method of immunizing a chicken.

The following examples serve to provide further appreciation of the invention, but are not meant in any way to restrict the effective scope of the invention.

Further Methods and Uses:

As outlined above, the recombinant vectors of the present invention can be used advantageously in vaccines or immunogenic compositions according to the invention, which can be manufactured by well-known methods. These aspects and embodiments can also be worded differently for different jurisdictions.

Therefore in a further aspect, the invention regards the recombinant vector according to the invention for use as a vaccine, wherein the vaccine is an aid in the protection of a mammal from an infection by SARS-CoV-2, or the vaccine is an aid in the protection of an avian from infectious bronchitis. In an embodiment of the recombinant vector for use as a vaccine, the recombinant vector is selected from the recombinant expression vector, the recombinant viral vector, the DNA expression plasmid, the alphavirus RNA replicon particle, and the synthetic mRNA, all as defined herein.

In a further aspect, the invention regards the use of the recombinant vector according to the invention for the manufacture of a vaccine, wherein the vaccine is an aid in the protection of a mammal from an infection by SARS-CoV-2, or the vaccine is an aid in the protection of an avian from infectious bronchitis. In an embodiment of the use of the recombinant vector for the manufacture of a vaccine, the recombinant vector is selected from the recombinant expression vector, the recombinant viral vector, the DNA expression plasmid, the alphavirus RNA replicon particle, and the synthetic mRNA, all as defined herein.

In a further aspect, the invention regards a method for the manufacture of the vaccine according to the invention, the method comprising the admixing of the recombinant vector according to the invention and a pharmaceutically acceptable carrier. In an embodiment of the method for the manufacture of the vaccine, the recombinant vector is selected from the recombinant expression vector, the recombinant viral vector, the DNA expression plasmid, the alphavirus RNA replicon particle, and the synthetic mRNA, all as defined herein.

### EXAMPLES

The following abbreviations are used in the labeling of the coronavirus spike proteins and chimeric coronavirus spike proteins employed in the Examples below and their respective nucleotide and amino acid sequences:
WT or wt: Wild type protein
SP: Signal peptide
RBD Receptor binding domain
ΔFCS: Inactivation of the furin cleavage site
ΔCTD: Removal of the CTD
VSV: Replacement of the TMD and the CTD of the Spike protein by the TMD and CTD of the surface glycoprotein of Vesicular Stomatitis Virus.
2P: The addition of the 2P modification to stabilize the prototypical prefusion conformation.
Y¹¹⁴⁴A: The functional removal of the ER-retention signal
3M: The addition of a trimerization domain

### Example 1

NUCLEOTIDE AND AMINO ACID SEQUENCES

IBV-Ma5-Spike [SEQ ID NO: 1]
atgctggtgaccccactgctgctggtgacactgctgtgcgcactgtgctccgccgctctgtacgatagctccagctacgt
gtactactaccagagcgcattccggccccctgatggatggcacctgcacggcggagcctacgctgtggtgaacatctcca
gcgagagcaacaatgctggctccagctccggatgcacagtggggatcattcacgggggcagagtggtgaatgcaagctcc
attgcaatgactgcccccagctccggaatggcatggagctccagccagttttgcaccgcctactgcaactttagcgatac
cacagtgttcgtgacacactgctacaagcacggagggtgcccaatcactggaatgctgcagcagcacagcattagggtgt
ccgcaatgaaaaacgggcagctgttctacaatctgacagtgagcgtggccaagtaccccacttttaaatccttccagtgc
gtgaacaatctgaccagcgtgtacctgaacggcgatctggtgtacaccagcaatgctactaccgacgtgacatccgcagg
agtgtactttaaggccggcggacctatcacatacaaagtgatgcgggaagtgagagcactggcctacttcgtgaatggca
ctgctcaggatgtgattctgtgcgatggctccccaaggggactgctggcatgccagtacaacaccggcaatttcagcgac
ggattttaccccttcacaaactccagcctggtgaagcagaaatttatcgtgtaccgcgagaacagcgtgaatacaacttt
cacactgcacaacttcacttttcacaatgaaaccggagccaaccccaatcctagcggggtgcagaacattcagacatacc
agacccagacagctcagagcgggtactacaacttcaatttttccttcctgtccagctttgtgtacaaggagagcaacttc
atgtacggcagctaccacccatcctgcaattttcggctggaaacaatcaacaatgggctgtggttcaactccctgagcgt
gtccattgcttacggccctctgcagggcggctgcaaacagagcgtgttttccggaagagccacctgctgctacgcttact
cctacggagggccactgctgtgcaagggggtgtacagcggcgagctggatcacaatttcgaatgcggactgctggtgtac
gtgaccaaaagcggcggcagcagaatccagactgccaccgagccacccgtgatcacacagcacaactacaacaatattac
actgaacacttgcgtggactacaatatctacgggagaactggccagggattcattaccaacgtgacagatagcgctgtgt
cctacaattacctggctgacgcaggcctggcaatcctggataccagcggcagcatcgacatttttgtggtgcagtccgag
tacggcctgaactactacaaagtgaatccctgcgaagatgtgaaccagcagttcgttgtgagcgggggcaaactggtggg
aatcctgacaagccggaatgagactgggtcccagctgctggaaaaccagttctacatcaagatcactaacggaaccagaa
ggttccgccggagcatcacagagtccgtggaaaactgcccttacgtgtcctacgggaagttttgcattaaaccagacggc
agcatcgccactattgtgcccaagcagctggagcagtttgtggctcctctgctgaacgtgaccgaaaatgtgctgatccc
aaacagcttcaatctgacagtgactgatgagtacattcagaccaggatggacaaagtgcagatcaactgcctgcagtaca
tttgcgggaacagcctggaatgccgcaatctgttccagcagtacggccctgtgtgcgataacatcctgagcgtggtgaac
agcgtgggccagaaggaggacatggaactgctgaacttttactccagcactaaacccgccggcttcaacacccctgtgct
gagcaatgtgtccaccggagagtttaatatctccctgttcctgaccacaccatccagccccagaaggcgcagctttattg
aggatctgctgttcacaagcgtggaatccgtggggctgcccactgatgacgcttacaagaactgcaccgcaggccctctg
ggattcctgaaagacctggcctgcgctcgcgagtacaatggcctgctggtgctgcctccaatcattacagctgaaatgca
gatcctgtacacttccagcctggtggctagcatggcatttggagggatcactgcagccggggcaattcccttcgccaccc
agctgcaggcaaggatcaaccacctgggcattacacagtccctgctgctgaagaaccaggagaaaatcgctgccagcttc
aataaggctattgggcacatgcaggaaggcttccgcagcacttccctggcactgcagcagatccaggatgtggtgaacaa
gcagtccgccattctgaccgagacaatggctagcctgaacaaaaattttggcgccatctccagcgtgatccaggaaattt
accagcagctggatgctatccaggcaaacgcccaggtggacaggctgattacaggacgcctgtccagcctgagcgtgctg
gcttccgcaaagcaggcagagtacatccgggtgtcccagcagagagagctggccacacagaagatcaacgaatgcgtgaa
aagccagtccattcggtacagcttctgcgggaatggcagacacgtgctgactatccctcagaacgccccaaatggcatcg
tgtttattcacttcagctacacccccgactcctttgtgaacgtgacagctatcgtgggattctgcgtgaagccagccaat
gcttcccagtacgctattgtgcctgcaaacggaagagggatctttattcaagtgaatggaagctactacatcactgcaag
ggatatgtacatgcctcgcgccatcaccgctggggacattgtgactctgaccagctgccaggccaactacgtgtccgtga
ataaaaccgtgatcactaccttcgtggataacgatgactttgatttcaatgacgagctgagcaagtggtggaacgacaca
aaacacgaactgcctgattttgacaagttcaattacactgtgccaatcctggatattgacagcgagatcgataggattca
gggagtgatccaggggctgaacgatagcctgattgacctggaaaaactgtccatcctgaagacatacattaaatggccct
ggtacgtgtggctggcaatcgcctttgctaccatcattttcatcctgattctgggatgggtgttctttatgacagggtgc
tgcggctgctgctgcggatgctttgggattatgcccctgatgagcaagtgcgggaagaaatccagctactacacaacttt
cgataacgacgtggtgaccgagcagtaccgccctaagaaaagcgtgtga
IBV-Ma5-Spike [SEQ ID NO: 2]
MLVTPLLLVTLLCALCSAALYDSSSYVYYYQSAFRPPDGWHLHGGAYAVVNISSESNNAGSSSGCTVGIIHGGRVVNASS
IAMTAPSSGMAWSSSQFCTAYCNFSDTTVFVTHCYKHGGCPITGMLQQHSIRVSAMKNGQLFYNLTVSVAKYPTFKSFQC
VNNLTSVYLNGDLVYTSNATTDVTSAGVYFKAGGPITYKVMREVRALAYFVNGTAQDVILCDGSPRGLLACQYNTGNFSD
GFYPFTNSSLVKQKFIVYRENSVNTTFTLHNFTFHNETGANPNPSGVQNIQTYQTQTAQSGYYNFNFSFLSSFVYKESNF
MYGSYHPSCNFRLETINNGLWENSLSVSIAYGPLQGGCKQSVESGRATCCYAYSYGGPLLCKGVYSGELDHNFECGLLVY
VTKSGGSRIQTATEPPVITQHNYNNITLNTCVDYNIYGRTGQGFITNVTDSAVSYNYLADAGLAILDTSGSIDIFVVQSE
YGLNYYKVNPCEDVNQQFVVSGGKLVGILTSRNETGSQLLENQFYIKITNGTRRFRRSITESVENCPYVSYGKFCIKPDG
SIATIVPKQLEQFVAPLLNVTENVLIPNSFNLTVTDEYIQTRMDKVQINCLQYICGNSLECRNLFQQYGPVCDNILSVVN
SVGQKEDMELLNFYSSTKPAGFNTPVLSNVSTGEFNISLFLTTPSSPRRRSFIEDLLFTSVESVGLPTDDAYKNCTAGPL
GFLKDLACAREYNGLLVLPPIITAEMQILYTSSLVASMAFGGITAAGAIPFATQLQARINHLGITQSLLLKNQEKIAASF
NKAIGHMQEGFRSTSLALQQIQDVVNKQSAILTETMASLNKNFGAISSVIQEIYQQLDAIQANAQVDRLITGRLSSLSVL
ASAKQAEYIRVSQQRELATQKINECVKSQSIRYSFCGNGRHVLTIPQNAPNGIVFIHFSYTPDSFVNVTAIVGFCVKPAN
ASQYAIVPANGRGIFIQVNGSYYITARDMYMPRAITAGDIVTLTSCQANYVSVNKTVITTFVDNDDEDENDELSKWWNDT
KHELPDFDKFNYTVPILDIDSEIDRIQGVIQGLNDSLIDLEKLSILKTYIKWPWYVWLAIAFATIIFILILGWVFFMTGC
CGCCCGCFGIMPLMSKCGKKSSYYTTFDNDVVTEQYRPKKSV

| | |
|---|---|
| 1-18 | Signal peptide (SP) |
| 19-532 | S1 |
| 533-537 | Furin cleavage site (FCS) |
| 538-1162 | S2 |
| 1092-1140 | Transmembrane domain (TMD) |
| 1141-1162 | C-terminal domain (CTD) |

IBV-Ma5-Spike-ΔFCS-VSV [SEQ ID NO: 3]
atgctggtgaccccactgctgctggtgacactgctgtgcgcactgtgctccgccgctctgtacgatagctccagctacgt
gtactactaccagagcgcattccggccccctgatggatggcacctgcacggcggagcctacgctgtggtgaacatctcca
gcgagagcaacaatgctggctccagctccggatgcacagtggggatcattcacgggggcagagtggtgaatgcaagctcc
attgcaatgactgcccccagctccggaatggcatggagctccagccagttttgcaccgcctactgcaactttagcgatac
cacagtgttcgtgacacactgctacaagcacggagggtgcccaatcactggaatgctgcagcagcacagcattagggtgt
ccgcaatgaaaaacgggcagctgttctacaatctgacagtgagcgtggccaagtaccccacttttaaatccttccagtgc
gtgaacaatctgaccagcgtgtacctgaacggcgatctggtgtacaccagcaatgctactaccgacgtgacatccgcagg
agtgtactttaaggccggcggacctatcacatacaaagtgatgcgggaagtgagagcactggcctacttcgtgaatggca
ctgctcaggatgtgattctgtgcgatggctccccaaggggactgctggcatgccagtacaacaccggcaatttcagcgac
ggattttaccccttcacaaactccagcctggtgaagcagaaatttatcgtgtaccgcgagaacagcgtgaatacaacttt
cacactgcacaacttcacttttcacaatgaaaccggagccaaccccaatcctagcggggtgcagaacattcagacatacc
agacccagacagctcagagcgggtactacaacttcaatttttccttcctgtccagctttgtgtacaaggagagcaacttc
atgtacggcagctaccacccatcctgcaattttcggctggaaacaatcaacaatgggctgtggttcaactccctgagcgt
gtccattgcttacggccctctgcagggcggctgcaaacagagcgtgttttccggaagagccacctgctgctacgcttact
cctacggagggccactgctgtgcaagggggtgtacagcggcgagctggatcacaatttcgaatgcggactgctggtgtac
gtgaccaaaagcggcggcagcagaatccagactgccaccgagccacccgtgatcacacagcacaactacaacaatattac
actgaacacttgcgtggactacaatatctacgggagaactggccagggattcattaccaacgtgacagatagcgctgtgt
cctacaattacctggctgacgcaggcctggcaatcctggataccagcggcagcatcgacatttttgtggtgcagtccgag
tacggcctgaactactacaaagtgaatccctgcgaagatgtgaaccagcagttcgttgtgagcgggggcaaactggtggg
aatcctgacaagccggaatgagactgggtcccagctgctggaaaaccagttctacatcaagatcactaacggaaccgccg
ccttcgccgccagcatcacagagtccgtggaaaactgcccttacgtgtcctacgggaagttttgcattaaaccagacggc
agcatcgccactattgtgcccaagcagctggagcagtttgtggctcctctgctgaacgtgaccgaaaatgtgctgatccc
aaacagcttcaatctgacagtgactgatgagtacattcagaccaggatggacaaagtgcagatcaactgcctgcagtaca
tttgcgggaacagcctggaatgccgcaatctgttccagcagtacggccctgtgtgcgataacatcctgagcgtggtgaac
agcgtgggccagaaggaggacatggaactgctgaacttttactccagcactaaacccgccggcttcaacacccctgtgct
gagcaatgtgtccaccggagagtttaatatctccctgttcctgaccacaccatccagccccagaaggcgcagctttattg
aggatctgctgttcacaagcgtggaatccgtggggctgcccactgatgacgcttacaagaactgcaccgcaggccctctg
ggattcctgaaagacctggcctgcgctcgcgagtacaatggcctgctggtgctgcctccaatcattacagctgaaatgca
gatcctgtacacttccagcctggtggctagcatggcatttggagggatcactgcagccggggcaattcccttcgccaccc
agctgcaggcaaggatcaaccacctgggcattacacagtccctgctgctgaagaaccaggagaaaatcgctgccagcttc
aataaggctattgggcacatgcaggaaggcttccgcagcacttccctggcactgcagcagatccaggatgtggtgaacaa
gcagtccgccattctgaccgagacaatggctagcctgaacaaaaattttggcgccatctccagcgtgatccaggaaattt
accagcagctggatgctatccaggcaaacgcccaggtggacaggctgattacaggacgcctgtccagcctgagcgtgctg
gcttccgcaaagcaggcagagtacatccgggtgtcccagcagagagagctggccacacagaagatcaacgaatgcgtgaa
aagccagtccattcggtacagcttctgcgggaatggcagacacgtgctgactatccctcagaacgccccaaatggcatcg
tgtttattcacttcagctacacccccgactcctttgtgaacgtgacagctatcgtgggattctgcgtgaagccagccaat
gcttcccagtacgctattgtgcctgcaaacggaagagggatctttattcaagtgaatggaagctactacatcactgcaag
ggatatgtacatgcctcgcgccatcaccgctggggacattgtgactctgaccagctgccaggccaactacgtgtccgtga
ataaaaccgtgatcactaccttcgtggataacgatgactttgatttcaatgacgagctgagcaagtggtggaacgacaca
aaacacgaactgcctgattttgacaagttcaattacactgtgccaatcctggatattgacagcgagatcgataggattca
gggagtgatccaggggctgaacgatagcctgattgacctggaaaaactgtccatcctgaagacatacattaaatcctcca
tcgcttccttcttcttcatcatcggcctgatcatcggactgtttctggtgctgagggtgggcatctacctgtgcatcaag
ctgaagcacactaagaagaggcagatctacaccgacatcgagatgaacaggctgggcaagtga
IBV-Ma5-S-ΔFCS-VSV [SEQ ID NO: 4]
MLVTPLLLVTLLCALCSAALYDSSSYVYYYQSAFRPPDGWHLHGGAYAVVNISSESNNAGSSSGCTVGIIHGGRVVNASS
IAMTAPSSGMAWSSSQFCTAYCNFSDTTVFVTHCYKHGGCPITGMLQQHSIRVSAMKNGQLFYNLTVSVAKYPTFKSFQC
VNNLTSVYLNGDLVYTSNATTDVTSAGVYFKAGGPITYKVMREVRALAYFVNGTAQDVILCDGSPRGLLACQYNTGNFSD
GFYPFTNSSLVKQKFIVYRENSVNTTFTLHNFTFHNETGANPNPSGVQNIQTYQTQTAQSGYYNENFSFLSSFVYKESNE
MYGSYHPSCNFRLETINNGLWENSLSVSIAYGPLQGGCKQSVFSGRATCCYAYSYGGPLLCKGVYSGELDHNFECGLLVY
VTKSGGSRIQTATEPPVITQHNYNNITLNTCVDYNIYGRTGQGFITNVTDSAVSYNYLADAGLAILDTSGSIDIFVVQSE
YGLNYYKVNPCEDVNQQFVVSGGKLVGILTSRNETGSQLLENQFYIKITNGTAAFAASITESVENCPYVSYGKFCIKPDG
SIATIVPKQLEQFVAPLLNVTENVLIPNSFNLTVTDEYIQTRMDKVQINCLQYICGNSLECRNLFQQYGPVCDNILSVVN
SVGQKEDMELLNFYSSTKPAGENTPVLSNVSTGEFNISLFLTTPSSPRRRSFIEDLLFTSVESVGLPTDDAYKNCTAGPL
GFLKDLACAREYNGLLVLPPIITAEMQILYTSSLVASMAFGGITAAGAIPFATQLQARINHLGITQSLLLKNQEKIAASF
NKAIGHMQEGFRSTSLALQQIQDVVNKQSAILTETMASLNKNFGAISSVIQEIYQQLDAIQANAQVDRLITGRLSSLSVL
ASAKQAEYIRVSQQRELATQKINECVKSQSIRYSFCGNGRHVLTIPQNAPNGIVFIHFSYTPDSFVNVTAIVGFCVKPAN
ASQYAIVPANGRGIFIQVNGSYYITARDMYMPRAITAGDIVTLTSCQANYVSVNKTVITTFVDNDDFDENDELSKWWNDT
KHELPDFDKFNYTVPILDIDSEIDRIQGVIQGLNDSLIDLEKLSILKTYIKSSIASFFFIIGLIIGLFLVLRVGIYLCIK
LKHTKKRQIYTDIEMNRLGK

| | |
|---|---|
| 1-18 | Signal peptide (SP) |
| 19-532 | S1 |
| 533-537 | Mutated FCS RRFRR -> AAFAA |
| 538-1091 | S2 |
| 1092-1116 | VSV Transmembrane domain (TMD) |
| 1117-1140 | VSV C-terminal domain (CTD) |

IBV-Ma5-Spike-ΔFCS-2P-VSV [SEQ ID NO: 5]
atgctggtgaccccactgctgctggtgacactgctgtgcgcactgtgctccgccgctctgtacgatagctccagctacgt
gtactactaccagagcgcattccggccccctgatggatggcacctgcacggcggagcctacgctgtggtgaacatctcca
gcgagagcaacaatgctggctccagctccggatgcacagtggggatcattcacgggggcagagtggtgaatgcaagctcc
attgcaatgactgcccccagctccggaatggcatggagctccagccagttttgcaccgcctactgcaactttagcgatac
cacagtgttcgtgacacactgctacaagcacggagggtgcccaatcactggaatgctgcagcagcacagcattagggtgt
ccgcaatgaaaaacgggcagctgttctacaatctgacagtgagcgtggccaagtaccccacttttaaatccttccagtgc
gtgaacaatctgaccagcgtgtacctgaacggcgatctggtgtacaccagcaatgctactaccgacgtgacatccgcagg
agtgtactttaaggccggcggacctatcacatacaaagtgatgcgggaagtgagagcactggcctacttcgtgaatggca
ctgctcaggatgtgattctgtgcgatggctccccaaggggactgctggcatgccagtacaacaccggcaatttcagcgac
ggattttaccccttcacaaactccagcctggtgaagcagaaatttatcgtgtaccgcgagaacagcgtgaatacaacttt
cacactgcacaacttcacttttcacaatgaaaccggagccaaccccaatcctagcggggtgcagaacattcagacatacc
agacccagacagctcagagcgggtactacaacttcaatttttccttcctgtccagctttgtgtacaaggagagcaacttc
atgtacggcagctaccacccatcctgcaattttcggctggaaacaatcaacaatgggctgtggttcaactccctgagcgt
gtccattgcttacggccctctgcagggcggctgcaaacagagcgtgttttccggaagagccacctgctgctacgcttact
cctacggagggccactgctgtgcaagggggtgtacagcggcgagctggatcacaatttcgaatgcggactgctggtgtac
gtgaccaaaagcggcggcagcagaatccagactgccaccgagccacccgtgatcacacagcacaactacaacaatattac
actgaacacttgcgtggactacaatatctacgggagaactggccagggattcattaccaacgtgacagatagcgctgtgt
cctacaattacctggctgacgcaggcctggcaatcctggataccagcggcagcatcgacatttttgtggtgcagtccgag
tacggcctgaactactacaaagtgaatccctgcgaagatgtgaaccagcagttcgttgtgagcgggggcaaactggtggg
aatcctgacaagccggaatgagactgggtcccagctgctggaaaaccagttctacatcaagatcactaacggaaccgccg
ccttcgccgccagcatcacagagtccgtggaaaactgcccttacgtgtcctacgggaagttttgcattaaaccagacggc
agcatcgccactattgtgcccaagcagctggagcagtttgtggctcctctgctgaacgtgaccgaaaatgtgctgatccc
aaacagcttcaatctgacagtgactgatgagtacattcagaccaggatggacaaagtgcagatcaactgcctgcagtaca
tttgcgggaacagcctggaatgccgcaatctgttccagcagtacggccctgtgtgcgataacatcctgagcgtggtgaac
agcgtgggccagaaggaggacatggaactgctgaacttttactccagcactaaacccgccggcttcaacacccctgtgct
gagcaatgtgtccaccggagagtttaatatctccctgttcctgaccacaccatccagccccagaaggcgcagctttattg
aggatctgctgttcacaagcgtggaatccgtggggctgcccactgatgacgcttacaagaactgcaccgcaggccctctg
ggattcctgaaagacctggcctgcgctcgcgagtacaatggcctgctggtgctgcctccaatcattacagctgaaatgca
gatcctgtacacttccagcctggtggctagcatggcatttggagggatcactgcagccggggcaattcccttcgccaccc
agctgcaggcaaggatcaaccacctgggcattacacagtccctgctgctgaagaaccaggagaaaatcgctgccagcttc
aataaggctattgggcacatgcaggaaggcttccgcagcacttccctggcactgcagcagatccaggatgtggtgaacaa
gcagtccgccattctgaccgagacaatggctagcctgaacaaaaattttggcgccatctccagcgtgatccaggaaattt
accagcagctggatcccccccaggcaaacgcccaggtggacaggctgattacaggacgcctgtccagcctgagcgtgctg
gcttccgcaaagcaggcagagtacatccgggtgtcccagcagagagagctggccacacagaagatcaacgaatgcgtgaa
aagccagtccattcggtacagcttctgcgggaatggcagacacgtgctgactatccctcagaacgccccaaatggcatcg
tgtttattcacttcagctacacccccgactcctttgtgaacgtgacagctatcgtgggattctgcgtgaagccagccaat
gcttcccagtacgctattgtgcctgcaaacggaagagggatctttattcaagtgaatggaagctactacatcactgcaag
ggatatgtacatgcctcgcgccatcaccgctggggacattgtgactctgaccagctgccaggccaactacgtgtccgtga
ataaaaccgtgatcactaccttcgtggataacgatgactttgatttcaatgacgagctgagcaagtggtggaacgacaca
aaacacgaactgcctgattttgacaagttcaattacactgtgccaatcctggatattgacagcgagatcgataggattca
gggagtgatccaggggctgaacgatagcctgattgacctggaaaaactgtccatcctgaagacatacattaaatcctcca
tcgcttccttcttcttcatcatcggcctgatcatcggactgtttctggtgctgagggtgggcatctacctgtgcatcaag
ctgaagcacactaagaagaggcagatctacaccgacatcgagatgaacaggctgggcaagtga
IBV-Ma5-S-ΔFCS-2P-VSV [SEQ ID NO: 6]
MLVTPLLLVTLLCALCSAALYDSSSYVYYYQSAFRPPDGWHLHGGAYAVVNISSESNNAGSSSGCTVGIIHGGRVVNASS
IAMTAPSSGMAWSSSQFCTAYCNFSDTTVFVTHCYKHGGCPITGMLQQHSIRVSAMKNGQLFYNLTVSVAKYPTFKSFQC
VNNLTSVYLNGDLVYTSNATTDVTSAGVYFKAGGPITYKVMREVRALAYFVNGTAQDVILCDGSPRGLLACQYNTGNFSD
GFYPFTNSSLVKQKFIVYRENSVNTTFTLHNFTFHNETGANPNPSGVQNIQTYQTQTAQSGYYNENFSFLSSFVYKESNF
MYGSYHPSCNFRLETINNGLWENSLSVSIAYGPLQGGCKQSVFSGRATCCYAYSYGGPLLCKGVYSGELDHNFECGLLVY
VTKSGGSRIQTATEPPVITQHNYNNITLNTCVDYNIYGRTGQGFITNVTDSAVSYNYLADAGLAILDTSGSIDIFVVQSE
YGLNYYKVNPCEDVNQQFVVSGGKLVGILTSRNETGSQLLENQFYIKITNGTAAFAASITESVENCPYVSYGKFCIKPDG
SIATIVPKQLEQFVAPLLNVTENVLIPNSFNLTVTDEYIQTRMDKVQINCLQYICGNSLECRNLFQQYGPVCDNILSVVN
SVGQKEDMELLNFYSSTKPAGENTPVLSNVSTGEFNISLFLTTPSSPRRRSFIEDLLFTSVESVGLPTDDAYKNCTAGPL
GFLKDLACAREYNGLLVLPPIITAEMQILYTSSLVASMAFGGITAAGAIPFATQLQARINHLGITQSLLLKNQEKIAASE
NKAIGHMQEGFRSTSLALQQIQDVVNKQSAILTETMASLNKNFGAISSVIQEIYQQLDPPQANAQVDRLITGRLSSLSVL
ASAKQAEYIRVSQQRELATQKINECVKSQSIRYSFCGNGRHVLTIPQNAPNGIVFIHFSYTPDSFVNVTAIVGFCVKPAN
ASQYAIVPANGRGIFIQVNGSYYITARDMYMPRAITAGDIVTLTSCQANYVSVNKTVITTFVDNDDEDENDELSKWWNDT
KHELPDFDKFNYTVPILDIDSEIDRIQGVIQGLNDSLIDLEKLSILKTYIKSSIASFFFIIGLIIGLFLVLRVGIYLCIK
LKHTKKRQIYTDIEMNRLGK

| | |
|---|---|
| 1-18 | Signal peptide (SP) |
| 19-532 | S1 |
| 533-537 | Mutated FCS RRFRR -> AAFAA |
| 538-1091 | S2 |
| 859-860 | A859P + I860P substitutions |
| 1092-1116 | VSV Transmembrane domain (TMD) |
| 1117-1140 | VSV C-terminal domain (CTD) |

SARS-CoV-2-Spike [SEQ ID NO: 7]
atgttcgtgttcctggtgctgctgcccctggtgtccagccagtgcgtgaacctgaccaccaggacccagctgccaccagc
ctacaccaacagcttcaccaggggcgtgtactaccccgacaaagtgttcagatcttccgtgctgcacagcacccaggacc
tgttcctgcccttcttctctaacgtgacctggttccacgccatccacgtgtccggcaccaacggcaccaagaggttcgac
aaccccgtgctgcccttcaacgacggcgtgtacttcgccagcaccgagaagtctaacatcatcagaggctggatcttcgg
caccaccctggactccaaaacccagagcctgctgatcgtgaacaacgccaccaacgtggtcatcaaggtgtgcgagttcc
agttctgtaacgaccccttcctgggcgtgtactaccacaagaacaacaaatcttggatggagtccgagttcagggtgtac
agctctgccaacaactgcaccttcgagtacgtgagccagcccttcctgatggacctggaaggcaagcagggcaacttcaa
aaacctgcgggagttcgtgttcaagaacatcgacggctacttcaagatctacagcaaacacacccccatcaacctggtgc
gcgacctgccacagggcttctctgccctggagccactggtggacctgccaatcggcatcaacatcaccaggttccagacc
ctgctggccctgcacagatcctacctgaccccaggcgactccagctctggatggaccgctggagctgccgcctactacgt
gggctacctgcagccccggaccttcctgctgaaatacaacgagaacggaaccatcaccgacgctgtggactgcgctctgg
acccactgtctgaaaccaagtgtaccctgaaatccttcaccgtggagaagggcatctaccagacctccaacttccgggtg
cagcccaccgaaagcatcgtgcgcttccccaacatcaccaacctgtgccccttcggcgaggtgttcaacgctaccaggtt
cgctagcgtgtacgcttggaaccggaagcgcatcagcaactgcgtggccgactactctgtgctgtacaactccgccagct
tctctaccttcaagtgctacggcgtgtcccccaccaaactgaacgacctgtgcttcaccaacgtgtacgccgacagcttc
gtgatcaggggcgacgaggtgcgccagatcgctccaggacagaccggcaagatcgctgactacaactacaaactgcccga
cgacttcaccggctgcgtgatcgcctggaactctaacaacctggactccaaagtgggcggcaactacaactacctgtaca
ggctgttcagaaagtctaacctgaaacccttcgagcgggacatcagcaccgaaatctaccaggctggatctaccccatgc
aacggagtggagggcttcaactgttacttccccctgcagtcctacggcttccagccaaccaacggagtgggataccagcc
atacagggtggtggtgctgtctttcgaactgctgcacgctccagctaccgtgtgcggacccaagaaatccaccaacctgg
tgaagaacaaatgcgtgaacttcaacttcaacggactgaccggaaccggcgtgctgaccgagagcaacaagaaattcctg
cccttccagcagttcggccgggacatcgctgacaccaccgacgccgtgcgcgacccccagaccctggaaatcctggacat
caccccctgcagcttcggcggcgtgtctgtgatcaccccaggaaccaacacctccaaccaggtggccgtgctgtaccagg
acgtgaactgtaccgaggtgccagtggctatccacgctgaccagctgaccccaacctggagggtgtactctaccggctcc
aacgtgttccagaccagagctggatgcctgatcggagctgagcacgtgaacaactcctacgaatgcgacatccccatcgg
cgccggcatctgtgccagctaccagacccagaccaacagcccaaggagagccaggtctgtggcttcccagagcatcatcg
cctacaccatgtccctgggcgccgaaaacagcgtggcctacagcaacaactctatcgccatccccaccaacttcaccatc
agcgtgaccaccgagatcctgcccgtgtccatgaccaagaccagcgtggactgcaccatgtacatctgtggcgacagcac
cgaatgctctaacctgctgctgcagtacggctccttctgtacccagctgaacagagccctgaccggaatcgctgtggagc
aggacaaaaacacccaggaagtgttcgcccaggtgaagcagatctacaaaaccccccccatcaaggacttcggcggcttc
aacttctcccagatcctgcccgacccctccaagcccagcaaaaggtctttcatcgaggacctgctgttcaacaaggtgac
cctggccgacgccggcttcatcaaacagtacggcgactgcctgggcgacatcgctgctagagacctgatctgtgcccaga
agttcaacggactgaccgtgctgccaccactgctgaccgacgaaatgatcgctcagtacacctctgccctgctggctgga
accatcacctccggatggaccttcggcgctggagccgccctgcagatccccttcgccatgcagatggcctacagattcaa
cggcatcggcgtgacccagaacgtgctgtacgagaaccagaagctgatcgccaaccagttcaacagcgccatcggcaaaa
tccaggactctctgtccagcaccgcttccgccctgggcaaactgcaggacgtggtgaaccagaacgcccaggccctgaac
accctggtgaagcagctgtcttccaacttcggcgccatcagctctgtgctgaacgacatcctgtccaggctggacaaagt
ggaggccgaagtgcagatcgacaggctgatcaccggcagactgcagagcctgcagacctacgtgacccagcagctgatca
gggctgctgaaatcagggcttctgccaacctggctgctaccaagatgtccgagtgcgtgctgggccagagcaagagagtg
gacttctgtggcaaaggctaccacctgatgtccttcccacagagcgccccacacggagtggtgttcctgcacgtgaccta
cgtgcccgcccaggagaagaacttcaccaccgctccagctatctgccacgacggcaaagctcacttcccaagggaaggcg
tgttcgtgtccaacggcacccactggttcgtgacccagcgcaacttctacgagccccagatcatcaccaccgacaacacc
ttcgtgagcggcaactgtgacgtggtcatcggaatcgtgaacaacaccgtgtacgacccactgcagccagagctggactc
tttcaaggaggaactggacaagtacttcaaaaaccacacctccccagacgtggacctgggcgacatctctggcatcaacg
cctccgtggtgaacatccagaaggagatcgacaggctgaacgaagtggccaaaaacctgaacgaaagcctgatcgacctg
caggagctgggcaagtacgaacagtacatcaaatggccctggtacatctggctgggcttcatcgccggcctgatcgccat
cgtgatggtgaccatcatgctgtgctgtatgacctcctgctgtagctgcctgaagggctgctgttcttgtggctcctgct
gtaaattcgacgaggacgactccgaacccgtgctgaagggcgtgaaactgcactacacctga
SARS-CoV-2-Spike [SEQ ID NO: 8]
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLENKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT

| | |
|---|---|
| 1-13 | Signal peptide (SP) |
| 14-681 | S1 |
| 333-527 | Receptor binding domain (RBD) |
| 682-685 | Furin cleavage site (FCS) |
| 686-1211 | S2 |
| 1212-1255 | Transmembrane domain (TMD) |
| 1256-1273 | C-terminal domain (CTD) |

SARS-CoV-2-Spike-ΔFCS-VSV [SEQ ID NO: 9]
atgttcgtgttcctggtgctgctgcccctggtgtccagccagtgcgtgaacctgaccaccagaacccagctgccaccagc
ctacaccaacagcttcacccggggcgtgtactaccccgacaaagtgttccgctcttccgtgctgcactctacccaggacc
tgttcctgcccttcttctccaacgtgacctggttccacgccatccacgtgtccggcaccaacggcaccaagaggttcgac
aaccccgtgctgcccttcaacgacggcgtgtacttcgcctctaccgagaagtccaacatcatcagaggctggatcttcgg
caccaccctggacagcaaaacccagtctctgctgatcgtgaacaacgccaccaacgtggtcatcaaggtgtgcgagttcc
agttctgtaacgaccccttcctgggcgtgtactaccacaagaacaacaaatcctggatggagagcgagttcagggtgtac
agctctgccaacaactgtaccttcgagtacgtgagccagcccttcctgatggacctggaaggcaagcagggcaacttcaa
aaacctgcgggagttcgtgttcaagaacatcgacggctacttcaagatctactctaaacacacccccatcaacctggtgc
gcgacctgccacagggcttctccgccctggagccactggtggacctgcccatcggcatcaacatcaccaggttccagacc
ctgctggccctgcaccgctcctacctgaccccaggcgactccagctctggatggaccgctggagctgccgcctactacgt
gggctacctgcagcccaggaccttcctgctgaaatacaacgaaaacggaaccatcaccgacgctgtggactgcgctctgg
acccactgtccgaaaccaagtgtaccctgaaaagcttcaccgtggagaagggcatctaccagaccagcaacttcagggtg
cagcccaccgaatctatcgtgagattccccaacatcaccaacctgtgccccttcggcgaggtgttcaacgccaccagatt
cgccagcgtgtacgcctggaacaggaagagaatctctaactgcgtggccgactactccgtgctgtacaactctgcctcct
tcagcaccttcaagtgctacggcgtgagccccaccaaactgaacgacctgtgcttcaccaacgtgtacgccgactctttc
gtgatcaggggcgacgaggtgagacagatcgctccaggacagaccggcaagatcgctgactacaactacaaactgcccga
cgacttcaccggctgcgtgatcgcctggaactccaacaacctggacagcaaagtgggcggcaactacaactacctgtacc
ggctgttccgcaagagcaacctgaaacccttcgagcgggacatctctaccgaaatctaccaggctggatccaccccatgc
aacggagtggagggcttcaactgttacttccccctgcagtcctacggcttccagccaaccaacggagtgggataccagcc
atacagggtggtggtgctgtccttcgaactgctgcacgctccagctaccgtgtgcggacccaagaaaagcaccaacctgg
tgaagaacaaatgcgtgaacttcaacttcaacggactgaccggaaccggcgtgctgaccgagagcaacaagaaattcctg
cccttccagcagttcggaagggacatcgctgacaccaccgacgccgtgagagacccacagaccctggaaatcctggacat
caccccctgctctttcggcggcgtgtccgtgatcaccccaggaaccaacacctccaaccaggtggccgtgctgtaccagg
acgtgaactgtaccgaggtgccagtggctatccacgctgaccagctgaccccaacctggagggtgtacagcaccggctct
aacgtgttccagaccagagctggatgcctgatcggagctgagcacgtgaacaacagctacgaatgcgacatccccatcgg
cgccggcatctgtgcctcttaccagacccagaccaactctccagctgccgcccggtccgtggcttctcagtccatcatcg
cctacaccatgagcctgggcgccgaaaactctgtggcctactccaacaacagcatcgccatccccaccaacttcaccatc
agcgtgaccaccgagatcctgcccgtgagcatgaccaagacctctgtggactgcaccatgtacatctgtggcgactctac
cgaatgctccaacctgctgctgcagtacggctccttctgtacccagctgaaccgcgccctgaccggaatcgctgtggagc
aggacaaaaacacccaggaagtgttcgcccaggtgaagcagatctacaaaaccccccccatcaaggacttcggcggcttc
aacttctcccagatcctgcccgacccctctaagccctccaaaaggagcttcatcgaggacctgctgttcaacaaggtgac
cctggccgacgccggcttcatcaaacagtacggcgactgcctgggcgacatcgctgctagagacctgatctgtgcccaga
agttcaacggactgaccgtgctgccaccactgctgaccgacgaaatgatcgctcagtacacctccgccctgctggctgga
accatcaccagcggatggaccttcggcgctggagccgccctgcagatccccttcgccatgcagatggcctacaggttcaa
cggcatcggcgtgacccagaacgtgctgtacgagaaccagaagctgatcgccaaccagttcaacagcgccatcggcaaaa
tccaggactccctgtccagcaccgctagcgccctgggcaaactgcaggacgtggtgaaccagaacgcccaggccctgaac
accctggtgaagcagctgtcttccaacttcggcgccatcagctctgtgctgaacgacatcctgtcccggctggacaaagt
ggaggccgaagtgcagatcgacaggctgatcaccggccgcctgcagtctctgcagacctacgtgacccagcagctgatca
gggccgccgaaatcagagcctccgccaacctggccgccaccaagatgagcgagtgcgtgctgggccagtctaagcgcgtg
gacttctgtggcaaaggctaccacctgatgagcttcccacagtctgccccacacggagtggtgttcctgcacgtgaccta
cgtgcccgcccaggagaagaacttcaccaccgctccagctatctgccacgacggcaaagctcacttcccaagggaaggcg
tgttcgtgagcaacggcacccactggttcgtgacccagcgcaacttctacgagccccagatcatcaccaccgacaacacc
ttcgtgtccggcaactgtgacgtggtcatcggaatcgtgaacaacaccgtgtacgacccactgcagccagagctggactc
cttcaaggaggaactggacaagtacttcaaaaaccacaccagcccagacgtggacctgggcgacatctccggcatcaacg
ccagcgtggtgaacatccagaaggagatcgacaggctgaacgaagtggccaaaaacctgaacgaaagcctgatcgacctg
caggagctgggcaagtacgaacagtacatcaaatccagcatcgcctccttcttcttcatcatcggcctgatcatcggcct
gttcctggtgctgagagtgggcatctacctgtgcatcaagctgaaacacaccaagaaacggcagatctacaccgacatcg
agatgaaccgcctgggcaagtga
SARS-COV-2-Spike-ΔFCS-VSV [SEQ ID NO: 10]
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGESALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPAAARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKSSIASFFFIIGLIIGLFLVLRVGIYLCIKLKHTKKRQIYTDIEMNRLGK

| | |
|---|---|
| 1-13 | Signal peptide |
| 14-681 | S1 |
| 333-527 | Receptor binding domain (RBD) |
| 682-685 | Mutated FCS RRAR -> AAAR |
| 686-1211 | S2 |
| 1212-1236 | VSV Transmembrane domain (TMD) |
| 1237-1260 | VSV C-terminal domain (CTD) |

SARS-CoV-2-Spike-ΔFCS-2P-VSV [SEQ ID NO: 11]
atgttcgtgttcctggtgctgctgcccctggtgtccagccagtgcgtgaacctgaccaccagaacccagctgccaccagc
ctacaccaacagcttcacccggggcgtgtactaccccgacaaagtgttccgctcttccgtgctgcactctacccaggacc
tgttcctgcccttcttctccaacgtgacctggttccacgccatccacgtgtccggcaccaacggcaccaagaggttcgac
aaccccgtgctgcccttcaacgacggcgtgtacttcgcctctaccgagaagtccaacatcatcagaggctggatcttcgg
caccaccctggacagcaaaacccagtctctgctgatcgtgaacaacgccaccaacgtggtcatcaaggtgtgcgagttcc
agttctgtaacgaccccttcctgggcgtgtactaccacaagaacaacaaatcctggatggagagcgagttcagggtgtac
agctctgccaacaactgtaccttcgagtacgtgagccagcccttcctgatggacctggaaggcaagcagggcaacttcaa
aaacctgcgggagttcgtgttcaagaacatcgacggctacttcaagatctactctaaacacacccccatcaacctggtgc
gcgacctgccacagggcttctccgccctggagccactggtggacctgcccatcggcatcaacatcaccaggttccagacc
ctgctggccctgcaccgctcctacctgaccccaggcgactccagctctggatggaccgctggagctgccgcctactacgt
gggctacctgcagcccaggaccttcctgctgaaatacaacgaaaacggaaccatcaccgacgctgtggactgcgctctgg
acccactgtccgaaaccaagtgtaccctgaaaagcttcaccgtggagaagggcatctaccagaccagcaacttcagggtg
cagcccaccgaatctatcgtgagattccccaacatcaccaacctgtgccccttcggcgaggtgttcaacgccaccagatt
cgccagcgtgtacgcctggaacaggaaaagaatctctaactgcgtggccgactactccgtgctgtacaactctgcctcct
tcagcaccttcaagtgctacggcgtgagccccaccaaactgaacgacctgtgcttcaccaacgtgtacgccgactctttc
gtgatcaggggcgacgaggtgagacagatcgctccaggacagaccggcaagatcgctgactacaactacaaactgcccga
cgacttcaccggctgcgtgatcgcctggaactccaacaacctggacagcaaagtgggcggcaactacaactacctgtacc
ggctgttccgcaagagcaacctgaaacccttcgagcgggacatctctaccgaaatctaccaggctggatccaccccatgc
aacggagtggagggcttcaactgttacttccccctgcagtcctacggcttccagccaaccaacggagtgggataccagcc
atacagggtggtggtgctgtccttcgaactgctgcacgctccagctaccgtgtgcggacccaagaaaagcaccaacctgg
tgaagaacaaatgcgtgaacttcaacttcaacggactgaccggaaccggcgtgctgaccgagagcaacaagaaattcctg
cccttccagcagttcggaagggacatcgctgacaccaccgacgccgtgagagacccacagaccctggaaatcctggacat
caccccctgctctttcggcggcgtgtccgtgatcaccccaggaaccaacacctccaaccaggtggccgtgctgtaccagg
acgtgaactgtaccgaggtgccagtggctatccacgctgaccagctgaccccaacctggagggtgtacagcaccggctct
aacgtgttccagaccagagctggatgcctgatcggagctgagcacgtgaacaacagctacgaatgcgacatccccatcgg
cgccggcatctgtgcctcttaccagacccagaccaactctccagctgccgcccggtccgtggcttctcagtccatcatcg
cctacaccatgagcctgggcgccgaaaactctgtggcctactccaacaacagcatcgccatccccaccaacttcaccatc
agcgtgaccaccgagatcctgcccgtgagcatgaccaagacctctgtggactgcaccatgtacatctgtggcgactctac
cgaatgctccaacctgctgctgcagtacggctccttctgtacccagctgaaccgcgccctgaccggaatcgctgtggagc
aggacaaaaacacccaggaagtgttcgcccaggtgaagcagatctacaaaaccccccccatcaaggacttcggcggcttc
aacttctcccagatcctgcccgacccctctaagccctccaaaaggagcttcatcgaggacctgctgttcaacaaggtgac
cctggccgacgccggcttcatcaaacagtacggcgactgcctgggcgacatcgctgctagagacctgatctgtgcccaga
agttcaacggactgaccgtgctgccaccactgctgaccgacgaaatgatcgctcagtacacctccgccctgctggctgga
accatcaccagcggatggaccttcggcgctggagccgccctgcagatccccttcgccatgcagatggcctacaggttcaa
cggcatcggcgtgacccagaacgtgctgtacgagaaccagaagctgatcgccaaccagttcaacagcgccatcggcaaaa
tccaggactccctgtccagcaccgctagcgccctgggcaaactgcaggacgtggtgaaccagaacgcccaggccctgaac
accctggtgaagcagctgtcttccaacttcggcgccatcagctctgtgctgaacgacatcctgtccaggctggacccacc
agaggctgaagtgcagatcgacaggctgatcaccggccgcctgcagtctctgcagacctacgtgacccagcagctgatca
gggccgccgaaatcagagcctccgccaacctggccgccaccaagatgagcgagtgcgtgctgggccagtctaagcgcgtg
gacttctgtggcaaaggctaccacctgatgagcttcccacagtctgccccacacggagtggtgttcctgcacgtgaccta
cgtgcccgcccaggagaagaacttcaccaccgctccagctatctgccacgacggcaaagctcacttcccaagggaaggcg
tgttcgtgagcaacggcacccactggttcgtgacccagcgcaacttctacgagccccagatcatcaccaccgacaacacc
ttcgtgtccggcaactgtgacgtggtcatcggaatcgtgaacaacaccgtgtacgacccactgcagccagagctggactc
cttcaaggaggaactggacaagtacttcaaaaaccacaccagcccagacgtggacctgggcgacatctccggcatcaacg
ccagcgtggtgaacatccagaaggagatcgacaggctgaacgaagtggccaaaaacctgaacgaaagcctgatcgacctg
caggagctgggcaagtacgaacagtacatcaaatccagcatcgcctccttcttcttcatcatcggcctgatcatcggcct
gttcctggtgctgagagtgggcatctacctgtgcatcaagctgaaacacaccaagaaacggcagatctacaccgacatcg
agatgaaccgcctgggcaagtga
SARS-CoV-2-Spike-ΔFCS-2P-VSV [SEQ ID NO: 12]
MFVFLVLLPLVSSQCVNLTTRTQLPPAYTNSFTRGVYYPDKVFRSSVLHSTQDLFLPFFSNVTWFHAIHVSGTNGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPAAARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLENKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLSSTASALGKLQDVVNQNAQALN
TLVKQLSSNFGAISSVLNDILSRLDPPEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKSSIASFFFIIGLIIGLFLVLRVGIYLCIKLKHTKKRQIYTDIEMNRLGK

| | |
|---|---|
| 1-13 | Signal peptide (SP) |
| 14-681 | S1 |
| 333-527 | Receptor binding domain (RBD) |
| 682-685 | Mutated FCS RRAR -> AAAR |
| 686-1211 | S2 |
| 986-987 | K986P + V987P substitutions |
| 1212-1236 | VSV Transmembrane domain (TMD) |
| 1237-1260 | VSV C-terminal domain (CTD) |

SEQUENCE LISTING TABLE

| SEQ | | SARS- | | | |
|---|---|---|---|---|---|
| ID | IBV | CoV2 | NA | AA | Description |
| 1 | √ | | √ | | WT |
| 2 | √ | | | √ | WT |
| 3 | √ | | √ | | ΔFCS-VSV |
| 4 | √ | | | √ | ΔFCS-VSV |
| 5 | √ | | √ | | ΔFCS-2P-VSV |
| 6 | √ | | | √ | ΔFCS-2P-VSV |
| 7 | | √ | √ | | WT |
| 8 | | √ | | √ | WT |
| 9 | | √ | √ | | ΔFCS-VSV |
| 10 | | √ | | √ | ΔFCS-VSV |
| 11 | | √ | √ | | ΔFCS-2P-VSV |
| 12 | | √ | | √ | ΔFCS-2P-VSV |
| 13 | | √ | | √ | Cleavage site |
| 14 | | | | √ | Cleavage site |
| 15 | | | | √ | Cleavage site |

### Example 2

### Incorporation of the Coding Sequences for SARS-CoV-2 and IBV Spike Protein into the VEEV RNA Replicon Particles

Alphavirus RNA Replicon Construction

Vaccines were prepared comprising an alphavirus RNA replicon particle encoding codon-optimized SARS-CoV-2 Spike Protein (SARS-CoV-2-S-wt), corresponding SARS-CoV-2 Spike chimeric spike proteins (SARS-CoV-2-S-AFCS, SARS-CoV-2-S-AFCS-2P, SARS-CoV-2-S-ΔFCS-ΔCTD, SARS-CoV-2-S-ΔFCS-VSV, SARS-CoV-2-S-ΔFCS-2P-VSV), and codon optimized IBV Spike (IBV-S-wt), and corresponding IBV Spike chimeric spike proteins (IBV-S-2P-ΔCTD, IBV-S-2P-Y1144A, IBV-S-2P-VSV).

Generation of SARS-CoV-2 Spike Protein Gene RPs.

The VEEV replicon vector for use to express the SARS-CoV-2 Spike gene is constructed as previously described [see, U.S. Pat. No. 9,441,247 B2; the contents of which are hereby incorporated herein by reference], with the following modifications. The VEEV TC-83-derived replicon vector “pVEK” [disclosed and described in U.S. Pat. No. 9,441,247 B2] is digested with restriction enzymes AscI and PacI to create the vector “pVHV”. The spike protein gene sequence from SARS-CoV-2, strain 2019-nCoV/USA-WI1/2020 (GenBank accession MT039887), was codon-optimized towards the codon use table of cat, and synthesized with flanking AscI and PacI sites. The synthetic gene and pVHV vector are each digested with AscI and PacI enzymes and ligated to create vector “pVHV-SARS-CoV-2-Spike”. Plasmid batches are sequenced to confirm the correct vector and insert identities.

Generation of SARS-CoV-2 Spike Protein Gene RNA RPs.

The VEEV replicon vector for use to express the SARS-CoV-2 Spike (SARS-CoV-2-S-wt) gene and the corresponding SARS-CoV-2 Spike chimeric spike proteins (SARS-CoV-2-S-AFCS, SARS-CoV-2-S-ΔFCS-2P, SARS-CoV-2-S-ΔFCS-ΔCTD, SARS-CoV-2-S-ΔFCS-VSV, SARS-CoV-2-S-ΔFCS-2P-VSV) were constructed as previously described [see, U.S. Pat. No. 9,441,247 B2; the contents of which are hereby incorporated herein by reference], with the following modifications. The VEEV TC-83-derived replicon vector “pVEK” [disclosed and described in U.S. Pat. No. 9,441,247 B2] is digested with restriction enzymes AscI and PacI to create the vector “pVHV”. The spike protein gene sequence from SARS-CoV-2, strain 2019-nCoV/USA-WI1/2020 (GenBank accession MT039887), and corresponding SARS-CoV-2 Spike chimeric spike proteins) were codon-optimized and synthesized with flanking AscI and PacI sites. The synthetic gene and pVHV vector are each digested with AscI and PacI enzymes and ligated to create vector “pVHV-SARS-CoV-2-Spike”. Plasmid batches are sequenced to confirm the correct vector and insert identities.

Generation of IBV Spike Protein Gene RNA RPs.

Similar to the spike protein gene sequence from SARS-CoV-2, the spike protein gene sequence from IBV, strain Ma5 (GenBank accession KY626045), was codon-optimized and synthesized with flanking AscI and PacI sites. The VEEV replicon vector for use to express the IBV Spike (IBV-S-wt) gene and the corresponding IBV Spike chimeric spike proteins (IBV-S-2P-CTD, IBV-S-2P-Y1144A, IBV-S-2P-VSV, IBV-S-AFCS, IBV-S-ΔFCS-2P, IBV-S-ΔFCS-ΔCTD, IBV-S-ΔFCS-VSV, IBV-S-ΔFCS-2P-VSV) were constructed as described above for the above SARS-CoV-2 Spike protein. Accordingly, similar to the spike protein gene sequence from SARS-CoV-2, the spike protein gene sequence from IBV, strain Ma5 (GenBank accession KY626045), was codon-optimized and is synthesized with flanking AscI and PacI sites. The synthetic gene and pVHV vector are each digested with AscI and PacI enzymes and ligated to create vector “pVHV-IBV-Ma5-Spike”. Plasmid batches are sequenced to confirm the correct vector and insert identities.

Production of VEEV TC-83 RNA RPs is conducted according to methods previously described [U.S. Pat. No. 9,441,247 B2 and U.S. Pat. No. 8,460,913 B2; the contents of which are hereby incorporated herein by reference]. Briefly, pVHV-Spike replicon vector DNA and helper DNA plasmids are linearized with NotI restriction enzyme prior to in vitro transcription using MegaScript T7 RNA polymerase and cap analog. Importantly, the helper RNAs that are used in the production lack the VEEV subgenomic promoter sequence, as previously described [Kamrud et al., J Gen Virol. 91 (Pt 7):1723-1727 (2010)]. Purified RNAs for the replicon and helper components are combined and mixed with a suspension of Vero cells, electroporated in 4 mm cuvettes, and returned to serum-free culture media. Following overnight incubation, alphavirus RNA replicon particles are purified from the cells and media by passing the suspension through a depth filter, washing with phosphate buffered saline containing 5% sucrose (w/v), and finally eluting the retained RP with 400 mM NaCl+5% sucrose (w/v) buffer. Eluted RP are passed through a 0.22 micron membrane filter, and dispensed into aliquots for storage. Titer of functional RP is determined by immunofluorescence assay on infected Vero cell monolayers. The resulting propagation-defective alphavirus RNA replicon particle encoding codon optimized SARS-CoV-2 spike protein can then be placed into a non-adjuvanted or adjuvanted vaccine formulation and administered to the animal subject.

### Example 3

### Expression of IBV Spike Antigens in Cultured Cells Using IFA

To investigate the expression of IBV Spike antigens in host cells, a series of experiments were performed using different forms for the delivery of the polypeptide according to the invention to host cells. Different staining techniques were applied to visualize the type and the location of those expressions.

IBV Spike Antigen From Plasmid DNA in Vero Cells

To determine if inactivation of the furin cleavage site (ΔFCS), removal of C-terminal domain (ΔCTD) or replacement of the spike protein TMD and CTD by the surface glycoprotein TMD and CTD of VSV, the proline mutation (2P), the addition of a trimerization domain (3M), or the mutation of the ER-retention signal (Y¹¹⁴⁴A) has any effect on the expression levels of the IBV Spike antigens, Vero cells were transfected with the pCAGGS expression plasmids that drive the production of the IBV Spike antigens and used for immunofluorescence assay (IFA).

### Materials & Methods

Vero cells were cultured in DMEM supplemented with 10% FCS, L-Glutamine, and 1% non-essential amino-acids. Cells for transfection were seeded at a density of 25.000 cells/cm² in 24-well clusters in 0.5 ml culture medium and incubated at 37° C., 5% CO₂. Next day, semi-confluent monolayers of Vero cells were transfected with 500 ng pCAGGS plasmid DNA using Lipofectamine3000™ (ThermoFisher) in 50 μl transfection mix according to manufacturer's instructions per well. Twenty-four hours post transfection/infection, cells were washed once with ˜1 ml phosphate-buffered saline (PBS) per well and fixed using 0.5 ml 96% ethanol per well for 30 minutes at −20° C. Cells were washed three times using ˜1 ml wash buffer (PBS+0.15% polysorbate 20) per well and Spike antigens were visualized using either the INT-M41-01-03 mouse monoclonal antibody or a chicken polyclonal antibody serum from Charles River in 0.25 ml IBEIA buffer (PBS+0.05% polysorbate 20+0.1% BSA) for 1 hour at room temperature. Bound antibodies were stained using secondary Goat anti-mouse IgG Alexa488 or Goat anti-chicken IgG Alexa568 antibodies (ThermoFisher) in 0.25 ml IBEIA buffer for 1 hour at room temperature. In between stainings and after final staining cells were washed 3 times with wash buffer. Stained cells were analyzed using a fluorescence microscope.

### Results

Both the mouse monoclonal antibody and the chicken polyclonal antibody serum directed against IBV-Mass could visualize Spike antigen expression in Vero cells. Modifying the C-terminal domain or ER-retention signal seems to change the staining pattern more towards the plasma membrane. Differences in expression levels could not be assessed properly from the other Spike variant antigens using this analysis technique.

IBV Spike Antigen from Plasmid DNA in HELA Cells

To determine if inactivation of the furin cleavage site (ΔFCS), removal of C-terminal domain (ΔCTD) or replacement of the spike protein TMD and CTD by the surface glycoprotein TMD and CTD of VSV, the proline mutation (2P), the addition of a trimerization domain (3M), or the mutation of the ER-retention signal (Y¹¹⁴⁴A) or combinations thereof has any effect on the expression levels of the IBV Spike antigens, HeLa cells were transfected with the pCAGGS expression plasmids that drive the production of the IBV Spike antigens and are used for immunofluorescence assay (IFA).

### Materials & Methods

HELA cells were seeded in DMEM/10% FCS/PS at a density of 100,000 cells/cm2 in 24-well clusters. The following day, cells were transfected with 625 ng pCAGGS2 plasmid DNA using polyethyleneimine (Polysciences Inc.) at a DNA:PEI ratio of 1:10. The transfection mixes were prepared in OptiMEM (Lonza), vortexed for 15 sec and then incubated at room temperature for 20 min. Afterwards, 50 μL mix was added per well and medium was replaced after 7 h of incubation with the cells. At 24 h post transfection 50 μL of culture medium containing DAPI (final dilution per well 1:4000) was added in each well and incubated for 15-30 minutes, after which medium was removed, monolayers were washed one time with DPBS (1× DPBS without Calcium and Magnesium, Lonza) followed by fixing with 3% PFA. After fixing for 1 h cells were washed again with DPBS, permeabilized (or not) for 15 min at 4° C. with 0.5% saponin, and blocked for 1 h in 3% BSA (blocking solution). Afterwards the glass slides were incubated for 1 hour at RT with anti-IBV S mAb (INT-M41-01-03, MSD Animal Health), diluted 1:100 in blocking buffer. Afterwards 3 washing steps of 5 min were performed with 0.05% Tween 20 solution and the secondary antibody (Donkey anti-mouse IgG Alexa488, Molecular probes) was added at a 1:400 dilution in blocking buffer. After another 1 h incubation the cells were washed again 3 times with 0.05% Tween 20 solution and one time with DPBS. Slides were mounted using 10 μL FluorProtect™ reagent (Millipore) and stored at room temperature overnight, before images were collected with the Olympus BX60 fluorescence microscope. All solutions were prepared in DPBS, unless stated otherwise.

### Results

Deleting the C-terminal domain or replacing the IBV TM-CTD for its counterpart of VSV enhances cell surface expression strongly. Moreover, a single amino-acid substitution in the ER-retention signal seems to result in the same increase in cell-surface localization. Introducing the 2P substitutions or mutating the furin cleavage site enhances overall antigen expression levels, while stabilizing the IBV Spike trimer by introducing an additional trimerization domain (3M) reduces Spike expression levels. Notably, both the modification to the furin cleavage site and to the 2P affected the expression levels, whereas the Y¹¹⁴⁴A, CTD, and VSV modifications affected the protein localization (see, Table 1 below). This suggested that combinations of these modifications would be beneficial.

TABLE 1
IN VITRO STUDIES USING AN
IMMUNOFLUORESCENCE ASSAY

| | | Expression |
|---|---|---|
| | Localization | level |
| IBV Spike_wt | Intracellular | + |
| IBV Spike-ΔFCS | Intracellular | ++ |
| IBV Spike-ΔFCS-Y1144A | Cell-surface | ++ |
| IBV Spike-ΔFCS-ΔCTD | Cell-surface | ++ |
| IBV Spike-ΔFCS-VSV | Cell-surface | ++ |
| IBV Spike-ΔFCS-3M | Intracellular | − |
| IBV Spike-ΔFCS-ΔCTD-2P | Cell-surface | +++ |
| mock | — | − |

IBV Spike Antigen from PVAX Plasmid DNA Vaccines in Vero Cells

To determine if combinations of the inactivation of the furin cleavage site (ΔFCS), the modification of CTD, [by either deleting the CTD of the IBV Spike protein, (ΔCTD) or the replacement of the TMD and the CTD of the Spike protein by the TMD and CTD of the surface glycoprotein of VSV], the proline mutation (2P), or the mutation of the ER-retention signal (Y¹¹⁴⁴A) has any effect on the expression levels and/or cell surface localization of the IBV Spike antigens, Vero cells were transfected with the pVAX plasmid DNA vaccines that drive the production of the IBV Spike antigens and used for immunofluorescence assay (IFA).

### Materials & Methods

Vero cells were cultured in DMEM supplemented with 10% FCS, L-Glutamine, and 1% non-essential amino-acids. Cells for transfected were seeded at a density of 25.000 cells/cm² in 24-well clusters in 0.5 ml culture medium and incubated at 37° C., 5% CO₂. Next day, semi-confluent monolayers of Vero cells were transfected with 500 ng pVAX plasmid DNA using Lipofectamine3000 (ThermoFisher) in 50 μl transfection mix according to manufacturer's instructions per well. Twenty-four hours post transfection/infection, cells were washed once with ˜1 ml phosphate-buffered saline (PBS) per well and fixed using either 0.5 ml 96% ethanol per well for 30 minutes at −20° C. or 0.5 ml 4% PFA in phosphate-buffered saline (PBS) for 15 minutes at room temperature. The latter type of fixation assures the cell-membranes are still intact, so that any signal observed must be cell-surface expressed. Cells were washed three times using ˜1 ml wash buffer (PBS+0.15% polysorbate 20) per well and Spike antigens were visualized using either the INT-M41-01-03 mouse monoclonal antibody or a chicken polyclonal antibody serum from Charles River in 0.25 ml IBEIA buffer (PBS+0.05% polysorbate 20+0.1% BSA) for 1 hour at room temperature. Bound antibodies were stained using secondary Goat anti-mouse IgG Alexa488 or Goat anti-chicken IgG Alexa568 antibodies (ThermoFisher) in 0.25 ml IBEIA buffer for 1 hour at room temperature. In between stainings and after final staining cells were washed 3 times with wash buffer. Stained cells were analyzed using a fluorescence microscope.

### Results

Deleting the C-terminal domain (ΔCTD) or the single amino-acid substitution in the ER-retention signal (Y¹¹⁴⁴A) in combination with the 2P substitutions (2P) and furin cleavage site mutation (ΔFCS) seems to result in the most optimal expression levels as well as cell-surface expression. The combination of the TM-CTD substitution for that of VSV in combination with the ΔFCS-2P changes increases expression levels as well as the localization of the protein, but to a lesser extent than the other two combinations. This initial in vitro data show that the combinations of the ΔFCS-2P modifications along with the elimination of the C-terminal domain, the amino-acid substitution in the ER-retention signal (Y¹¹⁴⁴A), or the replacement of the IBV CTD by the CTD of VSV, all resulted in an increase in both cell surface expression, as well as total expression of the IBV spike protein. Interestingly however, the superiority of the replacement of the IBV CTD by the CTD of VSV to the other two modified IBV spike proteins found in the corresponding in vivo data (see below), was not observed in this in vitro data.

TABLE 2
FURTHER IN VITRO STUDIES USING
AN IMMUNOFLUORESCENCE ASSAY

| | CELL-SURFACE | TOTAL |
|---|---|---|
| | EXPRESSION | EXPRESSION |
| | LEVEL | LEVEL |
| IBV Spike_wt | − | + |
| IBV Spike-ΔFCS-2P-ΔCTD | +++ | +++ |
| IBV Spike-ΔFCS-2P-Y1144A | +++ | +++ |
| IBV Spike-ΔFCS-2P-VSV | ++ | ++ |
| mock | − | − |

### Example 4

### Flow-Cytometry Analysis of Expression of IBV Spike Antigens in HEK293 Cells

To determine if modifications to the furin cleavage site (ΔFCS), modification of C-terminal domain (ΔCTD or VSV), the proline mutation (2P), the addition of a trimerization domain (3M), or the mutation of the ER-retention signal (Y¹¹⁴⁴A) has any effect on the expression levels of the IBV Spike antigens, HEK293 cells were transfected with the pCAGGS expression plasmids that drive the production of the IBV Spike antigens as analyzed by flow-cytometry.

### Materials & Methods

HEK293T cells were cultured in DMEM/10% FCS/PS and seeded at a density of 100.000 cells/cm2 in 6-well clusters. The following day, cells were transfected with 2.5 pg pCAGGS2 plasmid DNA using polyethyleneimine (Polysciences Inc.) at a DNA:PEI ratio of 1:10. The transfection mixes were prepared in OptiMEM™ (Lonza), vortexed for 15 sec and then incubated at room temperature for 20 min. Afterwards, 200 μL mix was added per well and medium was replaced after 7 h of incubation with the cells. At 24 h post transfection monolayers were washed one time with DPBS (1× DPBS without Calcium and Magnesium, Lonza) and cells were dissociated adding 0.32 mL TrypLE™ (trypsin replacement reagent, Gibco) for 3-5 min at room temperature. Next cells were mixed with DMEM (up to 1 mL) and 10 μL suspension was used for counting (Invitrogen, Countess II), while the rest was pelleted by centrifugation for 5 min/1000 rpm. Medium was removed and cells were fixed 2% PFA for 20 min, on ice. After fixing cells were pelleted (5 min/2500 rpm/4° C.), permeabilized (or not) for 20 min on ice with 0.5% saponin, and blocked for 1 h in 3% BSA (blocking solution) on ice. Approx. 4×10E5 cells were further used for analysis, from each sample, in duplicate. Blocked cells were moved to round bottom 96-well clusters, pelleted and incubate with the primary antibody (mAbs INT-m41-01-03 or INT-m41-01-08, MSD Animal Health), diluted 1:200 in blocking buffer. Afterwards 3 washing steps of 5 min were performed with 0.05% Tween solution and the secondary antibody (Goat anti-mouse or Donkey anti-mouse IgG Alexa488, Molecular probes) was added at a 1:200 dilution in blocking buffer. After 1 h incubation the cells were washed again 3 times with 0.05% Tween 20 solution and resuspended in FACS buffer (2% BSA, 5 mM EDTA, 0.02% NaN3), before analysis with the CytoFLEX LX™(Beckman Coulter).

### Results

The FACS analysis corroborates the IFA results: the mutation of the ER-retention signal (Y¹¹⁴⁴A), the deletion of the C-terminal domain (ΔCTD) and the presence of the VSV TMD-CTD, in addition to the furin cleavage site mutation (ΔFCS) improves surface expression of the IBV S variants. The highest surface- and total expression was obtained with the variant containing the ΔFCS-2P, as well as the VSV™-CT domain. The 3M variant had the lowest surface expression.

TABLE 3
IN VITRO STUDIES USING FLOW CYTOMETRY ANALYSIS

| | CELL-SURFACE | TOTAL |
|---|---|---|
| | EXPRESSION | EXPRESSION |
| | LEVEL | LEVEL |
| IBV Spike_wt | + | + |
| IBV Spike-ΔFCS | + | + |
| IBV Spike-ΔFCS-Y1144A | ++ | ++ |
| IBV Spike-ΔFCS-ΔCTD | ++ | ++ |
| IBV Spike-ΔFCS-VSV | +++ | +++ |
| IBV Spike-ΔFCS-3M | − | + |
| IBV Spike-ΔFCS-ΔCTD-2P | +++ | +++ |
| IBV Spike-ΔFCS-2P-Y1144A | +++ | +++ |
| IBV Spike-ΔFCS-2P-VSV | +++ | +++ |
| mock | − | − |

### Example 5

### Expression of SARS-CoV-2 Spike Antigens in Cultured Cells Using IFA

To investigate the expression of SARS-CoV-2 Spike antigens in host cells, a series of experiments were performed using different forms for the delivery of the polypeptide according to the invention to host cells. Different staining techniques were applied to visualize the type and the location of those expressions.

SARS-CoV-2 Spike Antigen Using PVAX Plasmid DNA and VEEV RP Vaccines in VERO Cells

To determine if combinations of inactivating the furin cleavage site (ΔFCS), modification of C-terminal domain (ΔCTD or VSV), or the proline mutation (2P) had any effect on the expression levels and/or cell surface localization of the SARS-CoV-2 Spike antigens, Vero cells were transfected with the pVAX plasmid DNA vaccines or infected with the VEEV RPs that drive the production of the Spike antigens and used for immunofluorescence assay (IFA).

### Materials & Methods

Vero cells were cultured in DMEM supplemented with 10% FCS, L-Glutamine, and 1% non-essential amino-acids. Cells for transfected were seeded at a density of 25.000 cells/cm² in 24-well clusters in 0.5 ml culture medium and incubated at 37° C., 5% CO₂. Next day, semi-confluent monolayers of Vero cells were transfected with 500 ng pVAX plasmid DNA using Lipofectamine3000 (ThermoFisher) in 50 μl transfection mix according to manufacturer's instructions or infected with 5.0×10E5 VEEV RPs per well. Twenty-four our post transfection/infection, cells were washed once with −1 ml phosphate-buffered saline (PBS) per well and fixed using 0.5 ml 96% ethanol per well for 30 minutes at −20° C. Cells were washed three times using ˜1 ml wash buffer (PBS+0.15% polysorbate 20) per well and Spike antigens were visualized using either a CR3022 human monoclonal antibody or a rabbit polyclonal antibody directed against the S1A domain of SARS-CoV-2 in 0.25 ml IBEIA buffer (PBS+0.05% polysorbate 20+0.1% BSA) for 1 hour at room temperature. Bound antibodies were stained using secondary Goat anti-human IgG Alexa488 and Goat anti-rabbit IgG Alexa568 antibodies (ThermoFisher) in 0.25 ml IBEIA buffer for 1 hour at room temperature. In between stainings and after final staining cells were washed 3 times with wash buffer. Stained cells were analyzed using a fluorescence microscope.

### Results

Both the CR3022 human monoclonal antibody and the rabbit polyclonal antibody directed against the S1A domain of SARS-CoV-2 could visualize Spike antigen expression in Vero cells. Inactivating the furin cleavage site (ΔFCS) increases antigen expression levels when antigen is produced from the pVAX plasmid DNA vaccine platform as well as the VEEV-RP vaccine platform. No clear differences could be observed in expression levels and/or localization from the other Spike variant antigens using this analysis technique.

TABLE 4
IN VITRO STUDIES USING AN
IMMUNOFLUORESCENCE ASSAY

| | pVAX DNA | VEEV RP |
|---|---|---|
| | vaccine | vaccine |
| SARS-CoV-2 Spike wt | + | ++ |
| SARS-CoV-2 Spike-ΔFCS | ++ | +++ |
| SARS-CoV-2 Spike-ΔFCS-2P | ++ | +++ |
| SARS-CoV-2 Spike-ΔFCS-ΔCTD | ++ | +++ |
| SARS-CoV-2 Spike-ΔFCS-VSV | ++ | +++ |
| SARS-CoV-2 Spike-ΔFCS-VSV-2P | ++ | +++ |
| mock | − | |

SARS-COV-2 Spike Antigen Using PVAX Plasmid DNA and VEEV RP Vaccines in HELA Cells

To determine if combinations of the inactivated furin cleavage site (ΔFCS), modification of C-terminal domain (ΔCTD or VSV), or the proline mutation (2P) had any effect on the expression levels and/or cell surface localization of the SARS-CoV-2 Spike antigens, HELA cells were transfected with the pCAGGS expression plasmids that drive the production of the Spike antigens and used for immunofluorescence assay (IFA).

### Materials & Methods

HeLa cells were seeded in DMEM/10% FCS/PS at a density of 40.000 cells/cm² in 24-well clusters containing glass slides (1 cm diameter). The following day, cells were transfected with 625 ng pCAGGS2 plasmid DNA using polyethyleneimine (Polysciences Inc.) at a DNA:PEI ratio of 1:10. The transfection mixes were prepared in OptiMEM (Lonza), vortexed for 15 sec and then incubated at room temperature for 20 min. Afterwards, 50 μL mix was added per well and medium was replaced after 7 h of incubation with the cells. At 24 h post transfection 50 μL of culture medium containing DAPI (final dilution per well 1:4000) was added in each well and incubated for 15-30 minutes, after which medium was removed, monolayers were washed one time with DPBS (1× DPBS without Calcium and Magnesium, Lonza) followed by fixing with 3% PFA. After fixing for 1 h cells were washed again with DPBS and blocked for 1 h in 3% BSA (blocking solution). Afterwards the glass slides were incubated for 1 hour at RT with anti-SARS CoV2 S human mAb (targeting the RBD), diluted to 10 pg/mL in blocking buffer. Afterwards 3 washing steps of 5 min were performed with 0.05% Tween 20 solution and the secondary antibody (Goat anti-human IgG, Alexa488, Molecular probes) was added at a 1:400 dilution in blocking buffer. After another 1 h incubation the cells were washed again 3 times with 0.05% Tween 20 solution and one time with DPBS. Slides were mounted using 10 μL FluorProtect reagent (Millipore) and stored at room temperature overnight, before images were collected with the Olympus BX60 fluorescence microscope. All solutions were prepared in DPBS, unless stated otherwise.

### Results

Inactivating the furin cleavage site (ΔFCS) increases antigen expression levels marginally when antigen is produced from the pCAGGS expression plasmid. Also, the 2P substitutions, with or without the combination with the TM-CTD replacement of that of VSV, which increases expression levels marginally. The CTD deletion (ΔCTD) of the TM-CTD replacement of that of VSV on its own (VSV) does not have much effect on expression levels.

TABLE 5
SARS-COV-2 SPIKE PROTEIN MODIFICATIONS
EXPRESSION LEVELS

| | Expression |
|---|---|
| | levels |
| SARS-CoV-2 Spike wt | + |
| SARS-CoV-2 Spike-ΔFCS | ++ |
| SARS-CoV-2 Spike-ΔFCS-2P | ++ |
| SARS-CoV-2 Spike-ΔFCS-ΔCTD | + |
| SARS-CoV-2 Spike-ΔFCS-VSV | + |
| SARS-CoV-2 Spike-ΔFCS-VSV-2P | +++ |
| mock | − |

### Example 6

### Flow-Cytometry Analysis of Expression of SARS-CoV Spike Antigens in HEK293 Cells

To determine if combinations of the inactivated furin cleavage site (ΔFCS), modification of C-terminal domain (ΔCTD or VSV), and the proline mutation (2P), have any effect on the expression levels and localization of the Spike antigens, HEK293 cells were transfected with the pCAGGS expression plasmids that drive the production of the SARS-CoV-2 Spike antigens and used for flow-cytometry analysis.

### Materials & Methods

HEK293T cells were cultured in DMEM/10% FCS/PS and seeded at a density of 1×10E5 cells/cm² in 6-well clusters. The following day, cells were transfected with 2.5 pg pCAGGS2 plasmid DNA using polyethyleneimine (Polysciences Inc.) at a DNA:PEI ratio of 1:10. The transfection mixes were prepared in OptiMEM (Lonza), vortexed for 15 sec and then incubated at room temperature for 20 min. Afterwards, 200 μL mix was added per well and medium was replaced after 7 h of incubation with the cells. At 24 h post transfection monolayers were washed one time with DPBS (1× DPBS without Calcium and Magnesium, Lonza) and cells were dissociated by adding 0.32 mL TrypLE (trypsin replacement reagent, Gibco) for 3-5 min at room temperature. Next, cells were mixed by pipetting with DMEM (up to 1 mL) and 10 μL suspension was used for counting (Invitrogen, Countess II), while the rest was pelleted by centrifugation for 5 min/1000 rpm. Medium was removed and cells were fixed with 3% PFA for 20 min, on ice. After fixing cells were pelleted (5 min/2500 rpm/4° C.), permeabilized (or not) for 20 min on ice with 0.5% saponin and blocked for 1 hour in 3% BSA (blocking solution) on ice. Approx. 4×10E5 cells were further used for analysis, from each sample, in duplicate. Blocked cells were moved to round bottom 96-well clusters, pelleted and incubate with the primary antibody (human MAbs 47D11 or CR3022), diluted to 10 pg/mL in blocking buffer. Afterwards 3 washing steps of 5 min were performed with 0.05% Tween 20 solution and the secondary antibody (Goat anti-human IgG, Alexa488, Molecular probes) was added at a 1:400 dilution in blocking buffer. After 1 h incubation the cells were washed again 3 times with 0.05% Tween 20 solution and resuspended in FACS buffer (2% BSA, 5 mM EDTA, 0.02% NaN3), before analysis with the CytoFLEX LX (Beckman Coulter). The results were analyzed with FlowJo v.9 software. All solutions were prepared in DPBS, unless stated otherwise.

### Results

Results tend to vary depending on the hMAb used for detection and the accessibility of specific RBD epitopes. When using the hMAb 47D11 for detection, variants with AFCS have an improved expression especially if the VSV TMD is present. The variant with AFCS and ΔCTD has a lower expression. The data obtained with this assay are corroborated by the immunofluorescence analysis in HeLa cells.

TABLE 6
IN VITRO STUDIES USING FLOW CYTOMETRY ANALYSIS

| | Total | Cell surface |
|---|---|---|
| | expression | expression |
| | levels | levels |
| SARS-CoV-2 Spike_wt | + | + |
| SARS-CoV-2 Spike-ΔFCS | + | + |
| SARS-CoV-2 Spike-ΔFCS-2P | ++ | ++ |
| SARS-CoV-2 Spike-ΔFCS-ΔCTD | + | + |
| SARS-CoV-2 Spike-ΔFCS-VSV | + | ++ |
| SARS-CoV-2 Spike-ΔFCS-VSV-2P | ++ | +++ |
| mock | − | − |

### Example 7

### Immunogenicity of IBV Spike Antigens in Chickens

The in vitro studies described above using modified IBV spike proteins were extended to in vivo studies in chickens. As above, the modified IBV Ma5 spike antigens were designed to be more efficiently expressed on the cell-surface of infected cells. The protective efficacy of viral vectors encoding modified IBV Ma5 antigens, exemplified with the VEEV RNA RP vaccine platform, were evaluated against an IBV M41 challenge. The efficacy of the vaccines was determined by a challenge at 3 weeks post-vaccination and then evaluated based on the degree of ciliary activity of tracheal explants and serology data.

### Materials & Methods

Sixty-six (n=66) birds of 1-day-of-age were assigned to 7 groups (Groups 1-7) according to Table 7 below. At day 1, chickens from Groups 2-8 were either vaccinated with a commercial vaccine by ocular nasal (OCN) administration and with the experimental vaccines listed in Table 7, by intramuscular (IM) administration. At day 22, blood was taken from the chickens in Groups 2, 4, 5, 6, and 7 to determine IBV serology. At day 23, the chickens were subjected to an IBV M41 challenge by an ocular (OC) inoculation. At days 28, 29, and 30, the chickens were euthanized, and their tracheas were used in a ciliostasis assay to determine the vaccine efficacy.

TABLE 7
VACCINATION STUDY WITH MODIFIED IBV Ma5 SPIKE ANTIGENS

| | | | | Vaccine | | | Challenge |
|---|---|---|---|---|---|---|---|
| | | # | | Admin. | Volume | Challenge virus | Admin |
| Group | Isolator | animals | Vaccine | route | ml | (Low dose) | route |
| 1 | A24 | 6 | — | — | — | — | — |
| 2 | A25 | 10 | Nobilis IB Ma5 | OCN | 0.1 | — | — |
| 3 | A26 | 10 | Nobilis IB Ma5 | OCN | 0.1 | IBV-M41 (AG-1559) | OC |
| 4 | A27 | 10 | VEEV-IBV-S wt | IM | 0.25 | IBV-M41 (AG-1559) | OC |
| 5 | A32 | 10 | VEEV-IBV-S-2P-CTD | IM | 0.25 | IBV-M41 (AG-1559) | OC |
| 6 | B36 | 10 | VEEV-IBV-S-2P-Y1144A | IM | 0.25 | IBV-M41 (AG-1559) | OC |
| 7 | C30 | 10 | VEEV-IBV-S-2P-VSV | IM | 0.25 | IBV-M41 (AG-1559) | OC |

All material for vaccination was prepared immediately before the scheduled vaccination. Vaccines were prepared at ambient temperature and administered within 2 hours of preparation. At day 1, the chickens of Groups 3-7 were vaccinated by the ocular nasal route with 0.1 ml vaccine divided over the right eye and the right nostril opening or the IM route with 0.25 ml vaccine in the leg.

Once vaccinated, all groups of the chickens were monitored daily from the day of vaccination to the end of the study for the occurrence of clinical signs of disease or mortality. Chickens showing pain and discomfort that were considered non-transient in nature or likely to become more severe, were euthanized for animal welfare reasons.

At day 22, blood samples (˜2 ml) were taken from the wing vein from all of the chickens of Groups 2, 4, 5, 6, and 7. Blood samples were transported at ambient temperature for evaluation. After clotting at room temperature, the serum was collected by centrifugation of the blood samples at 3000xg for 10 minutes. The serum samples were divided over two sets, subsequently heat inactivated for 30 minutes at 56° C. and then stored at −20° C. until further use. Blood samples collected at day 20 were subjected to a serology assay to determine antibody titers against IBV Ma5 using a commercial ID Screen® Infectious Bronchitis Indirect (IDVet) test.

At day 23, 3 weeks after vaccination, the IBV M41 challenge virus was diluted in Nobilis® Oculo nasal diluent immediately before the scheduled challenge. Subsequently, separate aliquots were prepared for each isolator in which chickens that need to be challenged are housed. The challenge materials were transported on ice in a biosafety transportation box. At day 23, all birds in Groups 3-7 were challenged with challenge strain by the ocular route (4.5 Log 10, 0.1 ml/chicken). The material was equally divided over both eyes. After the challenge, the remains of the challenge virus were analyzed by back titration.

Scheduled post-mortem examinations were performed for trachea isolation after euthanasia of the chickens. Chickens at 4-weeks of age were euthanized by cervical dislocation with prior intramuscular injection of Zoletil™. Shortly after euthanasia of the chickens, the sampling of all tracheas of the chickens in one group were performed with one set of aseptic instruments. The tracheas were excised and individually collected in tubes with pre-warmed (37° C.) medium and kept in an insulated box until transport for the ciliostasis test. The collected tracheas were processed and examined for cilia motility. Tracheas were processed as they come to hand. Ten rings were cut from each trachea i.e., 3 from the top (just below the epiglottis), 4 from the middle, and 3 from the bottom. Once cut, the rings were washed in serum free medium to remove any mucous and placed in a 24-well plate for reading. Rings were read using low-power microscopy. Tracheal rings were scored as not affected (designated as “+”) when at least 50% of the tracheal ring showed vigorous ciliary movement. Tracheal rings with ciliary activity below 50% were scored as “affected” and are designated as “−”. A chicken was considered protected if 90% or more of the rings were not affected.

### Results

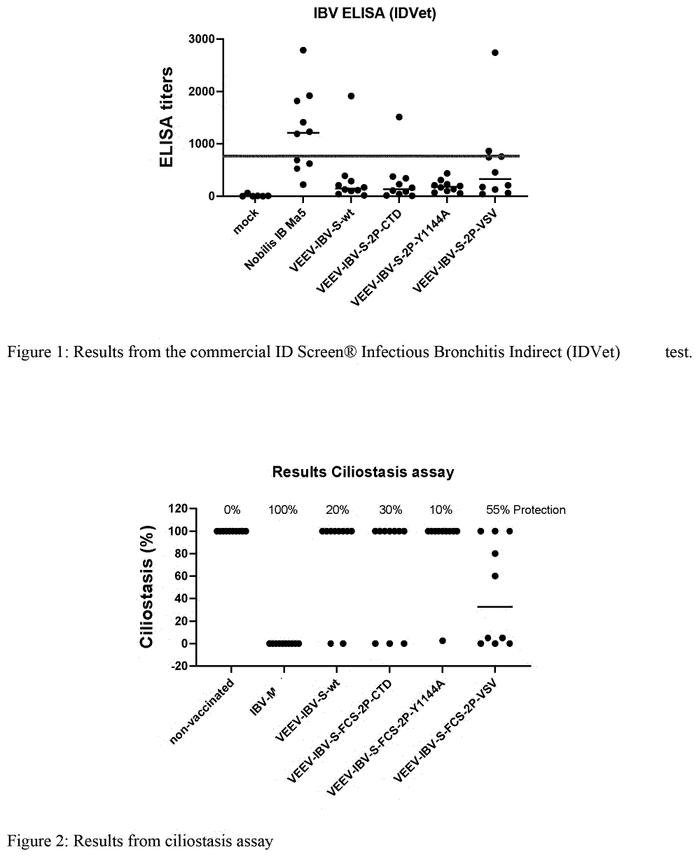

Chickens vaccinated with the Nobilis® IB Ma5 vaccine showed robust seroconversion with 6 out of animals over the threshold of 889 and the group had a mean ELISA titer of 1242. Only one chicken vaccinated with the VEEV RPs expressing the wt IBV Ma5 Spike antigen showed seroconversion and the group had a mean ELISA titer of 336. The combination of ΔFCS+CTD+2P adaptations or the ΔFCS+Y¹¹⁴⁴A+2P adaptations had no effect on the immunogenicity of the IBV Spike antigen. In striking contrast, the ΔFCS+2P+VSV adaptations resulted in more immunogenic antigen in which 4 out of 10 animals showed clear seroconversion and the group had a mean ELISA titer of 617 (see, FIG. 1).

The mean ELISA titers correlated well with the vaccine efficacy where the Nobilis IB Ma5 vaccine resulted in 100% protection, the VEEV RP vaccine expressing the wt IBV Spike antigen only 20% protection, whereas the ΔFCS+2P+VSV adaptations resulted in 55% protection (see FIG. 2).

### Example 8

### Immunogenicity of SARS-CoV-2 Spike Antigens Using VEEV RP Vaccines in Guinea Pigs

To test if the modified SARS-CoV-2 Spike variants gave improved immunogenicity in vivo, experiments was performed in guinea pigs vaccinated with VEEV RP vaccines encoding the different SARS-CoV-2 Spike antigens. The objective of this study was to evaluate the serological efficacy of VEEV RPs encoding the Spike glycoprotein of SARS-CoV-2 in guinea pigs.

### Materials & Methods

For this study, n=35 guinea pigs were used for vaccination at study day (SD) 0, 21, and 42. Vaccines was given intramuscular at a dose of 1.0E7 pfu in 0.3 ml. One group of animals was vaccinated with vaccine mixed with XSOLVE™100 adjuvant. Blood was collected at SD59 and used for serological analysis.

TABLE 8
VACCINATION STUDY WITH MODIFIED SARS-CoV-2 SPIKE ANTIGENS

| | # | | | | Treatment |
|---|---|---|---|---|---|
| Group | Animals | Test Article | Dose | Route | day |
| 1 | 5 | VEEV-SARS-CoV-2-S-wt | 10E7 pfu/0.3 ml | IM | 0, 21, 42 |
| 2 | 5 | VEEV-SARS-CoV-2-S-wt + XSolve ™ | 10E7 pfu/0.6 ml | IM | 0, 21, 42 |
| 3 | 5 | VEEV-SARS-CoV-2-S-ΔFCS | 10E7 pfu/0.3 ml | IM | 0, 21, 42 |
| 4 | 5 | VEEV-SARS-CoV-2-S-ΔFCS-2P | 10E7 pfu/0.3 ml | IM | 0, 21, 42 |
| 5 | 5 | VEEV-SARS-CoV-2-S-ΔFCS-ΔCTD | 10E7 pfu/0.3 ml | IM | 0, 21, 42 |
| 6 | 5 | VEEV-SARS-CoV-2-S-ΔFCS-VSV | 10E7 pfu/0.3 ml | IM | 0, 21, 42 |
| 7 | 5 | VEEV-SARS-CoV-2-S-ΔFCS-2P-VSV | 10E7 pfu/0.3 ml | IM | 0, 21, 42 |
| 8 | 5 | VEEV-GFP | 10E7 pfu/0.3 ml | IM | 0, 21, 42 |

The frozen alphavirus RNA replicon particles were thawed at room temperature prior to vaccination. All guinea pigs were vaccinated intramuscularly (IM) in the thigh or rump with approximately 0.3 mL of the appropriate vaccine preparation. Alternate sites were used for subsequent vaccinations. Group 2 was vaccinated with XSOLVE™100 adjuvant mixed with RNA-P vaccine prior to injection, with a resulting injection volume of ˜0.6 mL.

At the end of the study the guinea pigs were terminally bled for a target minimum yield of 8 to 10 mL of serum. Animals were anesthetized prior to the blood collection using an AVMA-approved method. Following collection, blood samples were held at room temperature for no more than four hours before separation by centrifugation at 1257×g for 30 minutes at 4° C. All serum samples were stored frozen at −20° C. or colder until testing. All serum samples were assayed for SARS-CoV-2 antibodies using a commercial surrogate pseudo-VN test (GenScript).

### Results

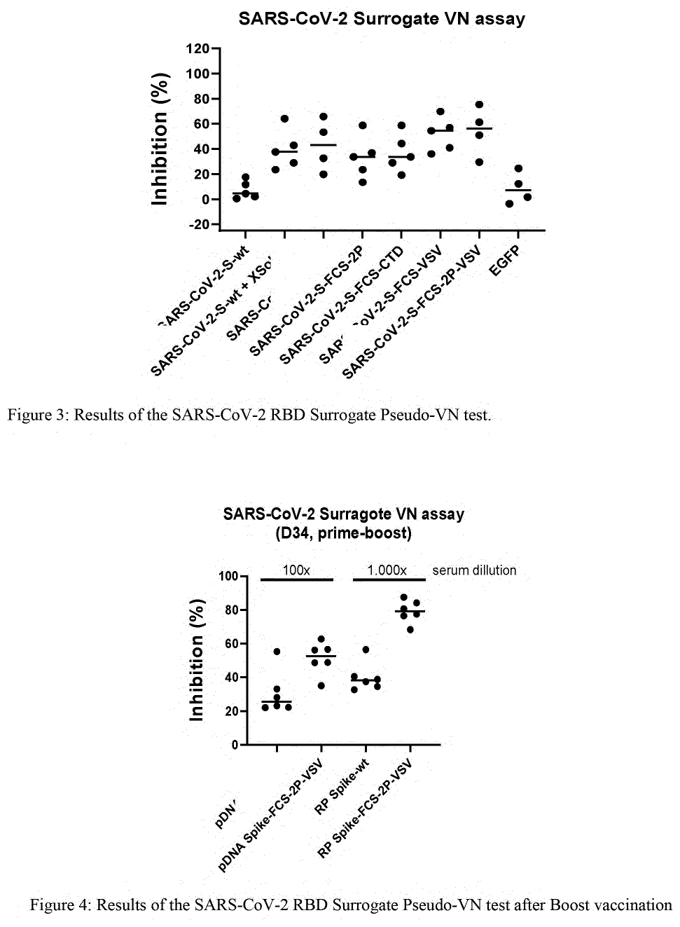

Guinea pigs vaccinated with the VEEV RP expressing the wild type (wt) SARS-CoV-2 Spike antigen only resulted in 7% inhibition, showing very poor seroconversion. Inactivating the furin cleavage site (ΔFCS) of the SARS-CoV-2 Spike antigen resulted in an average of 39% inhibition, while combining the ΔFCS+VSV and ΔFCS+2P+VSV resulted in 52 and 54% inhibition, respectively (see, FIG. 3). Thus, as observed for the IBV Spike antigen, inactivation of the furin cleavage site (ΔFCS) in combination with the VSV modification, with or without the 2P mutation, results in a very immunogenic antigen.

At 3 weeks after prime vaccination, the guinea pigs got a boost vaccination. Results from a surrogate VN-test with blood taken from the guinea pigs is shown in FIG. 4 below. Two points stand out from these data. First, the Spike-2P-VSV variant is much more immunogenic than the Spike-wt antigen. Under the present conditions, the Spike-2P-VSV test shows nearly 100% inhibition. The VEEV RP was surprisingly much more immunogenic than the DNA expression plasmid vaccine.

### Example 9

### Humoral and Cellular Immune Responses Induced by SARS-CoV-2 Spike Antigens Using VEEV RP Vaccines in Guinea Pigs

### Materials and Methods

Animals and Husbandry

Female SPF guinea pigs (Dunkin Hartley) were obtained from Envigo at a minimum weight of 350 grams, randomly allocated to experimental groups, and individually marked using color coded tags. Baseline clinical observations were documented throughout the study period. Baseline clinical observations including body temperatures were documented throughout the study period.

Generation of SARS-CoV-2 Spike Gene RP Vaccines.

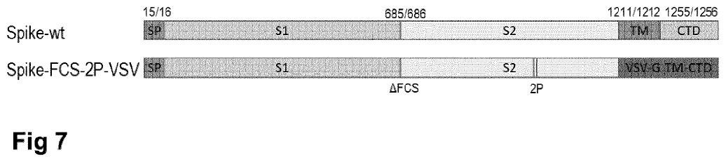

The VEEV replicon vectors used to produce either the SARS-CoV-2 Spike wt or Spike-FCS-2P-VSV gene were constructed as previously described in Example 2 above (see also, FIG. 7). The Spike_wt gene sequence from SARS-CoV-2, strain 2019-nCoV/USA-WI1/2020 (GenBank accession MT039887), and the Spike-FCS-2P-VSV derivative possessing the R⁶⁸²A/R⁶⁸³A (ΔFCS) K⁹⁸⁶P/V⁹⁸⁷P (2P) substitutions and replacement of SARS-CoV-2 spike residues 1212-1273 for residues 463-511 of VSV glycoprotein (GenBank accession YP_009505325, were codon-optimized and synthesized with flanking AscI and PacI sites (ATUM, Newark, CA). The synthetic genes and pVHV vector were each digested with AscI and PacI enzymes and ligated to create vectors “pVHV-SARS-CoV-2-Spike_wt” and “pVHV-SARS-CoV-2-Spike-FCS-2P-VSV” as described in Example 2 above.

Production of TC-83 RNA RPs was conducted by the methods described above (see, Example 2 above). Briefly, pVHV-SARS-CoV-2-Spike_wt and pVHV-SARS-CoV-2-Spike-FCS-2P-VSV replicon vector DNA and helper DNA plasmids were linearized with NotIrestriction enzyme prior to in vitro transcription using RiboMAX™ Express T7 RNA polymerase and cap analog (Promega, Madison, WI). Importantly, the helper RNAs used in the production lack the VEE subgenomic promoter sequence. Purified RNA for the replicon and helper components were combined and mixed with a suspension of Vero cells, electroporated in 4 mm cuvettes, and returned to serum-free culture media. Following overnight incubation, alphavirus RNA replicon particles were purified from the cells and media by passing the suspension through a depth filter, washing with phosphate buffered saline containing 5% sucrose (w/v), and finally eluting the retained RP with 400 mM NaCl+5% sucrose (w/v) buffer or 200 mM Na₂SO₄+5% sucrose (w/v) buffer. Eluted RP were passed through a 0.22 micron membrane filter and dispensed into aliquots for storage prior to assay and lyophilization. A control vaccine was also prepared expressing green fluorescent protein.

The titers of functional RP-spike vaccines were determined by immunofluorescence assay on infected Vero cell monolayers following lyophilization in a stabilizer containing sucrose, NZ Amine and DMEM and storage at 2-8° C. Briefly, the vaccine was serially diluted and added to a Vero cell monolayer culture in 96-well plates and incubated at 37° C. for 18-24 hr. After incubation, the cells were fixed and stained with the primary antibody (anti-VEEV nsp2 monoclonal antibody) followed by a FITC conjugated anti-murine IgG secondary antibody. RNA particles were quantified by counting all positive, fluorescent stained cells in 2 wells per dilution using the Biotek® Cytation™ 5 Imaging Reader.

Guinea Pig Study

SPF guinea pigs with a minimum weight of 350 grams were randomly divided over the non-vaccinated control group, RP-Spike-wt vaccinated group, and RP-Spike-FCS-2P-VSV vaccinated group (n=6 per group). One week after placement, animals remained either non-vaccinated or received a prime vaccination of 1×10E7 RP dose intramuscularly (0.1 ml in each leg muscle). Three weeks after prime vaccination the animals received a booster vaccination of 1×10E7 RP dose intramuscular (0.1 ml in each leg muscle). Six weeks after the booster the vaccination animals received a second booster vaccination and 7 days later animals were sacrificed. Terminal blood was taken for lymphocyte stimulation tests (LST) and trachea were carefully dissected without causing bleedings. Mucus was taken from the inside of the trachea using a swab, taken up in 1 ml of phosphate buffered saline and used to determine mucosal antibody titers. At the day of the booster vaccination, and at 2-week intervals until 6 weeks after boost vaccination, clotted blood was taken using cardiac-puncture and the serum was used to determine systemic antibody titers.

Surrogate Virus Neutralization Assay Guinea Pig Sera

The SARS-CoV-2 Surrogate Virus Neutralization Test Kit from GenScript was used according to the manufacturer's instructions. Briefly, sera were diluted in sample dilution buffer, mixed 1:1 with HRP-RBD, and incubated for 30 minutes at 37° C. Next, samples were put in a 96-well plate containing ACE2 receptor coated on the surface and incubated 15 minutes at 37° C. Unbound HRP-RBD was washed away and remaining horse radish peroxide (HRP) was visualized using 3, 3′, 5, 5′-tetramethylbenzidine (TMB) substrate and measured at OD450.

ELISA for Estimating Anti-RBD and Spike Ectodomain Antibody Titers in Sera

Purified SARS-CoV-2 RBD and Spike ectodomain were diluted in Dulbecco's phosphate-buffered saline (DPBS) [without Ca and Mg, Lonza, 17-512F] and coated onto 96-well plates (MaxiSorp-ThermoFisher, or High binding—Greiner Bio-one) using 10 nM (10 pmols/mL), and incubated overnight at 4° C. The next morning the plates were washed three times with an ELISA plate washer (ImmunoWash 1575, BioRad) using 0.25 mL wash solution/well (DPBS, 0.05% Tween 20), then blocked with 250 μL blocking solution (5% milk—Protifar, Nutricia, 0.1% Tween 20 in DPBS) for 2 hours at RT (room temperature). Afterwards the blocking solution was discarded. Then 4-fold serial dilutions of the sera (prepared in the blocking solution, in duplicates or triplicates) were added to the corresponding wells and incubated for 1 hour at RT. Each plate contained positive control (guinea pig sera diluted to obtain an OD450 of ˜2) and negative control wells. The plates were washed again 3 times before being incubated with the HRP-containing antibody—Goat anti-Guinea pig (IgG-HRPO, Jackson Lab 106-035-003, 1:8000) for 1 hour at RT. The last wash steps were performed, followed by an incubation for 10 minutes at RT with 100 μL/well Super Sensitive TMB (Surmodics, TMBS-1000-01). Reactions were stopped by adding 100 μL/well of 12.5% H₂SO₄ (Millipore, 1.00716.1000). Absorbance at 450 nm was measured at 30 minutes with an ELx808 BioTek plate reader.

T-Cell Stimulation Test (LST)

Blood was collected and lymphocytes were isolated using Sepmate tube (Stemcell) containing Histopaque 1083 according to manufacturer's instructions. Briefly, K3-EDTA blood was diluted 1:2 in RPMI-1640 medium and pelleted for 10 minutes at 1200×g. The cells in the top layer of the tubes were collected, placed in a clean tube containing RPMI-1640 and pelleted for 7 minutes at 400×g. The cells were washed once with RPMI-1640 medium and pelleted for 7 minutes at 400×g. Cell concentrations were counted and 1×10E7 cells were stained with carboxyfluorescein succinimidyl ester (CFSE) for 20 minutes at 37° C. The cells were washed once with RPMI-1640 and 5×10E5 cells from each animal were stimulated with either medium, ConA (10 μg/ml), or purified SARS-CoV-2 S1 antigen (5, 2.5, 1.25, 0.62, 0.31, or 0.15 μg/ml) in duplicate. Three days after stimulation, cell proliferation was measured using the FACS-Verse.

### Results

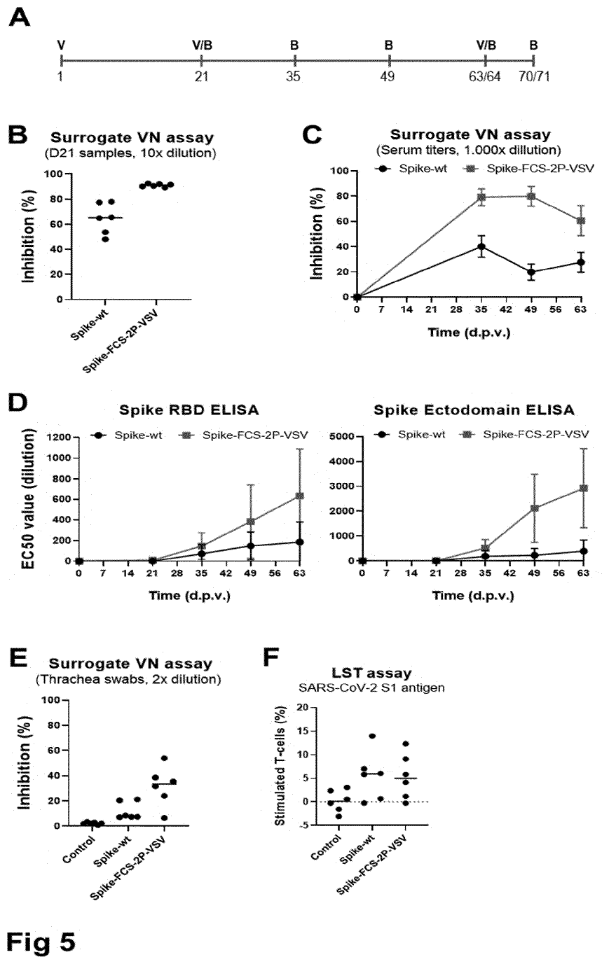

Immunogenicity of the Spike-wt and Spike-FCS-2P-VSV antigens (see, schematic representation in FIG. 7) was assessed in a guinea pig model in which the VEEV RP vector vaccines were administered intramuscularly (FIG. 5A). After prime vaccination, all animals showed seroconversion as assessed by a commercially available surrogate VN test that measures antibody titers interfering with Spike-receptor binding. Clearly higher surrogate VN titers were induced by the Spike-FCS-2P-VSV antigen when compared to the Spike-wt antigen (FIG. 5B). These titers were boosted after the second vaccination with higher titers until the end of the experiment. Consistently, the titers induced by Spike-FCS-2P-VSV antigen were higher in comparison to the RP vaccine producing the Spike-wt antigen (FIGS. 5C-D).

The VEEV RP vector platform is known for its efficient induction of both humoral as well as cellular responses. To assess the level of cellular responses induced by the RP vaccine candidates, a third immunization was performed and seven days later lymphocytes were isolated for a lymphocyte stimulation test (LST). All isolated lymphocytes stimulated with ConA resulted in >80% proliferation titers. In contrast to the differences in humoral responses between the Spike-wt and Spike-FCS-2P-VSV antigens, no differences were observed in levels of SARS-CoV-2 S1 specific T-cell differentiation (FIG. 5E). To determine whether the humoral responses also resulted in mucosal immunity, tracheal swabs were taken at the end of the experiments. Interestingly, surrogate VN titers also were detected in the trachea swabs, and the levels correlated with the systemic antibody levels with superior titers for the Spike-FCS-2P-VSV antigen compared to the Spike-wt antigen (FIG. 5F). These antibody titers suggest that parental vaccination can induce protective mucosal immunity.

### Example 10

### An Alphavirus Replicon-Based Vaccine Expressing a Stabilized Spike Antigen Induces Sterile Immunity and Prevents Transmission of SARS-CoV-2 Between Cats

### Materials and Methods

Animals and Husbandry

Domestic short hair male and female SPF cats were obtained from Marshall BioResources (Waverly, NY), identified by microchip and randomly allocated to experimental groups. Baseline clinical observations including body temperatures were documented throughout the study period.

Generation of SARS-CoV-2 Spike Gene RP Vaccines.

The VEEV replicon vectors used to produce either the SARS-CoV-2 Spike wt or Spike-FCS-2P-VSV gene were constructed as previously described in Example 2 above (see also, Example 9 above, and FIG. 7).

SARS-CoV-2 Challenge Virus and Cell Culture

SARS-CoV-2 strain USA-WA1/2020 (GenBank accession QH060594.1) was isolated from an oropharyngeal swab from a patient with a respiratory illness who had returned from travel to the affected region of China and developed clinical disease (COVID-19) in January 2020 in Washington, USA. The virus was propagated for one passage on Vero cells. To determine the virus titer, serial dilutions of virus were made on Vero cells and plaque forming units quantified by counterstaining with a secondary overlay containing Neutral Red at 24 hours and visualization after 48 hours of incubation.

Placebo Control Vaccine

The placebo vaccine consisted of RNA Particles expressing the green fluorescent protein (gfp or GFP) assayed, lyophilized, and stored at 2-8° C. as described above. Following use, each of the test vaccines were titrated to confirm the vaccination dose.

Feline Serology

Serological responses to SARS-CoV-2 were studied using an in-vitro plaque reduction neutralisation test (PRNT). Briefly serum was inactivated at 56° C. for 30 minutes, serial dilutions of cat serum were prepared and incubated with 100 pfu of SARS-CoV-2 for one hour at 37° C. The virus serum mixtures were then plated onto Vero cells and the number of plaques read by counterstaining with a secondary overlay containing Neutral Red at 24 hours and visualization after 48 hours. Antibody titers were determined as the reciprocal of the highest dilution in which ≥90% of virus was neutralised.

Efficacy Test

Two groups of ten 11-week-old SPF cats were formed and housed separately; one group was vaccinated with 5×10E7 RP-Spike-FCS-2P-VSV administered by the subcutaneous route (0.5 ml per dose) with the other group receiving the same dose of RP-gfp. After three weeks, each group received the same treatment. Twenty-five days following the second vaccination the cats were challenged by using both the intranasal- and oral routes with 3.1×10E5 pfu of SARS-CoV-2 under light sedation. An additional two groups of five SPF cats, which were neither vaccinated nor challenged were used as sentinels by co-housing with each group 1—day post-challenge. All animals were observed for clinical signs indicative of SARS-CoV-2 infection daily for 10 days following the challenge. The clinical signs checked included depression, dyspnea, nasal discharge, ocular discharge, cough, conjunctivitis, and/or sneezing. Body temperatures were recorded on study days 1-11 post-challenge/post-mingling.

Oropharyngeal Swabs

Oropharyngeal swabs for virus isolation were collected from the challenged cats on study days 1 to 7 post-challenge, the swabs were placed in Tris-buffered MEM containing 1% bovine serum albumin containing gentamycin, penicillin, streptomycin and amphotericin B (BA-1 media). Swabs also were collected from the contact sentinels into transport media on study days 2-8 post-challenge to assess the contact spread. The samples were frozen at −50° C. until testing.

Nasal Washes

Nasal wash samples for virus isolation were collected days 1, 2, 3, 5, and 7 post-challenge by instilling 1 ml of BA-1 media into the nares of cats and collecting nasal discharge in a petri dish. Nasal washes were also collected from the contact sentinels on days 2, 3, 4, 6, and 8 post-challenge to assess contact. The samples were frozen at −50° C. until testing.

Blood Samples

Blood samples were taken for sera prior to and 3 weeks post-primary vaccination. In addition, blood samples were taken prior to and 14 days post challenge.

Virus Re-Isolation

All oropharyngeal swabs and nasal washes were tested for virus re-isolation. Confluent monolayers of Vero E6 cells in 6 well plates were washed once with phosphate-buffered saline (PBS) and seeded with 100 μl of serial ten-fold dilutions of swab/wash samples, incubated at 37° C. for one hour then overlaid with 0.5% agarose in MEM containing 2% FBS. A second overlay containing Neutral Red dye was added 24 hours later and plaques were counted at 48 hours. Viral titers were recorded in Log 10 pfu/ml.

Results

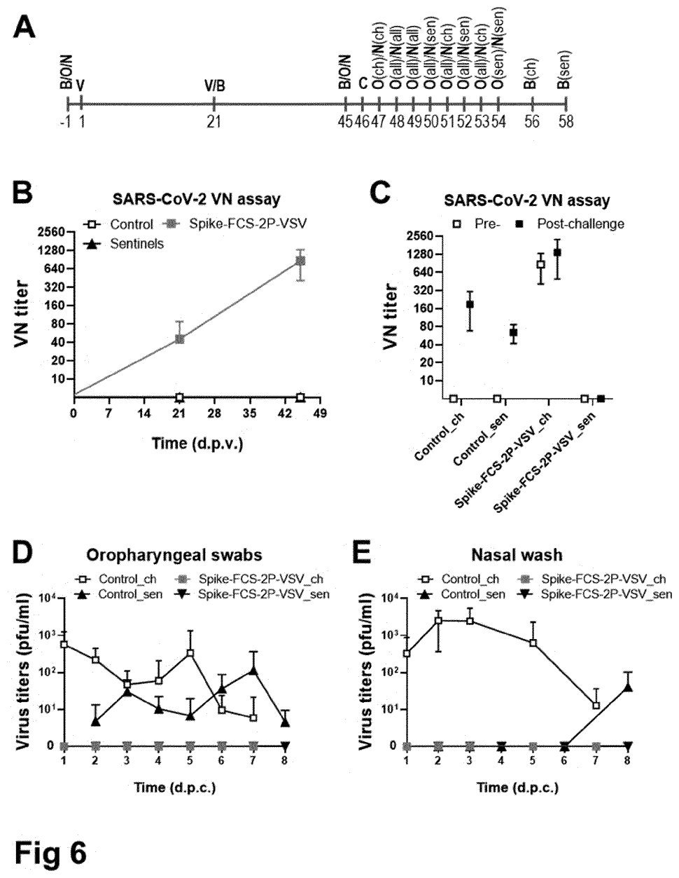

To determine vaccine efficacy, cats were either vaccinated with a RP vaccine producing enhanced green fluorescent protein (EGFP) as a Control, the optimized SARS-CoV-2 Spike antigen (Spike-FCS-2P-VSV) or remained non-vaccinated (sentinels). Three weeks post booster vaccination, cats were exposed to a mucosal SARS-CoV-2 challenge and samples were taken as outlined in FIG. 6A.

Following vaccination, no adverse reactions were detected in any of the cats at any timepoint. The RP vaccine producing the Spike-FCS-2P-VSV antigen was able to induce a virus neutralising antibody titer in all cats after a single vaccination, which was boosted after the second vaccination and maintained levels until the challenge 3.5 weeks later (FIG. 6B). Non-vaccinated sentinel animals remained negative at all times up until challenge. Neither the challenged-nor the sentinel cats demonstrated any clinical signs post challenge. However, nine out of ten of the non-vaccinated challenged cats shed virus orally (FIG. 6D) and nasally (FIG. 6E) one day after challenge, and for at least 3 days during the observation period. These data show that the mucosal SARS-CoV-2 challenge results in efficient virus replication in the respiratory tract. Higher and more consistent virus shed was detected from the nasal washes, whereas the oropharyngeal swabs demonstrated a less consistent pattern. Interestingly, virus shed was also detected from the nasal washes in two of the non-vaccinated sentinels placed with the non-vaccinated controls one day after challenge. Moreover, all five sentinel animals shed virus via the oral route for at least two days, which demonstrates the efficient spread of the virus from non-vaccinated challenged- to sentinel animals (FIG. 6D).

None of the vaccinated cats shed any detectable virus orally (FIG. 6D) or nasally (FIG. 6E) at any timepoint after the challenge. The results indicate that the vaccine prevented infection. Also, no virus was detected in the non-vaccinated sentinels housed with the vaccinated cats as would be expected considering the lack of challenge virus replication in the vaccinated cats. Analysis of virus neutralising antibody titers post challenge confirmed that both non-vaccinated challenged and sentinel animals were efficiently infected (FIG. 6C). In direct contrast, no seroconversion was observed in the sentinel animals housed with the vaccinated cats. Thus, the VEEV RP vaccine producing the Spike-FCS-2P-VSV antigen both (i) induces sterile immunity and (ii) prevents the transmission of virus from infected to naïve cats.

These results have meanwhile been published as: Langereis et al., 2021, npj Vaccines vol. 6, no. 122; https://doi.org/10.1038/s41541-021-00390-9.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, and all molecular weight or molecular mass values, given for nucleic acids or polypeptides are approximate, and are provided for description.

### Example 11

### Mutation of Further Coronaviral Spike Antigens

To investigate the expression of chimeric spike proteins from further Coronaviruses, several more experiments were performed. Among others, the genes encoding the spike proteins of bovine coronavirus (BCoV) and of the swine coronavirus causing SADS were mutated to improve their stability and (surface-)expression.

### Materials & Methods

Experiments to show expression by FACS were done essentially as described above: Vero cells were amplified and plated out. Plasmids containing the (mutated) spike gene to be tested were transfected into the Vero cells using Lipofectamine, and cultured. Next cells were harvested and fixed. Cells were permeabilised with detergent or not, to be able to differentiate between respectively total/internal- or surface expression. The antibodies used were, for BCoV: a mouse monoclonal anti-BCoV Spike, and a Goat-anti-Mouse IgG-A488 conjugated antibody; for SADS-CoV: a polyclonal rabbit anti-SADS-CoV S1 antibody, and a Goat-anti-Rabbit IgG-A488 conjugated antibody.

The BCoV spike protein gene used, see SEQ ID NO: 16 with translation in SEQ ID NO: 17, is a consensus sequence that was assembled from 130 BCoV spike sequences from 2016-2021 available in public databases.

The SADS-CoV spike gene, see SEQ ID NO: 18 with translation in SEQ ID NO: 19, is derived from the porcine enteric alphacoronavirus of strain GDS04, the genome of which is available from GenBank acc. nr.: MF167434.

The spike protein mutations tested for the BCoV (consensus) spike were similar to those as tested for the IBV and SARS-CoV-2 spike proteins described above: FCS, 2P, and VSV-TM/CT. In addition, the BCoV consensus TMD-CTD region was replaced by that from Influenza virus HA protein, strain A/Puerto Rico/8/1934 (H1N1), for which the HA gene sequence is available from GenBank acc.nr.: V01088.

The SADS-CoV spike was mutated by replacement of its TMD-CTD region by that from VSV G protein.

Specific mutations made to the BCoV spike:
the ‘FCS’ mutated BCoV consensus spike gene, has the mutated (inactivated) furin cleavage site, and is presented in SEQ ID NO: 20, which incorporates the mutation at its nucleotides 2290-2295 and 2299-2304.
The ‘FCS-2P’ mutated BCoV consensus spike gene, incorporates next to the inactivated furin cleavage site also the stabilising two prolines. The sequence is presented in SEQ ID NO: 21, which has the 2P mutation at its nucleotides 3238-3243.
The ‘FCS-IAV-TM/CT’ mutated BCoV consensus spike gene, incorporates next to the inactivated furin cleavage site also the replacement of the BCoV consensus TMD-CTD region by that from Influenza virus HA protein, as presented in SEQ ID NO: 22, which has the Influenza HA TM/CT region at its nucleotides 3922-4029.
The ‘FCS-VSV-TM/CT’ mutated BCoV consensus spike gene, incorporates next to the inactivated furin cleavage site also the replacement of the BCoV spike protein consensus TMD-CTD region by that from VSV G protein, see SEQ ID NO: 23, which has the VSV G protein TM/CT region at its nucleotides 3922-4068.
The construct ‘FCS-2P-VSV-TM/CT’ combines above mutations.

Specific Mutations Made to the SADS-CoV Spike:
The ‘VSV-TM/CT’ mutated SADS-CoV spike gene, incorporates the replacement of the SADS-CoV TMD-CTD region by that from VSV G protein, see SEQ ID NO: 24, which has the VSV TM/CT region at its nucleotides 3205-3351.

### Results

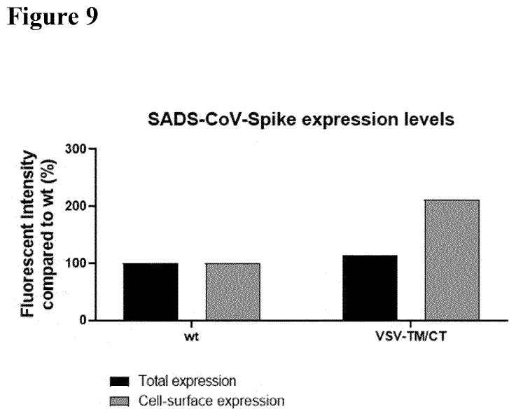

The effects of the different mutations on the expression of spike protein from BCoV and from SADS-CoV, were compared to the expression-level of their respective unmutated spike proteins (‘wt’), which was set at 100%. Results for chimeric spike proteins from BCoV are presented in FIG. 8, and from SADS-CoV in FIG. 9.

As is clear from FIGS. 8 and 9, the results for BCoV and SADS-CoV spike protein follow the results seen for IBV and SARS-CoV2 spike proteins described above. For all spike proteins, the replacement of the TMD-CTD region by that from a surface glycoprotein of a budding virus (e.g. VSV G protein) is beneficial to the total expression level, but is especially advantageous for the level of expression on the surface of the host cells. This was also observed after the use of the TMID-CTD region from Influenza virus HA protein. Other modifications, such as removal of the furin cleavage signal (‘FCS’), and stabilisation of the pre-fusion conformation (‘2P’), have similar effects: some increase of the total spike protein expression level, and strong to very strong increase of spike protein expression on the cell-surface.

These results for BCoV and SADS-CoV spike protein thus confirm and expand upon the advantageous effects of the invention as described herein.

1. A recombinant vector encoding a chimeric coronavirus spike protein that comprises a spike protein originating from a coronavirus, and a transmembrane domain (TMD) and a C-terminal domain (CTD) of a surface glycoprotein originating from a budding virus that buds from a host cell's plasma membrane (BVₚₘ), in place of a TMD and a CTD of the coronavirus spike protein; wherein when the recombinant vector is a recombinant BVₚₘ, the TMD and the CTD of the surface glycoprotein originate from a virus species that is different from that of the recombinant BVₚₘ, and wherein the coronavirus spike protein originates from a coronavirus selected from the group consisting of an infectious bronchitis virus (IBV).

2. (canceled)

3. The recombinant vector of claim 1, wherein the surface glycoprotein originates from a BVₚₘ species that is selected from the group consisting of the glycoprotein (G protein) of a vesicular stomatitis virus (VSV).

4. (canceled)

5. The recombinant vector of claim 1, wherein the chimeric coronavirus spike protein comprises an inactivated furin cleavage site.

6. The recombinant vector claim 1, wherein the chimeric coronavirus spike protein comprises a central helix that is further stabilized in a prefusion state due to two consecutive amino acid residues at the beginning of the central helix being replaced by a pair of proline residues (2P).

7.-14. (canceled)

15. The recombinant vector of claim 1, wherein the IBV is a member of a serotype selected from the group consisting of a Massachusetts serotype, a 4/91serotype, and a QX serotype.

16. The recombinant vector of claim 15, wherein the IBV is an IBV-Ma5 strain.

17. The recombinant vector of claim 15, wherein the chimeric coronavirus spike protein comprises 90% or greater identity with amino acid residues 19 to 1091 of the amino acid sequence of SEQ ID NO: 4, over the same range of amino acid residues; and wherein said chimeric coronavirus spike protein comprises an inactivated furin cleavage site.

18. The recombinant vector of claim 15, wherein the chimeric coronavirus spike protein comprises 90% or greater identity with amino acid residues 19 to 1091 of the amino acid sequence of SEQ ID NO: 6, over the same range of amino acid residues; wherein said chimeric coronavirus spike protein comprises an inactivated furin cleavage site; and wherein the alanine (A) residue at position 859 and the isoleucine (I) residue at position 860 of SEQ ID NO: 6 are replaced by a pair of proline residues (2P).

19. The recombinant vector of claim 17, wherein the chimeric coronavirus spike protein further comprises 90% or greater identity with amino acid residues 1092 to 1140 of the amino acid sequence of SEQ ID NOs: 4 or 6, over the same range of amino acid residues.

20. The recombinant vector of claim 1, that is a recombinant expression vector selected from the group consisting of a recombinant viral vector and a DNA expression plasmid.

21. The recombinant expression vector of claim 20, that is a recombinant viral vector selected from the group consisting of a recombinant herpesvirus of turkeys (HVT), a recombinant attenuated Marek's disease virus 1 (MDV1), a recombinant Marek's disease virus 2 (MDV2), a recombinant MV, a recombinant NDV, and an alphavirus RNA replicon particle (RP).

22. The recombinant viral vector of claim 21, that is a HVT.

23. The recombinant viral vector of claim 21, wherein the alphavirus RNA replicon particle is a Venezuelan Equine Encephalitis virus (VEEV) replicon particle.

24. The recombinant expression vector of claim 20, that is a DNA expression plasmid.

25. The recombinant expression vector of claim 24, that encodes an RNA replicon, wherein the RNA replicon is a VEEV RNA replicon.

26.-27. (canceled)

28. An immunogenic composition comprising the recombinant vector of claim 1, the recombinant viral vector selected from the group consisting of a recombinant herpesvirus of turkeys (HVT), a recombinant attenuated Marek's disease virus 1 (MDV1), a recombinant Marek's disease virus 2 (MDV2), a recombinant MV, a recombinant NDV, and an alphavirus RNA replicon particle (RP), a DNA expression plasmid, or a synthetic mRNA, and a pharmaceutically acceptable carrier.

29.-33. (canceled)

34. A vaccine to aid in the protection of an avian from infectious bronchitis due to an infection of IBV in the avian comprising the recombinant vector of claim 1, the recombinant viral vector selected from the group consisting of a recombinant herpesvirus of turkeys (HVT), a recombinant attenuated Marek's disease virus 1 (MDV1), a recombinant Marek's disease virus 2 (MDV2), a recombinant MV, a recombinant NDV, and an alphavirus RNA replicon particle (RP), a DNA expression plasmid, or a synthetic mRNA, and a pharmaceutically acceptable carrier.

35. The vaccine of claim 34, which further comprises at least one non-IBV antigen for eliciting protective immunity to a non-IBV avian pathogen.

36. The vaccine of claim 34, that comprises an adjuvant.

37. The vaccine of claim 34, that is a non-adjuvanted vaccine.

38.-46. (canceled)

47. A chimeric coronavirus spike protein that comprises a spike protein originating from an IBV, and a TMD and a CTD of a surface glycoprotein originating from a vesicular stomatitis virus, in place of a TMD and a CTD of the IBV spike protein.

48. The chimeric coronavirus spike protein of claim 47, wherein the chimeric coronavirus spike protein comprises 90% or greater identity with amino acid residues 19 to 1091 of the amino acid sequence of SEQ ID NO: 4, over the same range of amino acid residues; and wherein said chimeric coronavirus spike protein comprises an inactivated furin cleavage site.

49. The chimeric coronavirus spike protein of claim 48, wherein the chimeric coronavirus spike protein comprises 90% or greater identity with amino acid residues 19 to 1091 of the amino acid sequence of SEQ ID NO: 6, over the same range of amino acid residues; wherein said chimeric coronavirus spike protein comprises an inactivated furin cleavage site; and wherein the alanine (A) residue at position 859 and the isoleucine (I) residue at position 860 of SEQ ID NO: 6 are replaced by a pair of proline residues (2P).

50. The chimeric coronavirus spike protein of claim 48, wherein the chimeric coronavirus spike protein further comprises 90% or greater identity with amino acid residues 1092 to 1140 of the amino acid sequence of SEQ ID NOs: 4 or 6, over the same range of amino acid residues.

51. A nucleic acid encoding the chimeric coronavirus spike protein claim 47.

52.-57. (canceled)
